(19) United States
(12) Patent Application Publication
(10) Pub. No.: US 2024/0409973 A1
(43) Pub. Date: Dec. 12, 2024

(54) HEPAROSAN-PRODUCING RECOMBINANT CELLS

(71) Applicant: GIVAUDAN SA, VERNIER (CH)

(72) Inventors: Dominique LOUIS, ORSAY (FR); Karine JAILLARDON, ORSAY (FR); Muriel MERKAMM, ORSAY (FR); Dominique THOMAS, Argenteuil (FR)

(21) Appl. No.: 18/553,345

(22) PCT Filed: Mar. 31, 2022
(86) PCT No.: PCT/EP2022/058568

§ 371 Date: Sep. 29, 2023

(30) Foreign Application Priority Data
Apr. 1, 2021 (EP) 21166744.9

(51) Int. Cl. C12P 19/26; C12N 9/04; C12N 9/24; C12N 9/90

(52) U.S. Cl. CPC C12P 19/26; C12N 9/0006; C12N 9/2402; C12N 9/90; C12Y 101/01022; C12Y 501/03017

(57) ABSTRACT

The present invention relates to the field of bio-production of heparosan. There is a need in the art for heparosan production methods allowing its highly efficient synthesis and secretion. The solution proposed in the present invention is the use of a genetically modified cell comprising many modifications as described in the present text. The present invention further proposes methods allowing the bio-production of heparosan having a controlled molecular weight using the genetically modified cells of the invention.

### FIELD OF THE INVENTION

The present invention relates to the field of bio-production of heparosan.

### REFERENCE TO SEQUENCE LISTING

This application includes as the Sequence Listing the complete contents of the accompanying text file “second_sub_sequence_listing”, created Jul. 30, 2024, containing 133,054 bytes, hereby incorporated by reference.

### BACKGROUND OF THE INVENTION

Heparosan, also known as N-acetylheparosan, is the natural eukaryotic biosynthesis precursor of heparin and heparan sulfate found in animals from hydra to vertebrates. It is more particularly a polysaccharide with the repeating diassachride unit [→4) β-D-glucuronic acid (GlcA) (1→4)N-acetyl-a-D-glucosamine (GlcNAc) (1→]ₙ. In nature, the Escherichia coli K5 and the Pasteurella multocida type D strains produce heparosan as a capsular polysaccharide (Lindahl U. et al. (1998) J. Biol. Chem., 273 (39): 24979-24982).

Derivates from heparosan bind to a variety of extremely important polypeptides including hemostasis factors (e.g., antithrombin III, thrombin), growth factors (e.g., EGF, VEGF), and chemokines (e.g., IL-8, platelet factor 4) as well as the adhesive proteins for viral pathogens (e.g., herpes, Dengue fever). Among these derivatives, heparin, one of the most widely used anticoagulant or antithrombotic drug, is for example useful in therapeutic treatments of thromboembolism and disseminated intravascular coagulation (DIC), extracorporeal circulation and prevention of blood coagulation during artificial dialysis. In addition, recent studies suggest that heparin may have significant antineoplastic activity, separate and distinct from its anticoagulant activity, while other studies indicate a role for heparin in treating inflammation, infertility, and infectious disease.

Today's main known sources of heparin are extractions from animal tissue, such as porcine intestinal mucosa, with the attendant risks of adulteration and contamination.

Fermentation methods for preparing heparosan from microorganisms are particularly of interest since they facilitate the production of a large quantity of heparosan through possible scale-up, at a reduced cost. Moreover, contrary to isolating heparosan and its derivatives from animal sources which provide heparosan of very high molecular weights, microbial fermentation allows to control, to a certain extent, the size of the starting molecular weight. This avoids the need for further fractionation steps by mechanical, physical or chemical means.

In this aim, recombinant Bacillus megaterium host cells have shown the ability to produce heparosan in the range of 10 to 300 KDa (A. Williams et al. Microb. Cell. Fact. 2009 Aug. 12; 18 (1): 132). Similarly, Escherichia coli K5 strains shown the ability to produce heparosan (Wang Z. et al. (2010) Biotechnol. Bioeng., 107 (6): 964-973), but there are still many issues that remain to be resolved, such as the improvement of the substrate consumption rate and the increase of the oxygen supply in fermentation tank.

Contrary to other microorganisms that are commonly used for the production of biological molecules, yeasts are generally recognised as safe. They can grow rapidly and be cultivated at higher density, compared to bacteria, and do not require an aseptic environment. Furthermore, yeast cells can be more easily separated from the culture medium than bacteria, greatly simplifying the process for product extraction and purification. Finally, yeasts present the advantage of being more resistant to pH changes in the culture medium, and as such represent stronger fermentation systems.

However, among yeasts, distinguishing features between species may also present certain challenges. This is mainly due to the difference in metabolisms between the species. For example, genome integrations in P. pastoris are stable but often a high variability of clones from one transformation is encoutenred, displaying various productivity characteristics or changes in their physiology. This requires a time-consuming screening process so as to find the clone with the optimal features for the desired application. This clonal variability is an important drawback for the further development of P. pastoris as a platform for producing value-added chemicals. Another drawback of using P. pastoris is that it is a methanotrophic organism.

On the contrary, Saccharomyces cerevisiae is particularly useful as a tool for the production of molecules of interest since it has a long safe history when it comes to being used by humans (such as in wine, beer, or bread) and as such is a well-established model whose genetic information is well known in the art. S. cerevisiae further presents the advantage of being generally recognised as safe for humans and animals. What's more, the acidification of the medium which occurs when cultivating this yeast reduces the possibility of contamination of the bio fermenters, and therefore eliminates the need to add antibiotics to the medium. Finally, many genetic tools have been developed allowing for stable modifications of its genome (integration within the chromosome).

Accordingly, there is still a need in the art for further heparosan production methods allowing its highly efficient synthesis and secretion. In particular, there is still a need to provide production methods that are cost-efficient and supply heparosan which is safe for human application.

In a particular case, there is still a need in the art for heparosan production methods allowing to obtain large amounts of heparosan of a particular and controlled size.

### SUMMARY OF THE INVENTION

The present invention accordingly relates to the following items:

Item 1: A recombinant yeast cell producing heparosan wherein the recombinant cell comprises:
(a) one or more recombinant nucleic acids encoding a polypeptide having a heparosan synthase (HSS) activity; and
(b) one or more recombinant nucleic acids encoding a polypeptide having a UDP-Glucose dehydrogenase (UDP-GlcDH or HASB) activity.

As illustrated in the examples, the recombinant yeasts of the invention allow for the production of heparosan in a yeast cell which is not naturally able to produce heparosan. Said advantageous properties can be further increased by recombining the yeast with additional modifications described here-after.

Item 2: The recombinant cell according to item 1 wherein the recombinant cell comprises a nucleic acid encoding a polypeptide having a glucuronosyl-disulfoglucosamine glucuronidase activity, wherein the polypeptide having glucuronosyl-disulfoglucosamine glucuronidase activity comprises a secretion signal and optionally an anchoring signal.

Item 3: A recombinant host cell producing heparosan wherein the recombinant cell comprises:
(a) one or more recombinant nucleic acids encoding a polypeptide having a heparosan synthase (HSS) activity;
(b) one or more recombinant nucleic acids encoding a polypeptide having a UDP-Glucose dehydrogenase (UDP-GlcDH or HASB) activity; and
(c) one or more recombinant nucleic acids encoding a polypeptide having a glucuronosyl-disulfoglucosamine glucuronidase activity, wherein the polypeptide having a glucuronosyl-disulfoglucosamine glucuronidase activity comprises a secretion signal, and optionally an anchoring signal, so that heparosan, in particular of a desired molecular weight is produced by the host cell.

Item 4: The recombinant cell according to item 2 or 3, wherein the nucleic acid encoding a polypeptide having glucuronosyl-disulfoglucosamine glucuronidase activity is obtained or derived from Pedobacter heparinus.

Item 5: The recombinant cell according to any one of items 2 to 4, wherein the nucleic acid encoding a polypeptide having a glucuronosyl-disulfoglucosamine glucuronidase activity is under the control of a promoter selected from the group consisting of pTEF1, pCCW12, pCCW12.sba, pCCW12.Sar, pPDC1, pTEF3, pTDH3, pNUP57 and pCCW10.ago.

Item 6: The recombinant cell according to any one of items 2 to 5, wherein the molecular weight of the heparosan is in the range of less than 50 kDa, preferably is in the range of about 20 kDa to about 50 kDa.

Item 7: The recombinant cell according to any one of items 2 to 5, wherein the molecular weight of the heparosan is in the range of greater than 50 kDa, preferably in the range of about 50 kDa to about 250 kD.

Item 8: The recombinant cell according to any one of items 2 to 5 wherein the molecular weight of the heparosan is in the range of greater than 100 kDa, preferably is in the range of about 100 kDa to about 1500 kDa.

Item 9: The recombinant cell according to any one of items 1 to 8, wherein the nucleic acid encoding a polypeptide having a UDP-Glucose dehydrogenase (UDP-GlcDH or HASB) activity is obtained or derived from at least one of Arabidopsis thaliana, Chlorella virus PBCV1 or Streptococcus zooepidemicus, and is in particular from Arabidopsis thaliana or Chlorella virus PBCV1.

Item 10: The recombinant cell according to any one of items 1 to 9, wherein the nucleic acid encoding a polypeptide having a heparosan synthase (HSS) activity is obtained or derived from Pasteurella multocida.

Item 11: The recombinant cell according to any one of items 1 to 10, wherein the recombinant cell further comprises at least one recombinant nucleic acid encoding one or more of:
(i) a polypeptide having a glutamine-fructose-6-phosphate amidotransferase (GFA1) activity; and/or
(ii) a polypeptide having a UDP-N-acetylglucosamine pyrophosphorylase (QRI1) activity.

Item 12: The recombinant cell according to any one of items 1 to 11, wherein the recombinant cell further comprises at least one recombinant nucleic acid encoding one or more of:
(i) a polypeptide having a Phosphoglucomutase-1 (PGM1) activity; and/or
(ii) a polypeptide having a UTP-glucose-1-phosphate uridylyltransferase (UGP1) activity; and/or
(iii) a polypeptide having a Glucosamine-6-phosphate N-acetyltransferase (GNA1) activity; and/or
(iv) a polypeptide having a phosphoacetylglucosamine mutase (PCM1) activity.

Item 13: The recombinant cell according to item 11 or 12, wherein:
the nucleic acid encoding the polypeptide having glutamine-fructose-6-phosphate amidotransferase (GFA1) activity; and/or
the nucleic acid encoding the polypeptide having UDP-N-acetylglucosamine pyrophosphorylase (QRI1) activity; and/or
the nucleic acid encoding the polypeptide having Phosphoglucomutase-1 (PGM1) activity; and/or
the nucleic acid encoding the polypeptide having UTP-glucose-1-phosphate uridylyltransferase (UGP1) activity; and/or
the nucleic acid encoding the polypeptide having Glucosamine-6-phosphate N-acetyltransferase (GNA1) activity; and/or
the nucleic acid encoding the polypeptide having phosphoacetylglucosamine mutase (PCM1);
are obtained or derived from Saccharomyces cerevisiae.

Item 14: The recombinant host cell according to any one of items 3 to 13, wherein the recombinant host cell is a yeast.

Item 15: The recombinant cell according to any one of items 1 to 14, wherein the recombinant cell belongs to the Saccharomyces genus, or to the Candida genus, or to the Kluyveromyces genus, or to the Ogataea genus, or to the Yarrowia genus, or to the Debaryomyces genus, or to the Ashbya genus, and in particular belongs to the Saccharomyces genus.

Item 16: The recombinant cell according to item 15, wherein the recombinant cell is selected from the group consisting of Saccharomyces cerevisiae, Saccharomyces boulardii, Saccharomyces bayanus, Saccharomyces paradoxus, Saccharomyces mikatae, Saccharomyces castelli, Candida albicans, Candida glabrata, Candida tropicalis, Kluyveromyces lactis, Kluyveromyces marxianus, Kluyveromyces polysporus, Kluyveromyces thermotolerens, Ogataea polymorpha, Yarrowia lypolytica, Debaryomyces hansenii, and Ashbya gossypii, and is preferably Saccharomyces cerevisiae.

Item 17: A method of producing heparosan comprising:
(a) cultivating a recombinant cell as defined in any one of items 1 to 16 in a cultivation medium for a time sufficient to produce heparosan; and
(b) optionally isolating or recovering the heparosan from the recombinant cell and/or from the cultivation medium.

Item 18: The method according to item 17, wherein the heparosan has a molecular weight of from about 20 kDa to about 50 kDa.

Item 19: The method according to item 17, wherein the heparosan has a molecular weight of from about 50 kDa to about 150 kDa.

Item 20: The method according to item 17, wherein the heparosan has a molecular weight of from about 150 kDa to about 1500 kDa.

Item 21: The method according to any one of items 17 to 20, wherein the recombinant cell is a yeast belonging to the Saccharomyces genus, and in particular is Saccharomyces cerevisiae.

Item 22: The method according to any one of items 17 to 21, wherein the time sufficient to produce heparosan is a period of from about 35 to about 50 hours, preferably from about 40 to about 50 hours, preferably of about 48 hours.

Item 23: The method according to any one of items 17 to 22, wherein the molecular weight of the produced heparosan is controlled by regulating the pH of the cultivation medium during step (a).

Item 24: The method according to any one of items 17 to 23, wherein the method is carried out on an industrial scale, preferably where the cultivation medium is at least about 100 L, more preferably is in the range of about 1000 L to about 3000 L, even more preferably is about 10,000 L or even more preferably is about 100,000 L, or even is about 250,000 L.

Item 25: Heparosan obtainable from a recombinant cell according to any one of items 1 to 15 or from the method according to any one of items 17 to 24.

Item 26: A cultivation medium comprising the heparosan according to item 25.

Item 27: A composition comprising the heparosan according to item 25.

Item 28: An industrial product or a consumer product or a consumable comprising (i) the heparosan according to item 25 having a molecular weight as defined in any one of items 6 to 8, (ii) the cultivation medium of item 26 or (iii) the composition of item 27.

Item 29: The industrial product or the consumer product or the consumable of item 28, wherein the industrial product or the consumer product or the consumable is a cosmetic product, a flavour product, a fragrance product, a food product, a food, a beverage, a texturant, a pharmaceutical composition, a dietary supplement, a nutraceutical, a cleaning product, a dental and/or an oral hygiene composition.

Item 30: Use of a recombinant cell as defined in any one of items 1 to 116 for the production of heparosan having a molecular weight in the range of from about 20 kDa to about 50 kDa or from about 50 kDa to about 1000 kDa.

Certain embodiments of the present invention may provide one or more of the following advantages:
a process for producing heparosan of controlled molecular weight;
a process for producing heparosan of controlled molecular weight in a Saccharomyces yeast cell; and
a process for producing heparosan of controlled molecular weight by varying genetic parameters (eg regulatory sequences) and/or process parameters (pH or fermentation time).

### BRIEF DESCRIPTION OF THE FIGURES

FIG. 1 shows a schematic pathway for the production of heparosan.

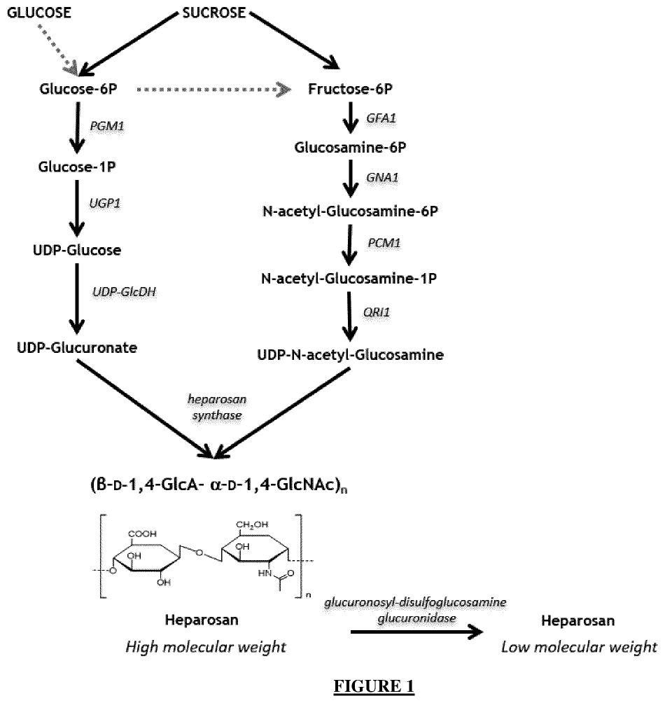

### SUMMARY OF THE SEQUENCES

SEQ ID NO: 1 is a reencoded nucleic acid sequence of heparosan synthase originating from Pasteurella multocida (HSS1)

SEQ ID NO: 2 is a reencoded nucleic acid sequence of heparosan synthase originating from Pasteurella multocida (HSS2) SEQ ID NO: 3 is an amino acid sequence of heparosan synthase originating from Pasteurella multocida (HSS1)

SEQ ID NO: 4 is an amino acid sequence of heparosan synthase originating from Pasteurella multocida (HSS2)

SEQ ID NO: 5 is a nucleic acid sequence of UDP-Glucose dehydrogenase (HASB) originating from Arabidopsis thaliana

SEQ ID NO: 6 is a reencoded nucleic acid sequence of UDP-Glucose dehydrogenase (HASB) originating from Chlorella virus PBCV-1

SEQ ID NO: 7 is a reencoded nucleic acid sequence of UDP-Glucose dehydrogenase (HASB-A) originating from Chlorella virus PBCV-1

SEQ ID NO: 8 is an amino acid sequence of UDP-Glucose dehydrogenase (HASB) originating from Arabidopsis thaliana

SEQ ID NO: 9 is an amino acid sequence of UDP-Glucose dehydrogenase (HASB) originating from Chlorella virus PBCV1

SEQ ID NO: 10 is a reencoded nucleic acid sequence of glucuronosyl-disulfoglucosamine glucuronidase originating from Pedobacter heparinus with a N-terminal secretion signal

SEQ ID NO: 11 is a reencoded nucleic acid sequence of glucuronosyl-disulfoglucosamine glucuronidase originating from Pedobacter heparinus with a N-terminal secretion signal and a C-terminal anchoring signal

SEQ ID NO: 12 is a reencoded nucleic acid sequence of glucuronosyl-disulfoglucosamine glucuronidase originating from Pedobacter heparinus with a N-terminal secretion signal

SEQ ID NO: 13 is a reencoded nucleic acid sequence of glucuronosyl-disulfoglucosamine glucuronidase originating from Pedobacter heparinus with a N-terminal secretion signal and a C-terminal anchoring signal

SEQ ID NO: 14 is an amino acid sequence of glucuronosyl-disulfoglucosamine glucuronidase originating from Pedobacter heparinus with a N-terminal secretion signal

SEQ ID NO: 15 is an amino acid sequence of glucuronosyl-disulfoglucosamine glucuronidase originating from Pedobacter heparinus with a N-terminal secretion signal and a C-terminal anchoring signal

SEQ ID NO: 16 is a nucleic acid sequence of glutamine-fructose-6-phosphate amidotransferase (GFA1) originating from Saccharomyces cerevisiae

SEQ ID NO: 17 is a reencoded nucleic acid sequence of glutamine-fructose-6-phosphate amidotransferase (GFA1) originating from Chlorella virus 1 (PBCV-1)

SEQ ID NO: 18 is a reencoded nucleic acid sequence of glutamine-fructose-6-phosphate amidotransferase (GFA1) originating from Chlorella virus 1 (PBCV-1)

SEQ ID NO: 19 is an amino acid sequence of glutamine-fructose-6-phosphate amidotransferase (GFA1) originating from Saccharomyces cerevisiae

SEQ ID NO: 20 is an amino acid sequence of glutamine-fructose-6-phosphate amidotransferase (GFA1) originating from Chlorella virus 1 (PBCV-1)

SEQ ID NO: 21 is a nucleic acid sequence of UDP-N-acetylglucosamine pyrophosphorylase (QRI1) originating from Saccharomyces cerevisiae

SEQ ID NO: 22 is an amino acid sequence of UDP-N-acetylglucosamine pyrophosphorylase (QRI1) originating from Saccharomyces cerevisiae

SEQ ID NO: 23 is a nucleic acid sequence of Phosphoglucomutase-1 (PGM1) originating from Saccharomyces cerevisiae

SEQ ID NO: 24 is an amino acid sequence Phosphoglucomutase-1 (PGM1) originating from Saccharomyces cerevisiae

SEQ ID NO: 25 is a nucleic acid sequence of UTP-glucose-1-phosphate uridylyltransferase (UGP1) originating from Saccharomyces cerevisiae

SEQ ID NO: 26 is an amino acid sequence of UTP-glucose-1-phosphate uridylyltransferase (UGP1) originating from Saccharomyces cerevisiae

SEQ ID NO: 27 is a nucleic acid sequence of Glucosamine 6-phosphate N-acetyltransferase

(GNA1) originating from Saccharomyces cerevisiae

SEQ ID NO: 28 is an amino acid sequence of Glucosamine 6-phosphate N-acetyltransferase (GNA1) originating from Saccharomyces cerevisiae

SEQ ID NO: 29 is a nucleic acid sequence of phosphoacetylglucosamine mutase (PCM1) originating from Saccharomyces cerevisiae

SEQ ID NO: 30 is an amino acid sequence of phosphoacetylglucosamine mutase (PCM1) originating from Saccharomyces cerevisiae

SEQ ID NO: 31 is a nucleic acid sequence of promotor pTDH3

SEQ ID NO: 32 is a nucleic acid sequence of promotor pTDH3.Sk

SEQ ID NO: 33 is a nucleic acid sequence of promotor pTDH3-1.sba

SEQ ID NO: 34 is a nucleic acid sequence of promotor pTDH3.Sar

SEQ ID NO: 35 is a nucleic acid sequence of promotor pENO2

SEQ ID NO: 36 is a nucleic acid sequence of promotor pTEF3

SEQ ID NO: 37 is a nucleic acid sequence of promotor pTEF1

SEQ ID NO: 38 is a nucleic acid sequence of promotor pTEF1.ago

SEQ ID NO: 39 is a nucleic acid sequence of promotor pTEF1.Sba

SEQ ID NO: 40 is a nucleic acid sequence of promotor pPDC1

SEQ ID NO: 41 is a nucleic acid sequence of promotor pCCW12

SEQ ID NO: 42 is a nucleic acid sequence of promotor pCCW12.Sm

SEQ ID NO: 43 is a nucleic acid sequence of promotor pCCW12.Sk

SEQ ID NO: 44 is a nucleic acid sequence of promotor pCCW12.Sba

SEQ ID NO: 45 is a nucleic acid sequence of promotor pCCW12.Sar

SEQ ID NO: 46 is a nucleic acid sequence of promotor pNUP57

SEQ ID NO: 47 is a nucleic acid sequence of promotor pCCW10.ago

SEQ ID NO: 48 is a nucleic acid sequence of promotor pCWP2

SEQ ID NO: 49 is a nucleic acid sequence of promotor pCCW120.Sca

SEQ ID NO: 50 is a nucleic acid sequence of promotor pRPLA1

SEQ ID NO: 51 is a nucleic acid sequence of promotor pCUP1

SEQ ID NO: 52 is a nucleic acid sequence of promotor pMET6

SEQ ID NO: 53 is a nucleic acid sequence of promotor pMET25

SEQ ID NO: 54 is a nucleic acid sequence of promotor pSAM1

SEQ ID NO: 55 is a nucleic acid sequence of terminator tTPI1

SEQ ID NO: 56 is a nucleic acid sequence of terminator tMET25

SEQ ID NO: 57 is a nucleic acid sequence of terminator tDIT1

SEQ ID NO: 58 is a nucleic acid sequence of terminator tRPL3

SEQ ID NO: 59 is a nucleic acid sequence of terminator tRPL3.sm

SEQ ID NO: 60 is a nucleic acid sequence of terminator tRPL3.sba

SEQ ID NO: 61 is a nucleic acid sequence of terminator tRPL41B

SEQ ID NO: 62 is a nucleic acid sequence of terminator tRPL15A

SEQ ID NO: 63 is a nucleic acid sequence of terminator tRPL15A.sba

SEQ ID NO: 64 is a nucleic acid sequence of terminator tIDP1

SEQ ID NO: 65 is a nucleic acid sequence of terminator tTEF1.sba

SEQ ID NO: 66 is a nucleic acid sequence of the secretion sequence added in 5′

SEQ ID NO: 67 is an amino acid sequence of the secretion sequence added in N-ter

SEQ ID NO: 68 is a nucleic acid sequence of the anchoring sequence added in 3′

SEQ ID NO: 69 is an amino acid sequence of the anchoring sequence added in C-ter

### DETAILED DESCRIPTION OF THE INVENTION

The inventors have conceived genetically modified cells, and especially genetically modified yeasts, having the ability to produce heparosan, as compared to the parent cells, and especially as compared to the parent yeasts which are not naturally capable of doing so.

These genetically modified cells are described throughout the present specification.

### Definitions

The term “heparosan” is a polysaccharide a polymer composed of [-4-N-acetylglucosamine-α1,4-glucuronic acid-β1-] repeats ([→4) B-D-glucuronic acid (GlcA) (1→4)N-acetyl-a-D-glucosamine (GlcNAc) (1→]ₙ).

Heparosan may be produced in a recombinant cell.

As used herein, the term “recombinant”, when used in reference to a cell, indicates that the cell has been modified by the introduction of an endogenous and/or heterologous nucleic acid or protein into the cell or the alteration of a native cell or that the cell is derived from a cell so modified. Thus, for example, recombinant cells express genes or nucleic acid that are not found within the native (non-recombinant) form of the cell or express native (eg endogenous) genes at a different level than their native level or express additional or supplementary copies of native (eg endogenous) at a different level than their native level.

As used herein, the term “recombinant”, when used in reference to a nucleic acid or vector, are sequences formed/obtained by technics of genetic engineering well known to the man skilled in the art. The guidelines of the US National Institute for Health (NIH) accordingly indicate that:
“recombinant [ . . . ] nucleic acids are defined as:
i. molecules that (a) are constructed by joining nucleic acid molecules and (b) that can replicate in a living cell, i.e., recombinant nucleic acids;”,
which reflects the conventional use of the word “recombinant” attached to nucleic acid sequences to mean recombined following the insertion of, or joining together with, another nucleic acid.

Proteins that result from the expression of recombinant DNA or recombinant vector within living cells are also termed recombinant proteins.

The term “recombinant” is accordingly synonymous with the term “genetically modified”. The term “gene” is synonymous with the term “nucleic acid” or “nucleotide sequence”.

A recombinant nucleic acid sequence for use in a recombinant cell, and in particular a recombinant yeast, of the invention may be provided in the form of a nucleic acid construct. The term “nucleic acid construct” refers to as a nucleic acid molecule, either single- or double-stranded, which is isolated or derived from a native (eg endogenous) naturally occurring gene or is a heterologous nucleic acid or which has been modified to contain segments of nucleic acid which are combined and juxtaposed in a manner which would not otherwise exist in nature. The term “nucleic acid construct” is synonymous with the term “expression cassette” or “heterologous nucleic acid expression cassette” when the nucleic acid construct contains one or more regulatory elements required for expression of a coding sequence, wherein said control sequences are operably linked to said coding sequence. Non-limiting examples of regulatory elements include promoters, enhancers, silencers, terminators, and poly-A signals.

A recombinant nucleic acid sequence for use in a recombinant cell of the invention may be provided in the form of an expression vector, wherein the polynucleotide sequence is operably linked to at least one control sequence for the expression of the polynucleotide sequence in a recombinant cell.

The terms “obtained from” or “originate from” or “originating from” a microorganism or animal generally means that a substance (e.g., a nucleic acid molecule or polypeptide) originating from the microorganism or animal is native to that microorganism or animal.

The terms “derived from” a microorganism or animal generally means that a substance (e.g., a nucleic acid molecule or polypeptide) derived from the microorganism or animal is the result of modifications brought to the substance native to (i.e. present as such) in that microorganism or animal. For example, regarding a nucleic acid sequence derived from a microorganism or animal, said nucleic acid sequence can correspond to a reencoded and/or truncated version of the native nucleic acid sequence from this microorganism or animal. Other modifications well known to the man skilled in the art can be brought to the native substance of a microorganism or animal leading to the substance used being “derived from” said microorganism or animal.

As used herein, the term “polypeptide” refers to a molecule comprising amino acid residues linked by peptide bonds and containing more than five amino acid residues. The amino acids are identified by either the single-letter or three-letter designations. The term “protein” as used herein is synonymous with the term “polypeptide” and may also refer to two or more polypeptides. Thus, the terms “protein”, “peptide” and “polypeptide” can be used interchangeably. Polypeptides may optionally be modified (e.g., glycosylated, phosphorylated, acylated, farnesylated, prenylated, sulfonated, and the like) to add functionality. Polypeptides exhibiting activity may be referred to as enzymes. It will be understood that, as a result of the degeneracy of the genetic code, a multitude of nucleotide sequences encoding a given polypeptide may be produced.

A polypeptide encoded by a recombinant nucleic acid for use in a recombinant cell, and in particular in a recombinant yeast, of the invention may comprise a signal peptide and/or a propeptide sequence. In the event that a polypeptide expressed by a recombinant cell, and in particular a recombinant yeast, of the invention comprises a signal peptide and/or a propeptide, sequence identity may be calculated over the mature polypeptide sequence.

The term “operably linked” as used herein refers to two or more nucleic acid sequence elements that are physically linked and are in a functional relationship with each other. For instance, a promoter is operably linked to a coding sequence if the promoter is able to initiate or regulate the transcription or expression of the coding sequence, in which case the coding sequence should be understood as being “under the control of” the promoter. Generally, when two nucleic acid sequences are operably linked, they will be in the same orientation and usually also in the same reading frame. They usually will be essentially contiguous, although this may not be required.

The term “native” or “endogenous” as used herein with reference to molecules, and in particular enzymes and nucleic acids, indicates molecules that are expressed in the organism in which they originated or are found in nature.

The term “endogenous gene” means that the gene was present in the cell before any genetic modification, in the wild-type strain. Endogenous genes may be overexpressed by introducing heterologous sequences in addition to, or to replace endogenous regulatory elements, or by introducing one or more additional or supplementary copies of the gene into the chromosome or a plasmid (said additional or supplementary copies being designated “exogenous or heterologous genes” or “heterologous nucleotide sequences” or “heterologous nucleic acids” as defined herein). Endogenous genes may also be modified to modulate their expression and/or activity. For example, mutations may be introduced into the coding sequence to modify the gene product or heterologous sequences may be introduced in addition to or to replace endogenous regulatory elements. Modulation of an endogenous gene may result in the up-regulation and/or enhancement of the activity of the gene product, or alternatively, in the down-regulation and/or attenuation of the activity of the endogenous gene product. Another way to enhance expression of endogenous genes is to introduce one or more additional or supplementary copies of the gene onto the chromosome or a plasmid (said supplementary copies being designated “exogenous or heterologous genes” or “heterologous nucleotide sequences” or heterologous nucleic acids” as defined herein).

By “one or more additional or supplementary copies of a gene” according to the invention, it is for example understood in the present invention from 1 to 50 copies, in particular from 1 to 30 copies, more particularly from 1 to 20 copies, and preferably from 1 to 10 copies. Said copies may be inserted into the same locus or into different loci of a recombinant cell of the invention.

The term “exogenous gene” means that the gene was introduced into a cell, by means well known to the man skilled in the art, whereas this gene is or is not naturally occurring in the wild-type cell. Cells can express exogenous genes if these genes are introduced into the cell with all the elements allowing their expression in the cell. Transforming cells with exogenous DNA is a routine task for the man skilled in the art. Exogenous genes may be integrated into the host chromosome, or be expressed extra-chromosomally from plasmids or vectors. A variety of plasmids, which differ with respect to their origin of replication and their copy number in the cell, are all known in the art. The sequence of exogenous genes may be adapted for its expression in the cell. Indeed, the man skilled in the art knows the notion of codon usage bias and how to adapt nucleic sequences for a particular codon usage bias without modifying the deduced protein. In particular embodiments, codon optimized genes express native enzymes.

The term “heterologous gene” or heterologous nucleic acid sequence” refers to a gene or nucleic acid sequence not normally found in a given cell in nature. As such, a heterologous nucleic acid sequence may be: (a) foreign to its host cell (i.e. is “exogenous” to the cell); (b) naturally found in the host cell (i.e. “endogenous”) but present at an unnatural quantity in the cell (i.e., greater or lesser quantity than naturally found in the host cell); or (c) be naturally found in the host cell but positioned outside of its natural locus.

In the present application, all genes are referenced with their common names and with references to their nucleotide sequences and, the case arising, to their amino acid sequences. Using the references given in accession number for known genes, those skilled in the art are able to determine the equivalent genes in other organisms, bacterial strains, yeast, fungi, mammals, plants, etc. This routine work is advantageously done using consensus sequences that can be determined by carrying out sequence alignments with genes derived from other cells and designing degenerated probes to clone the corresponding gene in another organism.

The man skilled in the art knows different means to modulate, and in particular up-regulate or down-regulate, the expression of endogenous genes. For example, a way to enhance expression of, or over express, endogenous genes is to introduce one or more additional or supplementary copies of the gene onto the chromosome or a plasmid.

Another way is to replace the endogenous promoter of a gene with a stronger promoter. These promoters may be homologous or heterologous. Promoters particularly interesting in the present invention are described in more detail elsewhere in the present specification.

The nucleic acid expression construct may further comprise 5′ and/or 3′ recognition sequences and/or selection markers.

The term “inducible promoter” is used to qualify a promoter whose activity is induced, i.e. increased:
in the presence of one or more particular metabolite(s). The higher the metabolite concentration in the medium, the stronger the promoter activity; or
in the presence of a low concentration, or in the absence, of one or more metabolite(s). These metabolites are different from those whose increasing presence induces the activity of the promoter. The lower the metabolite concentration in the medium, the stronger the promoter activity.

The term “repressible promoter” is used to qualify a promoter whose activity is repressed, i.e. reduced:
in the presence of one or more particular metabolite(s). The higher the metabolite concentration in the medium, the weaker the promoter activity; or
in the presence of a low concentration, or in the absence, of one or more metabolite(s). These metabolites are different from those whose increasing presence represses the activity of the promoter. The lower the metabolite concentration in the medium, the weaker the promoter activity.

As used herein, the term “anchoring signal” when used in conjunction with a protein or polypeptide such as an enzyme (such as, for example a polypeptide having a glucuronosyl-disulfoglucosamine glucuronidase activity) means for example a first nucleic acid encoding a protein that is operably linked to a second nucleic acid encoding a protein or a polypeptide, or a first protein or polypeptide that is operably linked to a second protein or polypeptide, such as an enzyme (such as, for example a polypeptide having a glucuronosyl-disulfoglucosamine glucuronidase activity to form, for example a fusion protein), and that enables the cellular transport machinery of a cell, in particular of a S. cerevisiae cell, to correctly anchor and/or position in the membrane of the cell the second protein operably linked to the first protein.

As used herein, the term “secretion signal” when used in conjunction with a protein or polypeptide such as an enzyme (such as, for example a polypeptide having a glucuronosyl-disulfoglucosamine glucuronidase activity) means for example a first nucleic acid encoding a peptide or protein that is operably linked to a second nucleic acid encoding a protein, or a first protein that is linked to a second protein, such as an enzyme (such as, for example a polypeptide having a glucuronosyl-disulfoglucosamine glucuronidase activity to form, for example a fusion protein), and that enables the cellular transport machinery of a cell, in particular of a S. cerevisiae cell, to locate at least the second protein to the membrane of the cell and to secrete the second protein outside of the cell, after the first protein has, for example, been cleaved from the second protein.

As used herein the terms “secretion signal” and “anchoring signal” when used in conjunction with a protein or polypeptide such as an enzyme (such as, for example, a polypeptide having a glucuronosyl-disulfoglucosamine glucuronidase activity), mean for example a first nucleic acid encoding a peptide or protein that is operably linked to a second nucleic acid encoding a protein, or a first protein that is operably linked to a second protein, such as an enzyme (such as, for example a polypeptide having a glucuronosyl-disulfoglucosamine glucuronidase activity), and that enables the cellular transport machinery of a cell, in particular of a S. cerevisiae cell, to locate at least the second protein to the membrane of the cell wherein if the second protein is also operably linked to an “anchoring signal” the second protein is not secreted but remains attached to the membrane of the cell. In some cases, a secretion-anchoring signal may provide a dual secretion signal and anchoring signal function.

Sequences of secretion and anchoring signals, methods for the expression, anchoring and/or secretion of heterologous proteins, such as enzymes (such as, for example, glucuronosyl-disulfoglucosamine glucuronidase) on the surface of a cell (such, as for example, a yeast cell) are well known in the art (see for example, Ast et al (2013) Cell 152:1134-1145, Ast and Schuldiner (2013) Crit Rev Biochem Mol Biol 48 (3) 273-288, Van der Vaart et al (1997) Applied Environmental Microbiology 63 (2) 615-620 and the entire contents of each of these publications are incorporated herein by reference).

The “activity” of an enzyme is used interchangeably with the term “function” and designates, in the context of the invention, the capacity of an enzyme to catalyze a desired reaction. The amount of an enzyme in a host cell may be altered by modifying the transcription of the gene that encodes the enzyme. This can be achieved for example by modifying the copy number of the nucleotide sequence encoding the enzyme (e.g., by using a higher or lower copy number expression vector comprising the nucleotide sequence, or by introducing additional copies of the nucleotide sequence into the genome of the host cell or by deleting or disrupting the nucleotide sequence in the genome of the host cell), by changing the order of coding sequences on a polycistronic mRNA of an operon or breaking up an operon into individual genes each with its own control elements, or by increasing the strength of the promoter or operator to which the nucleotide sequence is operably linked.

Alternatively or in addition, the copy number of an enzyme in a host cell may be altered by modifying the level of translation of an mRNA that encodes the enzyme. This can be achieved for example by modifying the stability of the mRNA, modifying the sequence of the ribosome binding site, modifying the distance or sequence between the ribosome binding site and the start codon of the enzyme coding sequence, modifying the entire intercistronic region located “upstream of” or adjacent to the 5′ side of the start codon of the enzyme coding region, stabilizing the 3′-end of the mRNA transcript using hairpins and specialized sequences, modifying the codon usage of enzyme, altering expression of rare codon tRNAs used in the biosynthesis of the enzyme, and/or increasing the stability of the enzyme, as, for example, via mutation of its coding sequence.

The activity of an enzyme in a host cell can be altered in a number of ways, including, but not limited to, expressing a modified form of the enzyme that exhibits increased or decreased solubility in the host cell, expressing an altered form of the enzyme that lacks a domain through which the activity of the enzyme is inhibited, expressing a modified form of the enzyme that has a higher or lower Kcat or a lower or higher Km for the substrate, or expressing an altered form of the enzyme that is more or less affected by feed back or feed-forward regulation by another molecule in the pathway.

The terms “encoding” or “coding for” refer to the process by which a polynucleotide, through the mechanisms of transcription and translation, produces an amino-acid sequence.

The gene(s) encoding the enzyme(s) considered in the present invention can be exogenous or endogenous.

The methods implemented in the present invention preferably require the use of one or more chromosomal integration constructs for the stable introduction of a heterologous nucleotide sequence into a specific location on a chromosome or for the functional disruption of one or more target genes in a genetically modified cell. In some embodiments, disruption of a target gene prevents the expression of the related functional protein. In some embodiments, disruption of a target gene results in the expression of a non-functional protein from the disrupted gene.

Parameters of chromosomal integration constructs that may be varied in the practice of the present invention include, but are not limited to, the lengths of the homologous sequences; the nucleotide sequence of the homologous sequences; the length of the integrating sequence; the nucleotide sequence of the integrating sequence; and the nucleotide sequence of the target locus. In some embodiments, an effective range for the length of each homologous sequence is 20 to 5,000 base pairs, preferentially 50 to 100 base pairs. In particular embodiments, the length of each homologous sequence is about 50 base pairs. For more information on the length of homology required for gene targeting, see D. Burke et al., Methods in yeast Genetics—A cold spring harbor laboratory course Manual (2000).

In some embodiments, (a) disrupted gene(s) in which the above-mentioned DNA construct(s) is/are intended to be inserted may advantageously comprises one or more selectable markers useful for the selection of transformed cells. Preferably, said selectable marker(s) are comprised in the DNA construct(s) according to the present invention.

In some embodiments, the selectable marker is an antibiotic resistance marker. Illustrative examples of antibiotic resistance markers include, but are not limited to the, NATI, AURI-C, HPH, DSDA, KAN<R>, and SH BLE gene products. The NAT 1 gene product from S. noursei confers resistance to nourscothricin; the AURI-C gene product from Saccharomyces cerevisiae confers resistance to Aucrobasidin A (AbA); the HPH gene product of Klebsiella pneumoniae confers resistance to Hygromycin B; the DSDA gene product of E. coli allows cells to grow on plates with D-serine as the sole nitrogen source; the KAN<R> gene of the Tn903 transposon confers resistance to G418; and the SH BLE gene product from Streptoalloteichus hindustanus confers resistance to Zeocin (bleomycin).

In some embodiments, the antibiotic resistance marker is deleted after the genetically modified cell of the invention is isolated. The man skilled in the art is able to choose suitable marker in specific genetic context.

In a particular embodiment, a recombinant cell according to the invention is devoid of any antibiotic resistance marker. This advantageously prevents the necessity to add antibiotics in the selection medium.

In some embodiments, the selectable marker rescues an auxotrophy (e.g., a nutritional auxotrophy) in the genetically modified cell. In such embodiments, a parent cell, and in particular a parent yeast, comprises a functional disruption in one or more gene products that function in an amino acid or nucleotide biosynthetic pathway, such as, for example, the HIS3, LEU2, LYS1, LYS2, MET15, TRP1, ADE2, and URA3 gene products in yeast, which renders the parent cell incapable of growing in media without supplementation with one or more nutrients (auxotrophic phenotype). The auxotrophic phenotype can then be rescued by transforming the parent cell with a chromosomal integration encoding a functional copy of the disrupted gene product (in some embodiments the functional copy of the gene may originate from close species, such as Kluveromyces, Candida etc.), and the genetically modified cell generated can be selected based on the loss of the auxotrophic phenotype of the parent microbial cell.

For each of the nucleic acid sequences comprising a promoter sequence, a coding sequence (e.g. an enzyme coding sequence), or a terminator sequence, reference sequences are described herein. The present description also encompasses nucleic acid sequences having specific percentages of nucleic acid identity with a reference nucleic acid sequence.

For each or the amino acid sequences of interest, reference sequences are described herein. The present description also encompasses amino acid sequences (e.g. enzyme amino acid sequences), having specific percentages of amino acid identity with a reference amino acid sequence.

For obvious reasons, in all the present description, a specific nucleic acid sequence or a specific amino acid sequence which complies with, respectively, the considered nucleotide or amino acid identity, should further lead to obtaining a protein (or enzyme) which displays the desired biological activity. As used herein, the “percentage of identity” between two nucleic acid sequences or between two amino acid sequences is determined by comparing both optimally aligned sequences through a comparison window.

The portion of the nucleotide or amino-acid sequence in the comparison window may thus include additions or deletions (for example “gaps”) as compared to the reference sequence (which does not include these additions or these deletions) so as to obtain an optimal alignment between both sequences.

The terms “sequence homology” or “sequence identity” or “homology” or “identity” are used interchangeably herein. For the purpose of the invention, it is defined here that in order to determine the percentage of sequence homology or sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley).

The percent sequence identity between two amino acid sequences or between two nucleotide sequences may be determined using the Needleman and Wunsch algorithm for the alignment of two sequences. (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE.

For the purpose of the invention, the NEEDLE program from the EMBOSS package was used (version 2.8.0 or higher, EMBOSS: The European Molecular Biology Open Software Suite (2000) Rice, P. Longden J. and Bleasby, A. Trends in Genetics 16, (6) pp 276 277, http://emboss.bioinformatics.nl/). For protein sequences EBLOSUM62 is used for the substitution matrix. For nucleotide sequence, EDNAFULL is used. The optional parameters used are a gap opening penalty of 10 and a gap extension penalty of 0.5. No end gap penalty is added. In the Output section, Yes has been indicated in response to the question “Brief identity and similarity” and “SRS pairwise” indicated as Output alignment format.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment. The identity defined as herein can be obtained from NEEDLE by using the NOBRIEF option and is labeled in the output of the program as “longest-identity”.

The similarity of nucleotide and amino acid sequences, i.e. the percentage of sequence identity, can be determined via sequence alignments using several other art-known algorithms, preferably with the mathematical algorithm of Karlin and Altschul (Karlin & Altschul (1993) Proc. Natl. Acad. Sci. USA 90:5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) available e.g. on https://www.ebi.ac.uk/Tools/msa/clustalo/or the GAP program (mathematical algorithm of the University of Iowa) or the mathematical algorithm of Myers and Miller (1989-Cabios 4:11-17) or Clone Manager 9. Preferred parameters used are the default parameters as they are set on https://www.ebi.ac.uk/Tools/msa/clustalo/.

The grade of sequence identity (sequence matching) may be calculated using e.g. BLAST, BLAT or BlastZ (or BlastX). A similar algorithm is incorporated into the BLASTN and BLASTP programs of Altschul et al (1990) J. Mol. Biol. 215, 403-410. BLAST polynucleotide searches are performed with the BLASTN program, score=100, word length=12, to obtain polynucleotide sequences that are homologous to those nucleic acids which encode the relevant protein.

BLAST protein searches are performed with the BLASTP program, score=50, word length=3, to obtain amino acid sequences homologous to the SHC polypeptide. To obtain gapped alignments for comparative purposes, Gapped BLAST is utilized as described in Altschul et al (1997) Nucleic Acids Res. 25, 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields. When percentages of sequence identity are referred to in the present application, these percentages are calculated in relation to the full length of the longer sequence, if not specifically indicated otherwise.

In particular embodiments, % identity between two sequences is determined using CLUSTAL O (version 1.2.4).

The “fermentation” or “culture” is generally conducted in fermenters with an appropriate culture medium adapted to the cell being cultivated, containing at least one simple carbon source, and if necessary co-substrates.

The term “fermentation composition” refers to a composition which comprises genetically modified host cells and products or metabolites produced by the genetically modified host cells. An example of a fermentation composition is a whole cell broth, which can be the entire contents of a vessel (e.g., a flasks, plate, or fermentor), including cells, aqueous phase, and compounds produced from the genetically modified host cells.

The term “medium” refers to a culture medium or cultivation medium or fermentation medium.

For maximal production of heparosan, the recombinant cells used as production hosts preferably have a high rate of carbohydrate utilization. These characteristics may be conferred by mutagenesis and selection, genetic engineering, or may be natural. Fermentation media, or “culture medium” or “cultivation medium”, for the present cells may contain at least about 10 g/L of glucose and/or sucrose. Additional carbon substrates may include but are not limited to monosaccharides such as fructose, mannose, xylose and arabinose; oligosaccharides such as lactose, maltose, galactose, or sucrose; polysaccharides such as starch or cellulose or mixtures thereof and unpurified mixtures from renewable feedstocks such as cheese whey permeate cornsteep liquor, sugar beet molasses, and barley malt. Other carbon substrates may include glycerol, acetate and/or ethanol.

Hence, it is contemplated that the source of carbon utilized in the present invention may encompass a wide variety of carbon containing substrates and will only be limited by the choice of cell, and in particular of yeast.

Although it is contemplated that all of the above-mentioned carbon substrates and mixtures thereof are suitable in the present invention, preferred carbon substrates are glucose, fructose, and sucrose, or mixtures of these with C5 sugars such as xylose and/or arabinose for cells, and in particular yeasts, modified to use C5 sugars, and more particularly glucose.

Preferred carbon substrates are glucose or sucrose.

In addition to an appropriate carbon source, fermentation media may contain suitable minerals, salts, cofactors, buffers and other components, known to those skilled in the art, suitable for the growth of the cultures and promotion of the enzymatic pathway necessary for the production of the desired product.

Besides, additional genetic modifications suitable for the growth of recombinant cells according to the invention may be considered.

The terms “Aerobic conditions” refers to concentrations of oxygen in the culture medium that are sufficient for an aerobic or facultative anaerobic cell, and in particular yeast, to use di-oxygene as a terminal electron acceptor.

“Microaerobic condition” refers to a culture medium in which the concentration of oxygen is less than that in air, i.e. oxygen concentration up to 6% O₂.

An “appropriate culture medium” designates a medium (e.g. a sterile, liquid medium) comprising nutrients essential or beneficial to the maintenance and/or growth of the cell such as carbon sources or carbon substrate, nitrogen sources, for example, peptone, yeast extracts, meat extracts, malt extracts, urea, ammonium sulfate, ammonium chloride, ammonium nitrate and ammonium phosphate; phosphorus sources, for example, monopotassium phosphate or dipotassium phosphate; trace elements (e.g., metal salts), for example magnesium salts, cobalt salts and/or manganese salts; as well as growth factors such as amino acids, vitamins, growth promoters, and the like. The term “carbon source” or “carbon substrate” or “source of carbon” according to the present invention denotes any source of carbon that can be used by those skilled in the art to support the normal growth of a cell, including hexoses (such as glucose, galactose or lactose), pentoses, monosaccharides, oligosaccharides, disaccharides (such as sucrose, cellobiose or maltose), molasses, starch or its derivatives, cellulose, hemicelluloses and combinations thereof.

Culture mediums that are particularly appropriate to produce recombinant cells of the invention, and in particular recombinant yeasts of the invention, are described in greater details further below in the text.

By “controlled” molecular weight of a heparosan of the invention is intended to mean that at least 80%, in particular at least 85%, of the heparosan produced by a method of the invention has a molecular weight comprised within a pre-determined molecular weight's range through the regulation of at least one parameter of the method and/or of the recombinant cell of the invention, such as for example:
the nature and origin of the nucleic acid encoding a polypeptide having glucuronosyl-disulfoglucosamine glucuronidase activity of the recombinant cell,
the nature and origin of the promoter controlling the expression of the nucleic acid encoding the polypeptide having glucuronosyl-disulfoglucosamine glucuronidase activity of the recombinant cell,
the presence of an anchoring and/or of a secretion signal associated to the encoded polypeptide having glucuronosyl-disulfoglucosamine glucuronidase activity of the recombinant cell,
the pH of the culture medium during the step of culturing the recombinant cell, and/or
the duration of the culturing of the recombinant cell.

As used herein, the term “about” refers to a reasonable range about a value as determined by the practitioner of skill. In certain embodiments, the term about refers to +one, two, or three standard deviations. In certain embodiments, the term about refers to +5%, 10%, 20%, or 25%. In certain embodiments, the term about refers to +0.1, 0.2, or 0.3 logarithmic units, e.g. pH units.

### General Features of Genetic Modifications Introduced According to the Invention

All the genome modifications are inserted in recombinant cells, and in particular recombinant yeasts, according to known genetic engineering techniques:
The successive nucleic acid sequences included in a gene construct that is introduced in the recombinant cell genome according to the invention are of the following structure:

Prom₁-ORF₁-term₁-ORF₂-gene₂-therm₂- . . . / . . . Promₙ-ORFₙ-termₙ, wherein:
Prom₁ is a sequence regulating the expression of the coding sequence ORF1,
ORF1 is a nucleic acid sequence encoding a desired protein PROT1, and especially a desired enzyme PROT1,
Term1 is a transcription terminator sequence that mediates transcriptional termination by providing signals in the newly synthesized mRNA that trigger processes which release the mRNA from the transcriptional complex, and
“1”, “2”, . . . / . . . “n” may or may not describe the same ORF (Open Reading Frame), promoter or terminator. The order of the nucleic acid sequences does not matter. “n” is an integer usually ranging from 5 and 20. These constructs are inserted in one of the recombinant cell chromosome at a controlled location. In some embodiments, the insertion site is neither essential for the functionality of the inserted construct, nor for the viability of the resulting genetically modified cell.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.” Codon optimization for other host cells can be readily determined using codon usage tables or can be performed using commercially available software, such as CodonOp (www.idtdna.com/CodonOptfrom) from Integrated DNA Technologies. Optimized coding sequences containing codons preferred by a particular prokaryotic or eukaryotic host (Murray et al, 1989, Nucl Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24:216-8).
When the recombinant cell is a yeast cell, and in particular is Saccharomyces cerevisiae yeast cell, nucleic acid sequences introduced in the yeast genome and originating from other organisms than Saccharomyces cerevisiae are generally “transcoded” (generally “codon-optimized”), meaning that these nucleic acid sequences are synthesized with an optimal codon usage for expression in S. cerevisiae. The nucleotide sequence (and not the protein sequence) of some nucleic acid sequences from S. cerevisiae has also been modified (“transcoded”) to minimize recombination with an endogenous copy of the said gene.
Genes may be deleted through standard procedures used in cell genetic engineering. In some embodiments, the genes targeted for deletion may be interrupted by insertion of one of the above described gene constructs, or alternatively the genes targeted for deletion are replaced by a short stretch of nucleotide.
A nucleic acid sequences may be rendered “inducible or repressible” by deleting an endogenous copy of the nucleic acid sequences (if necessary) and placing a new copy of the ORF under the control of an inducible or repressible promoter. An inducible or repressible promoter is a promoter which activity is modulated or controlled, i.e. either increased or decreased, upon a change in the environmental conditions or external stimuli. Induction or repression may be artificially controlled, which encompasses induction or repression by abiotic factors such as chemical compounds not found naturally in the cell, and in particular yeast, of interest, light, oxygen levels, heat or cold. A list and sequences of inducible or repressible promoters are described elsewhere in the present specification.

### Recombinant Cells According to the Invention

The inventors have conceived recombinant cells, and especially recombinant yeasts, having an ability of producing heparosan.

The present invention relates to recombinant cells, and in particular recombinant yeasts, having the ability to produce heparosan, and wherein this ability to produce heparosan is obtained through a plurality of alterations that have been introduced in the genome thereof, by genetic engineering methods.

This present invention pertains to a recombinant yeast cell producing heparosan, wherein the recombinant cell comprises:
(a) one or more recombinant nucleic acids encoding a polypeptide having a heparosan synthase (HSS) activity; and
(b) one or more recombinant nucleic acids encoding a polypeptide having a UDP-Glucose dehydrogenase (UDP-GlcDH or HASB) activity.

The present invention further relates to a recombinant host cell producing heparosan, wherein the recombinant cell comprises:
(a) one or more recombinant nucleic acids encoding a polypeptide having a heparosan synthase (HSS) activity;
(b) one or more recombinant nucleic acids encoding a polypeptide having a UDP-Glucose dehydrogenase (UDP-GlcDH or HASB) activity; and
(c) one or more recombinant nucleic acids encoding a polypeptide having a glucuronosyl-disulfoglucosamine glucuronidase activity,
wherein the polypeptide having a glucuronosyl-disulfoglucosamine glucuronidase activity comprises a secretion signal, and optionally an anchoring signal, so that heparosan, in particular of a desired molecular weight, is produced by the host cell.

The inventors have found that an ability to produce heparosan by cells, and in particular yeast cells, may be reached by introducing in the genome of these cells a plurality of genetic alterations.

The production of heparosan by cells of the invention, and in particular by yeast cells of the invention, achieved by optimizing the endogenous metabolism of UDP-Glucose, and optionally UDP-N-Acetyl-glucosamine, and direct the subsequent artificially modified metabolic pathway mainly towards heparosan production while in the same time maintaining an optimal viability of the resulting genetically modified cells.

It has been determined that a heparosan production by recombinant cells according to the invention may be increased by increasing the conversion of glucose-6-phosphate into the successive intermediate metabolites (i) glucose-1-phosphate, UDP-glucose, UDP-glucuronate and heparosan and (ii) fructose-6-phosphate, glucosamine-6-phosphate, N-acetyl-glucosamine-6-phosphate, N-acetyl-glucosamine-1-phosphate, UDP-N-acetyl-glucosamine and heparosan, while maintaining a balance allowing a good viability of the resulting recombinant cells.

Indeed, in order to obtain a viable recombinant cell of the invention, many different constructs were tested in order to obtain a viable and efficient recombinant cell, and in particular a viable recombinant yeast. In particular such recombinant yeast were difficult to obtain because the temporary accumulation of some intermediates appeared to be toxic for yeasts.

Unexpected technical difficulties were encountered in establishing the conditions suitable for the preparation of a recombinant cell able to produce heparosan, and in particular to produce heparosan with a controlled molecular weight.

By “controlled” molecular weight of a heparosan of the invention is intended to mean that at least 80%, in particular at least 85%, of the heparosan produced by a method of the invention has a molecular weight comprised within a certain molecular weight's range through the regulation of at least one parameter of the method and/or of the recombinant cell of the invention, such as for example:
the nature and origin of the nucleic acid encoding polypeptide having a glucuronosyl-disulfoglucosamine glucuronidase activity of the recombinant cell,
the nature and origin of the promoter controlling the expression of the nucleic acid encoding the polypeptide having a glucuronosyl-disulfoglucosamine glucuronidase activity of the recombinant cell,
the presence of an anchoring and/or of a secretion signal associated to the polypeptide having a glucuronosyl-disulfoglucosamine glucuronidase activity of the recombinant cell,
the pH of the culture medium during the step of culturing the recombinant cell, and/or
the duration of the culturing of the recombinant cell.

Indeed, after much research and experimental trial, the inventors discovered that it was possible to cultivate a recombinant cell, and in particular a recombinant yeast cell, more particularly a recombinant Saccharomyces cerevisiae yeast cell, able to produce heparosan having a controlled molecular weight controlled using the following parameters:
the selection of the nature and origin of the nucleic acid sequences encoding the polypeptide having glucuronosyl-disulfoglucosamine glucuronidase activity of the recombinant cell, in particular the recombinant yeast, of the invention; and/or
the nature and origin of the promoter controlling the expression of the nucleic acid sequences encoding the polypeptide having glucuronosyl-disulfoglucosamine glucuronidase activity of the recombinant cell, in particular the recombinant yeast, of the invention; and/or
the optional presence of an anchoring signal, in addition to a secretion signal, associated to the encoded polypeptide having glucuronosyl-disulfoglucosamine glucuronidase activity of the recombinant cell, in particular the recombinant yeast, according to the invention; and/or
the pH of the culture medium during the step of culturing the recombinant cell, in particular the recombinant yeast cell, according to the invention; and/or
the duration of the culturing of the recombinant cell, in particular the recombinant yeast cell, according to the invention.

To the inventors' knowledge, this has never been achieved before.

The nucleic acid encoding a polypeptide having glucuronosyl-disulfoglucosamine glucuronidase activity may be obtained or derived from Pedobacter heparinus.

The nucleic acid encoding a polypeptide having a glucuronosyl-disulfoglucosamine glucuronidase activity may be under the control of a promoter selected from the group consisting of pTEF1, pCCW12, pCCW12.sba, pCCW12.Sar, pPDC1, pTEF3, pTDH3, pNUP57 and pCCW10.ago.

The molecular weight of the heparosan can be in the range of less than 50 kDa, preferably in the range of about 20 kDa to about 50 kDa.

Alternatively, the molecular weight of the heparosan can be in the range of greater than 50 kDa, preferably in the range of about 50 kDa to about 250 kD.

In another alternative, the molecular weight of the heparosan can be in the range of greater than 100 kDa, preferably in the range of about 100 kDa to about 1500 kDa.

The nucleic acid encoding a polypeptide having a UDP-Glucose dehydrogenase (UDP-GlcDH or HASB) activity may be obtained or derived from at least one of Arabidopsis thaliana, Chlorella virus PBCV1 or Streptococcus zooepidemicus, and may in particular be from Arabidopsis thaliana or Chlorella virus PBCV1.

The nucleic acid encoding a polypeptide having a heparosan synthase (HSS) activity may be obtained or derived from Pasteurella multocida.

The recombinant cell according to the invention can further comprise at least one recombinant nucleic acid encoding one or more of:
(i) a polypeptide having a glutamine-fructose-6-phosphate amidotransferase (GFA1) activity; and/or
(ii) a polypeptide having a UDP-N-acetylglucosamine pyrophosphorylase (QRI1) activity.

The recombinant cell according to the invention can further comprise at least one recombinant nucleic acid encoding one or more of:
(i) a polypeptide having Phosphoglucomutase-1 (PGM1) activity; and/or
(ii) a polypeptide having UTP-glucose-1-phosphate uridylyltransferase (UGP1) activity; and/or
(iii) a polypeptide having Glucosamine-6-phosphate N-acetyltransferase (GNA1) activity; and/or
(iv) a polypeptide having phosphoacetylglucosamine mutase (PCM1) activity.

The recombinant cell according to the invention can further be such that:
the nucleic acid encoding the polypeptide having glutamine-fructose-6-phosphate amidotransferase (GFA1) activity; and/or
the nucleic acid encoding the polypeptide having UDP-N-acetylglucosamine pyrophosphorylase (QRI1) activity; and/or
the nucleic acid encoding the polypeptide having Phosphoglucomutase-1 (PGM1) activity; and/or
the nucleic acid encoding the polypeptide having UTP-glucose-1-phosphate uridylyltransferase (UGP1) activity; and/or
the nucleic acid encoding the polypeptide having Glucosamine-6-phosphate N-acetyltransferase (GNA1) activity; and/or
the nucleic acid encoding the polypeptide having phosphoacetylglucosamine mutase (PCM1);
are obtained or derived from Saccharomyces cerevisiae.

A recombinant host cell of the invention may in particular be a yeast.

The recombinant cell of the invention may be a yeast cell belonging to the Saccharomyces genus, or to the Candida genus, or to the Kluyveromyces genus, or to the Ogataea genus, or to the Yarrowia genus, or to the Debaryomyces genus, or to the Ashbya genus, and in particular may belong to the Saccharomyces genus, and may more particularly be selected from the group consisting of Saccharomyces cerevisiae, Saccharomyces boulardii, Saccharomyces bayanus, Saccharomyces paradoxus, Saccharomyces mikatae, Saccharomyces castelli, Candida albicans, Candida glabrata, Candida tropicalis, Kluyveromyces lactis, Kluyveromyces marxianus, Kluyveromyces polysporus, Kluyveromyces thermotolerens, Ogataea polymorpha, Yarrowia lypolytica, Debaryomyces hansenii, and Ashbya gossypii, and may preferably be Saccharomyces cerevisiae.

Another object of the present invention relates to a method of producing heparosan of a desired molecular weight comprising:
(a) cultivating a recombinant cell of the invention, i.e. a recombinant yeast cell or a recombinant host cell of the invention, in a cultivation medium for a time sufficient to produce heparosan of the desired molecular weight; and
(b) optionally isolating or recovering the heparosan from the recombinant cell and/or from the cultivation medium.

In the method of the invention, the heparosan may have a molecular weight of from about 20 kDa to about 50 kDa, and preferably from 20 kDa to 30 kDa.

In the method of the invention, the heparosan may alternatively have a molecular weight of from 30 kDa to 50 kDa.

In the method of the invention, the heparosan may alternatively have a molecular weight of from about 50 kDa to about 150 kDa.

In the method of the invention, the heparosan may alternatively have a molecular weight of from about 150 kDa to about 1500 kDa.

In a method of the invention, the recombinant cell may be a yeast, in particular a member of the genus Saccharomyces genus, and may in particular be Saccharomyces cerevisiae.

In the method of the invention, the time sufficient to produce heparosan, in particular of a desired molecular weight, may be a period of from about 35 hours to about 50 hours, preferably from about 40 hours to about 50 hours, preferably of about 48 hours.

In the method of the invention, the molecular weight of the produced heparosan may be controlled by the pH of the cultivation medium.

In the method of the invention, the molecular weight of the heparosan may be controlled by regulating the pH of the cultivation medium during the cultivation step (a) of a method of the invention.

In the method of the invention, the molecular weight of the heparosan may be controlled by removing the biomass from the cultivation medium.

The method of the invention can be carried out on an industrial scale, preferably where the cultivation medium is at least about 100 L, more preferably in the range of about 1000 L to about 3000 L, even more preferably about 10,000 L or even more preferably about 100,000 L, or even about 250,000 L.

Another object of the invention relates to heparosan obtained or obtainable from a recombinant cell of the invention or from the method of the invention.

The present invention further relates to a cultivation medium comprising the heparosan according to the invention.

The present invention further relates to a composition comprising the heparosan according to the invention.

The present invention moreover relates to an industrial product or a consumer product or a consumable comprising (i) the heparosan having a molecular weight of less than 50 kDa, preferably in the range of about 20 kDa to about 50 kDa; or greater than 50 kDa, preferably in the range of about 50 kDa to about 250 kD; or greater than 100 kDa, preferably in the range of about 100 kDa to about 1500 kDa; (ii) the cultivation medium of the invention or (iii) the composition of the invention.

The industrial product or the consumer product or the consumable of the invention can be a cosmetic product, a flavour product, a fragrance product, a food product, a food, a beverage, a texturant, a pharmaceutical composition, a dietary supplement, a nutraceutical, a cleaning product, a dental and/or an oral hygiene composition.

Another object of the invention relates to the use of a recombinant cell of the invention for the production of heparosan having a molecular weight in the range of from about 20 kDa to about 50 kDa or from about 50 kDa to about 1000 kDa.

### Recombinant Nucleic Acids Encoding a Polypeptide Having Heparosan Synthase Activity

A recombinant cell according to the invention, and in particular a recombinant yeast of the invention, comprises one or more recombinant nucleic acids encoding a polypeptide having heparosan synthase activity.

A polypeptide having heparosan synthase activity according to the invention means a polypeptide that converts the intermediate metabolites UDP-Glucoronate and UDP-N-acetylglucosamine (UDP-GlcNAc) into heparosan [→4) β-D-glucuronic acid (GlcA) (1→4)N-acetyl-a-D-glucosamine (GlcNAc) (1→]ₙ.

In an embodiment, one or more of the recombinant nucleic acids encoding a polypeptide having heparosan synthase activity are under the control of an inducible or repressible promoter that is functional in the recombinant cells of the invention.

One or more of the recombinant nucleic acids encoding a polypeptide having heparosan synthase activity may be under the control of a promoter selected from the group consisting of pCCW12.Sar, pCCW120.sca, pTDH3-1.Sba and pTDH3.Sar.

The said one or more recombinant nucleic acids encoding a polypeptide having heparosan synthase activity may originate or be derived from Streptococcus zooepidemicus (sz), Chlorella virus PBCV1 (Vir), Xenopus laevis (xl) or Pasteurella multocida (pm), and in particular from Pasteurella multocida, as shown in the examples herein.

A recombinant cell according to the invention, and in particular a recombinant yeast according to the invention, may comprise between 1 and 5, in particular between 1 and 3 recombinant nucleic acids encoding a polypeptide having heparosan synthase activity. A recombinant cell according to the invention, and in particular a recombinant yeast according to the invention, may for example comprise 1, 2 or 3 recombinant nucleic acids encoding a polypeptide having heparosan synthase activity.

Illustratively, the one or more recombinant nucleic acids encoding a polypeptide having heparosan synthase activity may be inserted within the JLP1 gene and/or within the LEU2 gene and/or within the SAM3 gene of the recombinant cell, and in particular of the recombinant yeast, as it is shown in the examples herein.

In an embodiment of the invention, a recombinant cell, and in particular a recombinant yeast, of the invention comprises:
between 1 and 5, in particular between 1 and 3 recombinant nucleic acids encoding a polypeptide having heparosan synthase activity;
said one or more recombinant nucleic acids encoding a polypeptide having heparosan synthase activity originating or being derived from Streptococcus zooepidemicus (sz), Chlorella virus PBCV1 (Vir), Xenopus laevis (xl) or Pasteurella multocida (pm), and originating or being derived in particular from Pasteurella multocida; and
said one or more recombinant nucleic acids encoding a polypeptide having heparosan synthase activity being under the control of an inducible or repressible promoter that is functional in the recombinant cells of the invention and/or under the control of a promoter selected from the group consisting of pCCW12.Sar, pCCW120.sca, pTDH3-1.Sba and pTDH3.Sar.

### Recombinant Nucleic Acids Encoding a Polypeptide Having UDP-Glucose Dehydrogenase Activity

A recombinant cell according to the invention, and in particular a recombinant yeast according to the invention, comprises one or more recombinant nucleic acids encoding a polypeptide having UDP-Glucose dehydrogenase (UDP-GlcDH or HASB) activity.

A polypeptide having UDP-Glucose dehydrogenase (UDP-GlcDH or HASB) activity according to the invention means a polypeptide that converts the intermediate metabolite Uridine-Diphosphate-Glucose (UDP-Glucose) into UDP-Glucuronate.

In an embodiment, one or more of the recombinant nucleic acids encoding a polypeptide having UDP-Glucose dehydrogenase (UDP-GlcDH or HASB) activity are under the control of an inducible or repressible promoter that is functional in recombinant cells of the invention.

One or more of the recombinant nucleic acids encoding a polypeptide having UDP-Glucose dehydrogenase activity may be under the control of a pCCW12 promoter, and in particular may be under the control of a promoter selected from the group consisting of pCCW12.sk and pCCW12.sba.

The one or more recombinant nucleic acids encoding a polypeptide having UDP-Glucose dehydrogenase activity may originate or be derived from Arabidopsis thaliana, Chlorella virus PBCV1 or Streptococcus zooepidemicus, and in particular originates or is derived from Arabidopsis thaliana or Chlorella virus PBCV1.

A recombinant cell according to the invention, and in particular a recombinant yeast according to the invention, may comprise between 1 and 7, in particular between 2 and 7 recombinant nucleic acids encoding a polypeptide having UDP-Glucose dehydrogenase activity. A recombinant cell according to the invention, and in particular a recombinant yeast according to the invention, may for example comprise 2 or 5 recombinant nucleic acids encoding a polypeptide having UDP-Glucose dehydrogenase activity.

Illustratively, the one or more recombinant nucleic acids encoding a polypeptide having UDP-Glucose dehydrogenase activity may be inserted within the JLP1 gene and/or within the LEU2 gene, and/or within the SAM3 gene of the recombinant cell, and in particular of the recombinant yeast, as it is shown in the examples herein.

In an embodiment of the invention, a recombinant cell, and in particular a recombinant yeast, of the invention comprises:
between 1 and 7, and in particular between 2 and 7 recombinant nucleic acids encoding a polypeptide having UDP-Glucose dehydrogenase activity;
said one or more recombinant nucleic acids encoding a polypeptide having UDP-Glucose dehydrogenase activity originating or being derived from Arabidopsis thaliana, Chlorella virus PBCV1 or Streptococcus zooepidemicus, and originating or being derived in particular from Arabidopsis thaliana or Chlorella virus PBCV1; and
said one or more recombinant nucleic acids encoding a polypeptide having UDP-Glucose dehydrogenase activity being under the control of an inducible or repressible promoter that is functional in the recombinant cells of the invention and/or under the control of a pCCW12 promoter, and in particular may be under the control of a promoter selected from the group consisting of pCCW12.sk and pCCW12.sba.

### Recombinant Nucleic Acids Encoding a Polypeptide Having a Glucuronosyl-Disulfoglucosamine Glucuronidase Activity

A recombinant cell according to the invention, and in particular a recombinant yeast according to the invention, comprises one or more recombinant nucleic acids encoding a polypeptide having glucuronosyl-disulfoglucosamine glucuronidase activity.

A polypeptide having glucuronosyl-disulfoglucosamine glucuronidase activity according to the invention means a polypeptide that degrades heparosan, i.e. that converts a heparosan of a given molecular weight into a heparosan of a lower molecular weight.

As previously indicated, polypeptides having glucuronosyl-disulfoglucosamine glucuronidase activity of the present invention comprise a secretion signal.

In an embodiment, polypeptide having glucuronosyl-disulfoglucosamine glucuronidase activity comprise a secretion signal but no anchoring signal.

In an embodiment, polypeptide having glucuronosyl-disulfoglucosamine glucuronidase activity comprise both a secretion signal and an anchoring signal.

In an embodiment, the said one or more recombinant nucleic acids encoding a polypeptide having glucuronosyl-disulfoglucosamine glucuronidase activity are under the control of an inducible or repressible promoter that is functional in the recombinant cells of the invention.

One or more of the recombinant nucleic acids encoding a polypeptide having glucuronosyl-disulfoglucosamine glucuronidase activity may be under the control of a promoter selected from the group consisting of pTEF1, pCCW12, pCCW12.sba, pCCW12.Sar, pPDC1, pTEF3, pTDH3, pNUP57 and pCCW10.ago.

In a particular embodiment, the nucleic acid encoding a polypeptide having a glucuronosyl-disulfoglucosamine glucuronidase activity is under the control of a promoter selected from the group consisting of pTEF1, pCCW12, pCCW12.sba, pCCW12.Sar, pPDC1, pTEF3, pTDH3, pNUP57 and pCCW10.ago.

The said one or more recombinant nucleic acids encoding a polypeptide having glucuronosyl-disulfoglucosamine glucuronidase activity may originate or be derived from Pedobacter heparinus (Ph), Cupiennius salei (Csa), Loxosceles intermedia (Li), Hirudo nipponia (Hn), Bothrops atrox (Ba) or Tityus serrulatus (Ts) as shown in the examples herein. In a particular embodiment, the said one or more recombinant nucleic acids encoding a polypeptide having glucuronosyl-disulfoglucosamine glucuronidase activity originates or is derived from Pedobacter heparinus (Ph).

A recombinant cell according to the invention, and in particular a recombinant yeast according to the invention, may comprise only one recombinant nucleic acid encoding a polypeptide having glucuronosyl-disulfoglucosamine glucuronidase activity. A recombinant cell according to the invention, and in particular a recombinant yeast according to the invention, may alternatively comprise between 1 and 8, in particular between 1 to 5 recombinant nucleic acid encoding a polypeptide having glucuronosyl-disulfoglucosamine glucuronidase activity.

Illustratively, the one or more recombinant nucleic acids encoding a polypeptide having glucuronosyl-disulfoglucosamine glucuronidase activity may be inserted within the JLP1 gene and/or within the LYPI gene and/or within the SAM3 gene and/or within the HIS3 gene, as it is shown in the examples herein.

### Recombinant Nucleic Acids Encoding a Polypeptide Having Glutamine-Fructose-6-Phosphate Amidotransferase Activity

A recombinant cell according to the invention, and in particular a recombinant yeast cell according to the invention, may comprise one or more recombinant nucleic acids encoding a polypeptide having glutamine-fructose-6-phosphate amidotransferase (GFA1) activity.

A polypeptide having glutamine-fructose-6-phosphate amidotransferase activity according to the invention means a polypeptide that converts fructose-6-phosphate into glucosamine-6-phosphate.

In an embodiment, one or more of the recombinant nucleic acids encoding a polypeptide having glutamine-fructose-6-phosphate amidotransferase activity are under the control of an inducible or repressible promoter that is functional in the recombinant cells of the invention.

One or more of the recombinant nucleic acids encoding a polypeptide having glutamine-fructose-6-phosphate amidotransferase synthase activity may be under the control of a pTEF1 promoter, and in particular pTEF1.Ago.

The said one or more recombinant nucleic acids encoding a polypeptide having glutamine-fructose-6-phosphate amidotransferase activity may originate or be derived from the group consisting of Saccharomyces cerevisiae and Chlorella virus PBCV1.

A recombinant cell according to the invention, and in particular a recombinant yeast according to the invention, may comprise between 1 and 5, and in particular between 1 and 3 recombinant nucleic acid(s) encoding a polypeptide having glutamine-fructose-6-phosphate amidotransferase activity.

Illustratively, the one or more recombinant nucleic acids encoding a polypeptide having glutamine-fructose-6-phosphate amidotransferase activity may be inserted within the SAM3 gene and/or within the JLP1 gene of the recombinant cell, and in particular of the recombinant yeast, as it is shown in the examples herein.

In an embodiment of the invention, a recombinant cell, and in particular a recombinant yeast, of the invention comprises:
between 1 and 5, and in particular between 1 and 3 recombinant nucleic acid(s) encoding a polypeptide having glutamine-fructose-6-phosphate amidotransferase activity;
said recombinant nucleic acid encoding a polypeptide having glutamine-fructose-6-phosphate amidotransferase activity originating or being derived from the group consisting of Saccharomyces cerevisiae and Chlorella virus PBCV1; and
said recombinant nucleic acid encoding a polypeptide having glutamine-fructose-6-phosphate amidotransferase activity being under the control of a pTEF1 promoter, and in particular pTEF1.Ago.

### Recombinant Nucleic Acids Encoding a Polypeptide Having UDP-N-Acetylglucosamine Pyrophosphorylase Activity

A recombinant cell according to the invention, and in particular a recombinant yeast cell according to the invention, may comprise one or more recombinant nucleic acids encoding a polypeptide having UDP-N-acetylglucosamine pyrophosphorylase activity.

A polypeptide having UDP-N-acetylglucosamine pyrophosphorylase activity according to the invention means a polypeptide that converts N-acetyl-glucosamine into UDP-N-acetyl-glucosamine.

In an embodiment, one or more of the recombinant nucleic acids encoding a polypeptide having UDP-N-acetylglucosamine pyrophosphorylase activity are under the control of an inducible or repressible promoter that is functional in the recombinant cells of the invention, such as for example the inducible or repressible promoters pMET6 or pCUP1.

One or more of the recombinant nucleic acids encoding a polypeptide having UDP-N-acetylglucosamine pyrophosphorylase activity may be under the control of the promoter pTDH3.

The said one or more recombinant nucleic acids encoding a polypeptide having UDP-N-acetylglucosamine pyrophosphorylase activity may originate or be derived from Saccharomyces cerevisiae, as shown in the examples herein.

A recombinant cell according to the invention, and in particular a recombinant yeast according to the invention, may comprise between 5 and 15, in particular between 5 and 12 recombinant nucleic acids encoding a polypeptide having UDP-N-acetylglucosamine pyrophosphorylase activity. A recombinant cell according to the invention, and in particular a recombinant yeast according to the invention, may for example comprise 8 or 10 recombinant nucleic acids encoding a polypeptide having UDP-N-acetylglucosamine pyrophosphorylase activity.

Illustratively, the one or more recombinant nucleic acids encoding a polypeptide having UDP-N-acetylglucosamine pyrophosphorylase activity may be inserted within the HIS3 gene and/or within the JLP1 gene and/or within the SAM3 gene of the recombinant cell, and in particular of the recombinant yeast, as it is shown in the examples herein.

In an embodiment of the invention, a recombinant cell, and in particular a recombinant yeast, of the invention comprises:
between 5 and 15, in particular between 5 and 12 recombinant nucleic acids encoding a polypeptide having UDP-N-acetylglucosamine pyrophosphorylase activity;
said one or more recombinant nucleic acids encoding a polypeptide having UDP-N-acetylglucosamine pyrophosphorylase activity originating or being derived from Saccharomyces cerevisiae; and
said one or more recombinant nucleic acids encoding a polypeptide having UDP-N-acetylglucosamine pyrophosphorylase activity being under the control of an inducible or repressible promoter that is functional in the recombinant cells of the invention, such as for example the inducible or repressible promoters pMET6 or pCUP1 and/or under the control of the promoter pTDH3.

### Recombinant Nucleic Acids Encoding a Polypeptide Having Phosphoglucomutase-1 Activity

A recombinant cell according to the invention, and in particular a recombinant yeast according to the invention, may comprise one or more recombinant nucleic acids encoding a polypeptide having Phosphoglucomutase-1 (PGM1) activity.

A polypeptide having Phosphoglucomutase-1 (PGM1) activity according to the invention means a polypeptide that converts Glucose-6-phosphate into the intermediate metabolite Glucose-1-phosphate.

In an embodiment, one or more of the recombinant nucleic acids encoding a polypeptide having Phosphoglucomutase-1 (PGM1) activity are under the control of an inducible or repressible promoter that is functional in recombinant cells of the invention, such as for example the inducible or repressible promoters.

One or more of the recombinant nucleic acids encoding a polypeptide having Phosphoglucomutase-1 (PGM1) activity may be under the control of the promoter pPDC1.

The one or more recombinant nucleic acids encoding a polypeptide having Phosphoglucomutase-1 (PGM1) activity may originate or be derived from Saccharomyces cerevisiae, as shown in the examples herein.

A recombinant cell according to the invention, and in particular a recombinant yeast according to the invention, may comprise only one recombinant nucleic acid encoding a polypeptide having Phosphoglucomutase-1 (PGM1) activity.

Illustratively, the one or more recombinant nucleic acids encoding a polypeptide having Phosphoglucomutase-1 activity may be inserted within the SAM3 gene of the recombinant cell, and in particular of the recombinant yeast, as it is shown in the examples herein.

In an embodiment of the invention, a recombinant cell, and in particular a recombinant yeast, of the invention comprises:
only one recombinant nucleic acid encoding a polypeptide having Phosphoglucomutase-1 activity;
said recombinant nucleic acid encoding a polypeptide having Phosphoglucomutase-1 activity originating or being derived from Saccharomyces cerevisiae; and
said recombinant nucleic acid encoding a polypeptide having Phosphoglucomutase-1 activity being under the control of the promoter pPDC1.

### Recombinant Nucleic Acids Encoding a Polypeptide Having UTP-Glucose-1-Phosphate Uridylyltransferase Activity

A recombinant cell according to the invention, and in particular a recombinant yeast according to the invention, may comprise one or more recombinant nucleic acids encoding a polypeptide having UTP-glucose-1-phosphate uridylyltransferase (UGP1) activity.

A polypeptide having UTP-glucose-1-phosphate uridylyltransferase (UGP1) activity according to the invention means a polypeptide that converts the intermediate metabolite Glucose-1-Phosphate into the intermediate metabolite UDP-glucose.

In an embodiment, one or more of the recombinant nucleic acids encoding a polypeptide having UTP-glucose-1-phosphate uridylyltransferase activity are under the control of an inducible or repressible promoter that is functional in recombinant cells of the invention, such as for example the inducible or repressible promoters pSAM1 or pCUP1.

One or more of the recombinant nucleic acids encoding a polypeptide having UTP-glucose-1-phosphate uridylyltransferase activity may be under the control of a promoter selected from the group consisting of pPDC1 and pENO2.

The one or more recombinant nucleic acids encoding a polypeptide having UTP-glucose-1-phosphate uridylyltransferase activity may originate or be derived from Saccharomyces cerevisiae, as shown in the examples herein.

A recombinant cell according to the invention, and in particular a recombinant yeast according to the invention, may comprise between 5 and 15, in particular between 5 and 12 recombinant nucleic acids encoding a polypeptide having UTP-glucose-1-phosphate uridylyltransferase activity. A recombinant cell according to the invention, and in particular a recombinant yeast according to the invention, may for example comprise 8 or 11 recombinant nucleic acids encoding a polypeptide having UTP-glucose-1-phosphate uridylyltransferase activity.

Illustratively, the one or more recombinant nucleic acids encoding a polypeptide having UTP-glucose-1-phosphate uridylyltransferase activity gene may be inserted within the HIS3 gene and/or within the JLP1 gene and/or within the SAM3 gene of the recombinant cell, and in particular of the recombinant yeast, as it is shown in the examples herein.

In an embodiment of the invention, a recombinant cell, and in particular a recombinant yeast, of the invention comprises:
between 5 and 15, in particular between 5 and 12 recombinant nucleic acids encoding a polypeptide having UTP-glucose-1-phosphate uridylyltransferase activity;
said one or more recombinant nucleic acids encoding a polypeptide having UTP-glucose-1-phosphate uridylyltransferase activity originating or being derived from Saccharomyces cerevisiae; and
said one or more recombinant nucleic acids encoding a polypeptide having UTP-glucose-1-phosphate uridylyltransferase activity being under the control of an inducible or repressible promoter that is functional in the recombinant cells of the invention, such as for example the inducible or repressible promoters pSAM1 or pCUP1 and/or under the control of a promoter selected from the group consisting of pPDC1 and pENO2.

### Recombinant Nucleic Acids Encoding a Polypeptide Having Glucosamine-6-Phosphate N-Acetyltransferase Activity

A recombinant cell according to the invention, and in particular a recombinant yeast cell according to the invention, may comprise one or more recombinant nucleic acids encoding a polypeptide having Glucosamine-6-phosphate N-acetyltransferase (GNA1) activity.

A polypeptide having Glucosamine-6-phosphate N-acetyltransferase (GNA1) activity according to the invention means a polypeptide that converts Glucosamine-6-phosphate into N-acetyl-glucosamine-6-phosphate.

In an embodiment, one or more of the recombinant nucleic acids encoding a polypeptide having Glucosamine-6-phosphate N-acetyltransferase activity are under the control of an inducible or repressible promoter that is functional in the recombinant cells of the invention.

One or more of the recombinant nucleic acids encoding a polypeptide having Glucosamine-6-phosphate N-acetyltransferase activity may be under the control of the promoter pCWP2.

The said one or more recombinant nucleic acids encoding a polypeptide having Glucosamine-6-phosphate N-acetyltransferase activity may originate or be derived from Saccharomyces cerevisiae, as shown in the examples herein.

A recombinant cell according to the invention, and in particular a recombinant yeast according to the invention, may comprise only one recombinant nucleic acid encoding a polypeptide having Glucosamine-6-phosphate N-acetyltransferase activity.

Illustratively, the one or more recombinant nucleic acids encoding a polypeptide having Glucosamine-6-phosphate N-acetyltransferase activity may be inserted within the SAM3 gene of the recombinant cell, and in particular of the recombinant yeast, as it is shown in the examples herein.

In an embodiment of the invention, a recombinant cell, and in particular a recombinant yeast, of the invention comprises:
only one recombinant nucleic acid encoding a polypeptide having Glucosamine-6-phosphate N-acetyltransferase activity;
said recombinant nucleic acid encoding a polypeptide having Glucosamine-6-phosphate N-acetyltransferase activity originating or being derived from Saccharomyces cerevisiae; and
said recombinant nucleic acid encoding a polypeptide having Glucosamine-6-phosphate N-acetyltransferase activity being under the control of the promoter pCWP2.

### Recombinant Nucleic Acids Encoding a Polypeptide Having Phosphoacetylglucosamine Mutase Activity

A recombinant cell according to the invention, and in particular a recombinant yeast cell according to the invention, may comprise one or more recombinant nucleic acids encoding a polypeptide having phosphoacetylglucosamine mutase (PCM1) activity.

A polypeptide having phosphoacetylglucosamine mutase (PCM1) activity according to the invention means a polypeptide that converts N-acetyl-glucosamine-6-phosphate N-acetyl-glucosamine-1-phosphate.

In an embodiment, one or more of the recombinant nucleic acids encoding a polypeptide having phosphoacetylglucosamine mutase activity are under the control of an inducible or repressible promoter that is functional in the recombinant cells of the invention.

One or more of the recombinant nucleic acids encoding a polypeptide having phosphoacetylglucosamine mutase activity may be under the control of a pTEF1 promoter.

The said one or more recombinant nucleic acids encoding a polypeptide having phosphoacetylglucosamine mutase activity can originate or be derived from Saccharomyces cerevisiae, as shown in the examples herein.

A recombinant cell according to the invention, and in particular a recombinant yeast according to the invention, can comprise only one recombinant nucleic acid encoding a polypeptide having phosphoacetylglucosamine mutase activity.

Illustratively, the one or more recombinant nucleic acids encoding a polypeptide having phosphoacetylglucosamine mutase activity may be inserted within the SAM3 gene of the recombinant cell, and in particular of the recombinant yeast, as it is shown in the examples herein.

In an embodiment of the invention, a recombinant cell, and in particular a recombinant yeast, of the invention comprises:
only one recombinant nucleic acid encoding a polypeptide having phosphoacetylglucosamine mutase activity;
said recombinant nucleic acid encoding a polypeptide having phosphoacetylglucosamine mutase activity originating or being derived from Saccharomyces cerevisiae; and
said recombinant nucleic acid encoding a polypeptide having phosphoacetylglucosamine mutase activity being under the control of a pTEF1 promoter.

### Heparosan Synthase (HSS)

The heparosan synthase enzyme is a protein which is described in the art for catalyzing the conversion of UDP-glucuronate or UDP-N-acetyl-glucose into heparosan. The heparosan synthase originating from Streptococcus zooepidemicus, Chlorella virus PBCV1, Xenopus laevis or Pasteurella multocida may be termed HSS.

A method implemented to measure the activity level of a polypeptide having heparosan synthase activity belongs to the general knowledge of the one skilled in the art.

In this regard, the one skilled in the art may advantageously refer to the method of colorimetric determination after treatment with concentrated sulfuric acid and carbazole described by Bitter and Muir (Analytical Biochemistry, 4, 330-334, 1962).

Preferred polypeptide having heparosan synthase activity in the present specification are enzymes having a nucleic acid sequence selected from the group consisting of SEQ ID NO: 1 (HSS1) and SEQ ID NO: 2 (HSS2).

According to a preferred embodiment, the one or more recombinant nucleic acids encoding a polypeptide having heparosan synthase activity may originate or be derived from organisms preferably selected in a group consisting of prokaryotic organisms and eukaryotic organisms. In some embodiments, the one or more recombinant nucleic acids encoding a polypeptide having heparosan synthase activity may originate or be derived from archaebacteria. In some embodiments, the one or more recombinant nucleic acids encoding a polypeptide having heparosan synthase activity may originate or be derived from bacteria, and especially from Streptococcus zooepidemicus (Sz), Chlorella virus PBCV1 (Vir), Xenopus laevis (X1) or Pasteurella multocida (Pm), and in particular from Pasteurella multocida.

According to a yet preferred embodiment, the one or more recombinant nucleic acids encoding a polypeptide having heparosan synthase activity may be selected from the group consisting of nucleic acid sequences having (i) at least 65%, advantageously at least 70%, preferably at least 80%, nucleic acid identity with a nucleic acid sequence as set forth as sequence SEQ ID NO: 1 (HSS1-Pm) or SEQ ID NO: 2 (HSS2-Pm), and (ii) a biological activity of the same nature as the nucleic acid sequence having, respectively, the nucleic acid sequence as set forth as sequence SEQ ID NO: 1 (HSS1-Pm) or SEQ ID NO: 2 (HSS2-Pm).

A biological activity of the same nature regarding this sequence is, as previously explained, the capacity to code for an enzyme that converts UDP-glucuronate or UDP-N-acetyl-glucose into heparosan.

As described herein, a nucleic acid sequence having at least 65% nucleotide identity with a reference nucleic acid sequence encompasses nucleic acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

As described herein, a nucleic acid sequence having at least 70% nucleotide identity with a reference nucleic acid sequence encompasses nucleic acid sequences having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

For the amino acid sequence of the polypeptide having heparosan synthase activity originating from Pasteurella multocida, the one skilled in the art may refer, respectively, to the accession numbers Q8RP78 and Q5SGE1 in the UniProt database, or to SEQ ID NO: 3 (HSS1-Pm) or SEQ ID NO: 4 (HSS2-Pm) described herein.

According to another particular embodiment, the one or more recombinant nucleic acids encoding a polypeptide having heparosan synthase activity may be nucleic acid(s) encoding polypeptide having an amino acid sequence selected from the group consisting of sequences having at least 50%, advantageously at least 65%, preferably at least 80%, amino acid identity with the amino acid sequence set forth as SEQ ID NO: 3 (Pm HSS1) or SEQ ID NO: 4 (Pm HSS2), and also a biological activity of the same nature as the amino acid sequence set forth as SEQ ID NO: 3 (Pm HSS1) or SEQ ID NO: 4 (Pm HSS2).

A biological activity of the same nature regarding this sequence is as described previously, i.e. the capacity to catalyze the conversion of UDP-glucuronate or UDP-N-acetyl-glucose into heparosan.

As described herein, an amino acid sequence having at least 50% amino acid identity with a reference amino acid sequence encompasses amino acid sequences having at least 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 65% amino acid identity with a reference amino acid sequence encompasses amino acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the one or more recombinant nucleic acids encoding a polypeptide having heparosan synthase activity in the present invention is regulated by at least one promoter and at least one terminator, such as herein after defined more in details, which are present in 5′ and 3′ position respectively of the one or more recombinant nucleic acids encoding a polypeptide having heparosan synthase activity.

### UDP-Glucose Dehydrogenase (UDP-GlcDH OR HASB)

The UDP-Glucose dehydrogenase is a protein which is known in the art to catalyze the conversion of UDP-glucose into UDP-glucuronate. The UDP-Glucose dehydrogenase originating from the genome of Arabidopsis thaliana, Chlorella virus PBCV1 or Streptococcus zooepidemicus may be termed HASB.

A method implemented to measure the activity level of a polypeptide having UDP-Glucose dehydrogenase activity belongs to the general knowledge of the one skilled in the art.

In this regard, the one skilled in the art may advantageously refer to the method described by Oka and Jigami (FEBS Journal 273, 2645-2657, 2006).

Preferred polypeptide having UDP-Glucose dehydrogenase activity in the present specification is an enzyme having an EC number 1.1.1.22.

According to a preferred embodiment, the one or more recombinant nucleic acids encoding a polypeptide having UDP-Glucose dehydrogenase activity may originate or be derived from organisms preferably selected in a group comprising prokaryotic organisms and eukaryotic organisms. In some embodiments, the one or more recombinant nucleic acids encoding a polypeptide having UDP-Glucose dehydrogenase activity may originate or be derived from archaebacteria. In some preferred embodiments, the one or more recombinant nucleic acids encoding a polypeptide having UDP-Glucose dehydrogenase activity may originate or be derived from yeast, and especially from Arabidopsis thaliana, Chlorella virus PBCV1 or Streptococcus zooepidemicus, and more particularly from Arabidopsis thaliana or Chlorella virus PBCV1.

According to a yet preferred embodiment, the one or more recombinant nucleic acids encoding a polypeptide having UDP-Glucose dehydrogenase activity may be selected from the group consisting of nucleic acid sequences having (i) at least 65%, advantageously at least 70%, preferably at least 80%, nucleic acid identity with a nucleic acid as set forth as sequence SEQ ID NO: 5 (At), SEQ ID NO. 6 (Vir) or SEQ ID NO. 7 (Vir), and (ii) a biological activity of the same nature as the nucleic acid as set forth as sequence SEQ ID NO: 5 (At), SEQ ID NO. 6 (Vir) or SEQ ID NO. 7 (Vir). The nucleic acids set forth as sequences SEQ ID NO: 5 (At), SEQ ID NO: 6 (Vir) and SEQ ID NO. 7 (Vir) encode a polypeptide having UDP-Glucose dehydrogenase activity originating, respectively, from Arabidopsis thaliana (At) or Chlorella virus PBCV1 (Vir) that may herein also be collectively termed HASB.

A biological activity of the same nature regarding this sequence is, as previously explained, the capacity to code for a polypeptide that converts UDP-glucose into UDP-glucuronate.

As described herein, a nucleic acid sequence having at least 65% nucleotide identity with a reference nucleic acid sequence encompasses nucleic acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

As described herein, a nucleic acid sequence having at least 70% nucleotide identity with a reference nucleic acid sequence encompasses nucleic acid sequences having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequences, and also a biological activity of the same nature as the said reference nucleic acid sequences.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequences.

For the amino acid sequence of the polypeptide having UDP-Glucose dehydrogenase activity from Arabidopsis thaliana, Chlorella virus PBCV1 or Streptococcus zooepidemicus, the one skilled in the art may refer to the accession numbers NP_173979.1, NP_048965 or KIS19289, respectively, in the UniProt database, or to the sequences as set forth in SEQ ID NO: 8 (At) and SEQ ID NO. 9 (Vir) described herein.

According to another particular embodiment, the one or more recombinant nucleic acids encoding a polypeptide having UDP-Glucose dehydrogenase activity may be nucleic acid(s) encoding a polypeptide having an amino acid sequence selected from the group consisting of sequences having at least 55%, advantageously at least 65%, preferably at least 80%, amino acid identity with the amino acid sequence of SEQ ID NO: 8 (At) and SEQ ID NO. 9 (Vir), and also a biological activity of the same nature as the amino acid sequence of SEQ ID NO: 8 (At) and SEQ ID NO. 9 (Vir).

A biological activity of the same nature regarding this sequence is as described previously, i.e. the capacity to catalyze the conversion of UDP-glucose into UDP-glucuronate.

As described herein, an amino acid sequence having at least 55% amino acid identity with a reference amino acid sequence encompasses amino acid sequences having at least 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 65% amino acid identity with a reference amino acid sequence encompasses amino acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the one or more recombinant nucleic acids encoding a polypeptide having UDP-Glucose dehydrogenase activity in the present invention is regulated by at least one promoter and at least one terminator, such as herein after defined more in details, which are present in 5′ and 3′ position respectively of the one or more recombinant nucleic acids encoding a polypeptide having UDP-Glucose dehydrogenase activity.

### Glucuronosyl-Disulfoglucosamine Glucuronidase

The glucuronosyl-disulfoglucosamine glucuronidase enzyme is a protein which is described in the art for catalyzing the degradation of heparosan molecules into smaller heparosan molecules. The glucuronosyl-disulfoglucosamine glucuronidase encoded by the genome of Pedobacter heparinus may be termed HEPC.

The polypeptide having glucuronosyl-disulfoglucosamine glucuronidase activity of the invention may possess both a secretion signal and an anchoring signal or a secretion signal and no anchoring signal or a secretion-anchor signal with a dual secretion and anchoring function.

A method implemented to measure the activity level of a polypeptide having glucuronosyl-disulfoglucosamine glucuronidase activity belongs to the general knowledge of the one skilled in the art.

In this regard, the one skilled in the art may advantageously monitor the molecular weight of the obtained heparosan on an agarose gel.

Preferred polypeptide having glucuronosyl-disulfoglucosamine glucuronidase activity in the present specification is an enzyme having an EC number of 3.2.1.56.

According to a preferred embodiment, the one or more recombinant nucleic acids encoding a polypeptide having glucuronosyl-disulfoglucosamine glucuronidase activity may originate or be derived from organisms preferably selected in a group comprising prokaryotic organisms and eukaryotic organisms. In some embodiments, the one or more recombinant nucleic acids encoding a polypeptide having glucuronosyl-disulfoglucosamine glucuronidase activity may originate or be derived from archaebacteria. In some embodiments, the one or more recombinant nucleic acids encoding a polypeptide having glucuronosyl-disulfoglucosamine glucuronidase activity may originate or be derived from organisms preferably selected from yeasts. In some other preferred embodiments, the one or more recombinant nucleic acids encoding a polypeptide having glucuronosyl-disulfoglucosamine glucuronidase activity may originate or be derived from Pedobacter heparinus.

According to a yet preferred embodiment, the one or more recombinant nucleic acids encoding a polypeptide having glucuronosyl-disulfoglucosamine glucuronidase activity may be selected from the group consisting of nucleic acid sequences having at least 65%, advantageously at least 70%, preferably at least 80%, nucleic acid identity with a nucleic acid of SEQ ID NO: 10 (Ph) or of SEQ ID NO: 12 (Ph) and also a biological activity of the same nature as the nucleic acid of SEQ ID NO: 10 (Ph) or of SEQ ID NO: 12 (Ph). The nucleic acids of SEQ ID NO: 10 (Ph) and of SEQ ID NO: 12 (Ph) encode a polypeptide having glucuronosyl-disulfoglucosamine glucuronidase activity, comprises a secretion signal and no anchoring signal, and originate or derive from Pedobacter heparinus.

According to another preferred embodiment, the one or more recombinant nucleic acids encoding a polypeptide having glucuronosyl-disulfoglucosamine glucuronidase activity may be selected from the group consisting of nucleic acid sequences having at least 65%, advantageously at least 70%, preferably at least 80%, nucleic acid identity with a nucleic acid of SEQ ID NO: 11 (Ph) or of SEQ ID NO: 13 (Ph) and also a biological activity of the same nature as the nucleic acid of SEQ ID NO: 11 (Ph) or of SEQ ID NO: 13 (Ph). The nucleic acids of SEQ ID NO: 11 (Ph) and of SEQ ID NO: 13 (Ph) encode a polypeptide having glucuronosyl-disulfoglucosamine glucuronidase activity, comprises a secretion signal and an anchoring signal, and originate or derive from Pedobacter heparinus.

A biological activity of the same nature regarding this sequence is, as previously explained, the capacity to code for a polypeptide that catalyzes the degradation of heparosan molecules into smaller heparosan molecules.

As described herein, a nucleic acid sequence having at least 65% nucleotide identity with a reference nucleic acid sequence encompasses nucleic acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

As described herein, a nucleic acid sequence having at least 70% nucleotide identity with a reference nucleic acid sequence encompasses nucleic acid sequences having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

For the amino acid sequence of the one or more recombinant nucleic acids encoding a polypeptide having glucuronosyl-disulfoglucosamine glucuronidase activity from Pedobacter heparinus, the one skilled in the art may refer to the accession numbers Q59289 in the UniProt database, corresponding to both SEQ ID NO: 10 (Ph) and SEQ ID NO: 12 (Ph) described herein.

According to another particular embodiment, the one or more recombinant nucleic acids encoding a polypeptide having glucuronosyl-disulfoglucosamine glucuronidase activity may be nucleic acid(s) encoding polypeptide having an amino acid sequence selected from the group consisting of sequences having at least 50%, advantageously at least 65%, preferably at least 80%, amino acid identity with the amino acid sequence of SEQ ID NO: 14 (Ph) which comprise a secretion signal and no anchoring signal, and also a biological activity of the same nature as the amino acid sequence SEQ ID NO: 14 (Ph).

According to another particular embodiment, the one or more recombinant nucleic acids encoding a polypeptide having glucuronosyl-disulfoglucosamine glucuronidase activity may be nucleic acid(s) encoding polypeptide having an amino acid sequence selected from the group consisting of sequences having at least 55%, advantageously at least 65%, preferably at least 80%, amino acid identity with the amino acid sequence of SEQ ID NO: 15 (Ph) which comprise a secretion signal and an anchoring signal, and also a biological activity of the same nature as the amino acid sequence of SEQ ID NO: 15 (Ph).

A biological activity of the same nature regarding this sequence is as described previously, i.e. the capacity to catalyze the degradation of heparosan molecules into smaller heparosan molecules.

As described herein, an amino acid sequence having at least 55% amino acid identity with a reference amino acid sequence encompasses amino acid sequences having at least 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 65% amino acid identity with a reference amino acid sequence encompasses amino acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the one or more recombinant nucleic acids encoding a polypeptide having glucuronosyl-disulfoglucosamine glucuronidase activity in the present invention is regulated by at least one promoter and at least one terminator, such as herein after defined more in details, which are present in 5′ and 3′ position respectively of the one or more recombinant nucleic acids encoding a polypeptide having glucuronosyl-disulfoglucosamine glucuronidase activity.

### Glutamine-Fructose-6-Phosphate Amidotransferase (GFA1)

The glutamine-fructose-6-phosphate amidotransferase enzyme is a protein which is described in the art for catalyzing the conversion of fructose-6-phosphate into glucosamine-6-phosphate. The glutamine-fructose-6-phosphate amidotransferase originating from Saccharomyces cerevisiae and Chlorella virus PBCV1 may be termed GFA1.

A method implemented to measure the activity level of a polypeptide having glutamine-fructose-6-phosphate amidotransferase activity belongs to the general knowledge of the one skilled in the art.

In this regard, the one skilled in the art may advantageously refer to the method described by Shiga Shibatan and Hiroaki Kitazawa (Plant Biotechnology 26, 149-152, 2009).

Preferred polypeptide having glutamine-fructose-6-phosphate amidotransferase activity in the present invention is an enzyme having an EC number of n° EC 2.6.1.16.

According to a preferred embodiment, the one or more recombinant nucleic acids encoding a polypeptide having glutamine-fructose-6-phosphate amidotransferase activity may originate or be derived from organisms preferably selected in a group comprising prokaryotic organisms and eukaryotic organisms. In some embodiments, the one or more recombinant nucleic acids encoding a polypeptide having glutamine-fructose-6-phosphate amidotransferase activity may originate or be derived from archaebacteria. In some embodiments, the one or more recombinant nucleic acids encoding a polypeptide having glutamine-fructose-6-phosphate amidotransferase activity may originate or be derived from organisms preferably selected from Bacillus subtilis, and yeasts. In some other preferred embodiments, the one or more recombinant nucleic acids encoding a polypeptide having glutamine-fructose-6-phosphate amidotransferase activity may originate or be derived from Saccharomyces cerevisiae or from Chlorella virus PBCV1.

According to a yet preferred embodiment, the one or more recombinant nucleic acids encoding a polypeptide having glutamine-fructose-6-phosphate amidotransferase activity may be selected from the group consisting of nucleic acid sequences having (i) at least 65%, advantageously at least 70%, preferably at least 80%, nucleic acid identity with the nucleic acid sequence as set forth as sequence SEQ ID NO: 16 (Sc) or EQ, and (ii) a biological activity of the same nature as the nucleic acid sequence as set forth as sequence SEQ ID NO: 16 (Sc). The nucleic acid as set forth as sequence SEQ ID NO: 16 encodes a polypeptide having glutamine-fructose-6-phosphate amidotransferase activity originating from Saccharomyces cerevisiae, that may also be termed GFA1.

According to yet another embodiment, the one or more recombinant nucleic acids encoding a polypeptide having glutamine-fructose-6-phosphate amidotransferase activity may be selected from the group consisting of nucleic acid sequences having (i) at least 65%, advantageously at least 70%, preferably at least 80%, nucleic acid identity with the nucleic acid sequence as set forth as sequence SEQ ID NO: 15 or SEQ ID NO: 16, and (ii) a biological activity of the same nature as the nucleic acid sequence as set forth as sequence SEQ ID NO: 17 or SEQ ID NO: 18. The nucleic acid sequence as set forth as sequence SEQ ID NO: 17 or SEQ ID NO: 18 encodes a polypeptide having glutamine-fructose-6-phosphate amidotransferase activity originating from Chlorella virus PBCV1.

A biological activity of the same nature regarding this sequence is, as previously explained, the capacity to code for a polypeptide that converts fructose-6-phosphate into glucosamine-6-phosphate.

As described herein, a nucleic acid sequence having at least 65% nucleotide identity with a reference nucleic acid sequence encompasses nucleic acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

As described herein, a nucleic acid sequence having at least 70% nucleotide identity with a reference nucleic acid sequence encompasses nucleic acid sequences having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

For the amino acid sequence of the polypeptide having glutamine-fructose-6-phosphate amidotransferase activity originating from Saccharomyces cerevisiae, the one skilled in the art may refer to the accession number NP8012818 in the UniProt database, or to the sequence SEQ ID NO: 19 described herein.

For the amino acid sequence of the polypeptide having glutamine-fructose-6-phosphate amidotransferase activity originating from Chlorella virus PBCV1, the one skilled in the art may also refer to the accession number NP_048448 in the UniProt database, or to the sequence SEQ ID NO: 20 described herein.

According to another particular embodiment, the one or more recombinant nucleic acids encoding a polypeptide having glutamine-fructose-6-phosphate amidotransferase activity may be nucleic acid(s) encoding polypeptide having an amino acid sequence selected from the group consisting of sequences having at least 35%, advantageously at least 65%, preferably at least 80%, amino acid identity with the amino acid sequence of SEQ ID NO: 19 or with SEQ ID NO: 20, and also a biological activity of the same nature as the amino acid sequence of SEQ ID NO: 19 or with SEQ ID NO: 20.

A biological activity of the same nature regarding this sequence is as described previously, i.e. the capacity to catalyze the conversion of fructose-6-phosphate into glucosamine-6-phosphate.

As described herein, an amino acid sequence having at least 35% amino acid identity with a reference amino acid sequence encompasses amino acid sequences having at least 36%, 37%, 38%, 39%, 40% 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 65% amino acid identity with a reference amino acid sequence encompasses amino acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the polypeptide having glutamine-fructose-6-phosphate amidotransferase activity in the present invention is regulated by at least one promoter and at least one terminator, such as herein after defined more in details, which are present in 5′ and 3′ position respectively of one or more recombinant nucleic acids encoding a polypeptide having glutamine-fructose-6-phosphate amidotransferase activity.

### UDP-N-Acetylglucosamine Pyrophosphorylase (QRI1)

The UDP-N-acetylglucosamine pyrophosphorylase enzyme is a protein which is described in the art for catalyzing the conversion of N-acetyl-glucosamine-6-phosphate into UDP-N-acetyl-glucose. The UDP-N-acetylglucosamine pyrophosphorylase originating from Saccharomyces cerevisiae may be termed QRI1.

A method implemented to measure the activity level of a polypeptide having UDP-N-acetylglucosamine pyrophosphorylase activity belongs to the general knowledge of the one skilled in the art.

In this regard, the one skilled in the art may advantageously refer to the method described by Mio et al. (The Journal of Biological Chemistry, Col. 273, No 23, Jun. 5, 1998, 14392-14397) except that UDP-N-acetyl-glucosamine is detected by LC MS/MS using a Synergi RP Fusion column.

Preferred polypeptide having UDP-N-acetylglucosamine pyrophosphorylase activity in the present invention is an enzyme having an EC number of n° EC 2.7.7.23

According to a preferred embodiment, the one or more recombinant nucleic acids encoding a polypeptide having UDP-N-acetylglucosamine pyrophosphorylase activity may originate or be derived from organisms preferably selected in a group consisting of prokaryotic organisms and eukaryotic organisms. In some embodiments, the one or more recombinant nucleic acids encoding a polypeptide having UDP-N-acetylglucosamine pyrophosphorylase activity may originate or be derived from archaebacteria. In some embodiments, the one or more recombinant nucleic acids encoding a polypeptide having UDP-N-acetylglucosamine pyrophosphorylase activity may originate or be derived from organisms preferably selected from Bacillus subtilis, and yeasts. In some other preferred embodiments, the one or more recombinant nucleic acids encoding a polypeptide having UDP-N-acetylglucosamine pyrophosphorylase activity may originate or be derived from yeasts, and especially from Saccharomyces cerevisiae.

According to a yet preferred embodiment, the one or more recombinant nucleic acids encoding a polypeptide having UDP-N-acetylglucosamine pyrophosphorylase activity may be selected from the group consisting of nucleic acid sequences having (i) at least 65%, advantageously at least 70%, preferably at least 80%, nucleic acid identity with a nucleic acid sequence as set forth as SEQ ID NO: 21, and (ii) a biological activity of the same nature as the nucleic acid sequence as set forth as SEQ ID NO: 21. The nucleic acid sequence set forth as SEQ ID NO: 21 encodes a polypeptide having UDP-N-acetylglucosamine pyrophosphorylase activity originating from Saccharomyces cerevisiae, that may also be termed QRI1.

A biological activity of the same nature regarding this sequence is, as previously explained, the capacity to code for a polypeptide that converts N-acetyl-glucosmine-6-phosphate into UDP-N-acetyl-glucose.

As described herein, a nucleic acid sequence having at least 65% nucleotide identity with a reference nucleic acid sequence encompasses nucleic acid sequences having at least 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

As described herein, a nucleic acid sequence having at least 70% nucleotide identity with a reference nucleic acid sequence encompasses nucleic acid sequences having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

For the amino acid sequence of the polypeptide having UDP-N-acetylglucosamine pyrophosphorylase activity originating from Saccharomyces cerevisiae, the one skilled in the art may refer to the accession number NP_010180 in the UniProt database, or to SEQ ID NO. 22 described herein.

According to another particular embodiment, the one or more recombinant nucleic acids encoding a polypeptide having UDP-N-acetylglucosamine pyrophosphorylase activity may be nucleic acid(s) encoding polypeptide having an amino acid sequence selected from the group consisting of sequences having at least 35%, advantageously at least 45%, preferably at least 80%, amino acid identity with the amino acid sequence set forth as sequence SEQ ID NO. 22, and also a biological activity of the same nature as the amino acid sequence set forth as sequence SEQ ID NO. 22.

A biological activity of the same nature regarding this sequence is as described previously, i.e. the capacity to catalyze the conversion of N-acetyl-glucosmine-6-phosphate into UDP-N-acetyl-glucose.

As described herein, an amino acid sequence having at least 35% amino acid identity with a reference amino acid sequence encompasses amino acid sequences having at least 36%, 37%, 38%, 39%, 40% 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 45% amino acid identity with a reference amino acid sequence encompasses amino acid sequences having at least 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the one or more recombinant nucleic acids encoding a polypeptide having UDP-N-acetylglucosamine pyrophosphorylase activity in the present invention is regulated by at least one promoter and at least one terminator, such as herein after defined more in details, which are present in 5′ and 3′ position respectively of the one or more recombinant nucleic acids encoding a polypeptide having UDP-N-acetylglucosamine pyrophosphorylase activity.

### Phosphoglucomutase-1 (PGM1)

The Phosphoglucomutase-1 enzyme is a protein which is described in the art for catalyzing the conversion of Glucose-6-phosphate into Glucose-1-phosphate. The Phosphoglucomutase-1 originating from Saccharomyces cerevisiae may be termed PGM1.

A method implemented to measure the activity level of a polypeptide facing Phosphoglucomutase-1 activity belongs to the general knowledge of the one skilled in the art.

In this regard, the one skilled in the art may advantageously refer to the method described by Tiwari and Bhat (Biochemical and Biophysical Research Communications 366, 340-345, 2008).

Preferred polypeptide having Phosphoglucomutase-1 activity in the present invention is an enzyme having an EC number of n° 5.4.2.2.

According to a preferred embodiment, the one or more recombinant nucleic acids encoding a polypeptide having Phosphoglucomutase-1 activity may originate from organisms preferably selected in a group consisting of prokaryotic organisms and eukaryotic organisms. In some embodiments, the one or more recombinant nucleic acids encoding a polypeptide having Phosphoglucomutase-1 activity may originate or be derived from archaebacteria. In some embodiments, the one or more recombinant nucleic acids encoding a polypeptide having Phosphoglucomutase-1 activity may originate or be derived from organisms preferably selected from bacteria. In a preferred embodiment, the one or more recombinant nucleic acids encoding a polypeptide having Phosphoglucomutase-1 activity may orginate or be derived from Saccharomyces cerevisiae.

According to an embodiment, the one or more recombinant nucleic acids encoding a polypeptide having Phosphoglucomutase-1 activity may be selected from the group consisting of nucleic acid sequences having (i) at least 80% nucleic acid identity with the nucleic acid sequence set forth as sequence SEQ ID NO: 23, which originates from Saccharomyces cerevisiae, and (ii) a biological activity of the same nature as the nucleic acid sequence set forth as sequence SEQ ID NO: 23.

A biological activity of the same nature regarding this sequence is, as previously explained, the capacity to code for a polypeptide that converts Glucose-6-phosphate into Glucose-1-phosphate.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

For the amino acid sequence of the peptide having Phosphoglucomutase-1 activity from Saccharomyces cerevisiae, the one skilled in the art may refer to the accession number NP33401 in the UniProt database, or to SEQ ID NO: 24 described herein.

According to another particular embodiment, the one or more recombinant nucleic acids encoding a polypeptide having Phosphoglucomutase-1 activity may be nucleic acid(s) encoding polypeptide having an amino acid sequence selected from the group consisting of sequences having at least 80% amino acid identity with the amino acid sequence set forth as sequence SEQ ID NO: 24, and also a biological activity of the same nature as the amino acid sequence set forth as sequence SEQ ID NO: 24.

A biological activity of the same nature regarding this sequence is as described previously, i.e. the capacity to catalyze the conversion of Glucose-6-phosphate into Glucose-1-phosphate.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the one or more recombinant nucleic acids encoding a polypeptide having Phosphoglucomutase-1 activity in the present invention is regulated by at least one promoter and at least one terminator, such as herein after defined more in details, which are present in 5′ and 3′ position respectively of the one or more recombinant nucleic acids encoding a polypeptide having Phosphoglucomutase-1 activity.

### UTP-Glucose-1-Phosphate Uridylyltransferase (UGP1)

The UTP-glucose-1-phosphate uridylyltransferase enzyme is a protein which is described in the art for catalyzing the conversion of Glucose-1-Phosphate into UDP-glucose.

The UTP-glucose-1-phosphate uridylyltransferase originating from Saccharomyces cerevisiae may be termed UGP1.

A method implemented to measure the activity level of a polypeptide having UTP-glucose-1-phosphate uridylyltransferase activity belongs to the general knowledge of the one skilled in the art.

In this regard, the one skilled in the art may advantageously refer to the method described by Roeben (J. Mol. Biol 364, 551-560, 2006).

Preferred polypeptide having UTP-glucose-1-phosphate uridylyltransferase activity in the present invention is an enzyme having an EC number of nº 2.7.7.9.

According to a preferred embodiment, the one or more recombinant nucleic acids encoding a polypeptide having UTP-glucose-1-phosphate uridylyltransferase activity may originate or be derived from organisms preferably selected in a group consisting of prokaryotic organisms and eukaryotic organisms. In some embodiments, the one or more recombinant nucleic acids encoding a polypeptide having UTP-glucose-1-phosphate uridylyltransferase activity may originate or be derived from archaebacteria. In some embodiments, the one or more recombinant nucleic acids encoding a polypeptide having UTP-glucose-1-phosphate uridylyltransferase activity may originate or be derived from organisms preferably selected from bacteria. In a preferred embodiment, the one or more recombinant nucleic acids encoding a polypeptide having UTP-glucose-1-phosphate uridylyltransferase activity may originate or be derived from Saccharomyces cerevisiae.

According to a particular embodiment, the one or more recombinant nucleic acids encoding a polypeptide having UTP-glucose-1-phosphate uridylyltransferase activity may be selected from the group consisting of nucleic acid sequences having (i) at least 80% nucleic acid identity with a nucleic acid as set forth as sequence SEQ ID NO: 25, originating from Saccharomyces cerevisiae, and (ii) a biological activity of the same nature as the nucleic acid as set forth as sequence SEQ ID NO: 25.

A biological activity of the same nature regarding this sequence is, as previously explained, the capacity to code for a polypeptide that converts Glucose-1-phosphate into UDP-glucose.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

For the amino acid sequence of the polypeptide having UTP-glucose-1-phosphate uridylyltransferase activity originating from Saccharomyces cerevisiae, the one skilled in the art may refer to the accession number NP_32861 in the UniProt database, or to the sequence set forth as sequence SEQ ID NO: 26 described herein.

According to another particular embodiment, the one or more recombinant nucleic acids encoding a polypeptide having UTP-glucose-1-phosphate uridylyltransferase activity may be nucleic acid(s) encoding polypeptide having an amino acid sequence selected from the group consisting of sequences having at least 80% amino acid identity with the amino acid sequence set forth as SEQ ID NO: 26, and also a biological activity of the same nature as the amino acid sequence set forth as SEQ ID NO: 26.

A biological activity of the same nature regarding this sequence is as described previously, i.e. the capacity to catalyze the conversion of Glucose-1-phosphate into UDP-glucose.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the one or more recombinant nucleic acids encoding a polypeptide having UTP-glucose-1-phosphate uridylyltransferase activity in the present invention is regulated by at least one promoter and at least one terminator, such as herein after defined more in details, which are present in 5′ and 3′ position respectively of the one or more recombinant nucleic acids encoding a polypeptide having UTP-glucose-1-phosphate uridylyltransferase activity.

### Glucosamine-6-Phosphate N-Acetyltransferase (GNA1)

The Glucosamine-6-phosphate N-acetyltransferase enzyme is a protein which is described in the art for catalyzing the conversion of Glucosamine-6-phosphate into N-acetyl-glucosamine-6-phosphate. The Glucosamine-6-phosphate N-acetyltransferase originating from Saccharomyces cerevisiae may be termed GNA1.

A method implemented to measure the activity level of a polypeptide having Glucosamine-6-phosphate N-acetyltransferase activity belongs to the general knowledge of the one skilled in the art.

In this regard, the one skilled in the art may advantageously refer to the method described by Li et al. (Anal. Biochem. 370, 142-146, 2007).

Preferred polypeptide having Glucosamine-6-phosphate N-acetyltransferase activity in the present invention is an enzyme having an EC number of n° 2.3.1.4.

According to a preferred embodiment, the one or more recombinant nucleic acids encoding a polypeptide having Glucosamine-6-phosphate N-acetyltransferase activity may originate or be derived from organisms preferably selected in a group consisting of prokaryotic organisms and eukaryotic organisms. In some preferred embodiments, the one or more recombinant nucleic acids encoding a polypeptide having Glucosamine-6-phosphate N-acetyltransferase activity may originate or be derived from a yeast, and especially from Saccharomyces cerevisiae.

According to a particular embodiment, the one or more recombinant nucleic acids encoding a polypeptide having Glucosamine-6-phosphate N-acetyltransferase activity may be selected from the group consisting of nucleic acid sequences having (i) at least 80% nucleic acid identity with a nucleic acid sequence as set forth as sequence SEQ ID NO: 27, and (ii) a biological activity of the same nature as the nucleic acid sequence as set forth as sequence SEQ ID NO: 27. The nucleic acid sequence as set forth as sequence SEQ ID NO: 27 encodes a polypeptide having Glucosamine-6-phosphate N-acetyltransferase activity originating from Saccharomyces cerevisiae, that may also be termed GNA1.

A biological activity of the same nature regarding this sequence is, as previously explained, the capacity to code for a polypeptide that converts Glucosamine-6-phosphate into N-acetyl-glucosamine-6-phosphate.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

For the amino acid sequence of the polypeptide having Glucosamine-6-phosphate N-acetyltransferase activity originating from Saccharomyces cerevisiae, the one skilled in the art may refer to the accession number NP_116637 in the UniProt database, or to SEQ ID NO. 28 described herein.

According to another particular embodiment, the one or more recombinant nucleic acids encoding a polypeptide having Glucosamine-6-phosphate N-acetyltransferase activity may be nucleic acid(s) encoding polypeptide having an amino acid sequence selected from the group consisting of sequences having at least 80% amino acid identity with the amino acid sequence set forth as SEQ ID NO. 28, and also a biological activity of the same nature as the amino acid sequence set forth as SEQ ID NO. 28.

A biological activity of the same nature regarding this sequence is as described previously, i.e. the capacity to catalyze the conversion of Glucosamine-6-phosphate into N-acetyl-glucosamine-6-phosphate.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the one or more recombinant nucleic acids encoding a polypeptide having Glucosamine-6-phosphate N-acetyltransferase activity in the present invention is regulated by at least one promoter and at least one terminator, such as herein after defined more in details, which are present in 5′ and 3′ position respectively of the one or more recombinant nucleic acids encoding a polypeptide having Glucosamine-6-phosphate N-acetyltransferase activity.

### Phosphoacetylglucosamine Mutase (PCM1)

The phosphoacetylglucosamine mutase enzyme is a protein which is described in the art for catalyzing the conversion of N-acetyl-glucosamine-6-phosphate into N-acetyl-glucosamine-1-phosphate. The phosphoacetylglucosamine mutase originating from Saccharomyces cerevisiae may be termed PCM1.

A method implemented to measure the activity level of a polypeptide having phosphoacetylglucosamine mutase activity belongs to the general knowledge of the one skilled in the art.

In this regard, the one skilled in the art may advantageously refer to the method described by Bandini et al. (Molecular Microbiology 85 (3), 513-534, 2012).

Preferred polypeptide having phosphoacetylglucosamine mutase activity in the present invention is an enzyme having an EC number of n° 5.4.2.3.

According to a preferred embodiment, the one or more recombinant nucleic acids encoding a polypeptide having phosphoacetylglucosamine mutase activity may originate or be derived from organisms preferably selected in a group consisting of prokaryotic organisms and eukaryotic organisms. In some preferred embodiments, the one or more recombinant nucleic acids encoding a polypeptide having phosphoacetylglucosamine mutase activity may originate or be derived from a yeast, and especially from Saccharomyces cerevisiae.

According to a particular embodiment, the one or more recombinant nucleic acids encoding a polypeptide having phosphoacetylglucosamine mutase activity may be selected from the group consisting of nucleic acid sequences having (i) at least 80% nucleic acid identity with a nucleic acid sequence set forth as SEQ ID NO: 29, and (ii) a biological activity of the same nature as the nucleic acid sequence set forth as SEQ ID NO: 29. The nucleic acid set forth as SEQ ID NO: 29 encodes a polypeptide having phosphoacetylglucosamine mutase activity originating from Saccharomyces, that may also be termed PCM1.

A biological activity of the same nature regarding this sequence is, as previously explained, the capacity to code for a polypeptide that converts N-acetyl-glucosamine-6-phosphate into N-acetyl-glucosamine-1-phosphate.

As described herein, a nucleic acid sequence having at least 80% nucleotide identity with a reference nucleic acid sequence encompasses nucleic acid sequences having at least 81%, 82%, 83%, 84%, 85%, 86%, 87%, 88%, 89%, 90%, 91%, 92%, 93%, 94%, 95%, 96%, 97%, 98% and 99% nucleotide identity with the said reference nucleic acid sequence, and also a biological activity of the same nature as the said reference nucleic acid sequence.

For the amino acid sequence of the polypeptide having phosphoacetylglucosamine mutase activity originating from Saccharomyces cerevisiae, the one skilled in the art may refer to the accession number NP_010856 in the UniProt database, or to SEQ ID NO. 30 described herein.

According to another particular embodiment, the one or more recombinant nucleic acids encoding a polypeptide having phosphoacetylglucosamine mutase activity may be nucleic acid(s) encoding polypeptide having an amino acid sequence selected from the group consisting of sequences having at least 80% amino acid identity with the amino acid sequence set forth as SEQ ID NO. 30, and also a biological activity of the same nature as the amino acid sequence set forth as SEQ ID NO. 30.

A biological activity of the same nature regarding this sequence is as described previously, i.e. the capacity to catalyze the conversion of N-acetyl-glucosamine-6-phosphate into N-acetyl-glucosamine-1-phosphate.

As described herein, an amino acid sequence having at least 80% amino acid identity with a reference amino acid sequence encompasses amino acid sequences having at least 81%, 82%, 83%, 84%, 85%, 86%, 87%, 88%, 89%, 90%, 91%, 92%, 93%, 94%, 95%, 96%, 97%, 98% and 99% amino acid identity with the said reference amino acid sequence, and also a biological activity of the same nature as the said reference amino acid sequence.

As above-mentioned, the expression level of the one or more recombinant nucleic acids encoding a polypeptide having phosphoacetylglucosamine mutase activity in the present invention is regulated by at least one promoter and at least one terminator, such as herein after defined more in detail, which are present in 5′ and 3′ position respectively of the one or more recombinant nucleic acids encoding a polypeptide having phosphoacetylglucosamine mutase activity.

### Promoters

As disclosed herein, the expression of the genes of interest that have been genetically engineered for obtaining a recombinant cell according to the invention comprise appropriate regulatory sequences that are functional in the recombinant cells of the invention, and in particular in recombinant yeast cells of the invention, including in particular Saccharomyces cerevisiae.

Various promoters may be used for the desired expression of the coding sequences of interest.

Promoters according to the invention may be selected from the group consisting of the following promoters:

(SEQ ID NO: 31)
pTDH3,
(SEQ ID NO: 32)
pTDH3.sk,
(SEQ ID NO: 33)
pTDH3-1.Sba,
(SEQ ID NO: 34)
pTDH3.Sar,
(SEQ ID NO: 35)
pENO2,
(SEQ ID NO: 36)
pTEF3,
(SEQ ID NO: 37)
pTEF1,
(SEQ ID NO: 38)
pTEF1.ago,
(SEQ ID NO: 39)
pTEF1.sba,
(SEQ ID NO: 40)
pPDC1,
(SEQ ID NO: 41)
pCCW12,
(SEQ ID NO: 42)
pCCW12.Sm,
(SEQ ID NO: 43)
pCCW12.sk,
(SEQ ID NO: 44)
pCCW12.sba,
(SEQ ID NO: 45)
pCCW12.sar,
(SEQ ID NO: 46)
pNUP57,
(SEQ ID NO: 47)
pCCW10.ago,
(SEQ ID NO: 48)
pCWP2,
(SEQ ID NO: 49)
pCCW120.Sca,
and
(SEQ ID NO: 50)
pRPLA1.

Promoters more particularly interesting in the present invention may be selected from the group consisting of:

(SEQ ID NO: 31)
pTDH3,
(SEQ ID NO: 32)
pTDH3.sk,
(SEQ ID NO: 33)
pTDH3-1.Sba,
(SEQ ID NO: 34)
pTDH3.Sar,
(SEQ ID NO: 35)
pENO2,
(SEQ ID NO: 36)
pTEF3,
(SEQ ID NO: 37)
pTEF1,
(SEQ ID NO: 38)
pTEF1.ago,
(SEQ ID NO: 39)
pTEF1.sba,
(SEQ ID NO: 40)
pPDC1,
(SEQ ID NO: 41)
pCCW12,
(SEQ ID NO: 42)
pCCW12.Sm,
(SEQ ID NO: 43)
pCCW12.sk,
(SEQ ID NO: 44)
pCCW12.sba,
and
(SEQ ID NO: 45)
pCCW12.sar.

Said promoters can in particular be selected from the group consisting of pTDH3, pTDH3-1.Sba, pTDH3.Sar, pENO2, pTEF3, pTEF1, pPDC1, pCCW12, pCCW12.Sm, pCCW12.sk, pCCW12.sba, and pCCW12.sar.

Alternatively, promoters interesting in the present invention may be selected from the group consisting of:

(SEQ ID NO: 46)
pNUP57,
and
(SEQ ID NO: 47)
pCCW10.ago.

Other promoters of interest in the present invention may be:

(SEQ ID NO: 48)
pCWP2;
(SEQ ID NO: 49)
pCCW120.Sca;
and
(SEQ ID NO: 50)
pRPLA1.

As previously mentioned, inducible or repressible promoters are promoters whose activity is controlled by the presence or absence of biotic or abiotic factors and also by the quantity of said factor. Accordingly, for some promoters, their activity will in particular be induced and thus increased when the quantity of a given factor increases or is increased, and, accordingly, the activity of these same promoters can be repressed and thus reduced when the quantity of said factor diminishes or is reduced. The quantity of said factor(s) in the culture medium of a recombinant yeast cell of the invention comprising inducible or repressible promoters can be decided and thus controlled by the man skilled in the art.

For example, increasing the quantity of copper in a culture medium of a recombinant yeast cell according to the invention comprising a pCUP-1 promoter will induce and thus increase transcription of the gene under the control of this promoter. On the contrary, reducing the quantity of copper in said culture medium will lead to a repression, and thus a reduced, transcription of the gene under the control of this promoter.

In another example, increasing the quantity of methionine in a culture medium of a recombinant yeast cell according to the invention comprising a pMET6 promoter will repress and thus decrease transcription of the gene under the control of this promoter. On the contrary, reducing the quantity of methionine in said culture medium will lead to an induced, and thus an increased, transcription of the gene under the control of this promoter.

For this reason, the following promoters are referred to in the present text as being “inducible or repressible promoters”.

According to a first embodiment, inducible or repressible promoters according to the invention may be selected from the group comprising promoters inducible or repressible with copper, promoters inducible or repressible with methionine and promoters inducible or repressible with threonine, and are in particular is pCUP1-copper inducible or repressible.

According to this embodiment, the inducible or repressible promoter according to the invention can in particular be pCUP1 (SEQ ID NO: 51).

The activity of these promoters is thus induced by the increasing presence of methionine, copper or threonine as indicated above, and their activity diminishes, i.e. is repressed, when the quantity of methionine, copper or threonine is reduced.

According to a second embodiment, inducible or repressible promoters according to the invention may be selected from the group comprising promoters inducible or repressible with copper, promoters inducible or repressible with lysine and promoters inducible or repressible with methionine, and in particular selected from the group consisting of:
pMET6-methionine inducible or repressible (SEQ ID NO: 52),
pMET25-methionine inducible or repressible (SEQ ID NO: 53), and
pSAM1-methionine inducible or repressible (SEQ ID NO: 54),

According to this particular embodiment, the inducible or repressible promoter according to the invention may be selected from the group consisting of pMET6, pMET25 and pSAM1.

The activity of these promoters is thus repressed by the increasing presence of methionine, copper, lysine or glucose as indicated above, and their activity increases, i.e. is induced, when the quantity of methionine, copper, lysine or glucose is reduced.

In a particular embodiment, inducible or repressible promoters according to the invention may be selected from the group comprising promoters inducible or repressible with copper, promoters inducible or repressible with glucose, promoters inducible or repressible with lysine, promoters inducible or repressible with methionine and promoters inducible or repressible with threonine.

In a more particular embodiment, the inducible or repressible promoter according to the invention may be selected from the group consisting of pCUP1, pMET6, pSAM1, and pMET25.

In a particular embodiment of the invention, promoters interesting in the present invention may be selected from the group consisting of:

(SEQ ID NO: 31)
pTDH3,
(SEQ ID NO: 37)
pTEF1,
(SEQ ID NO: 38)
pTEF1.ago,
(SEQ ID NO: 40)
pPDC1,
(SEQ ID NO: 41)
pCCW12,
(SEQ ID NO: 43)
pCCW12.sk,
(SEQ ID NO: 44)
pCCW12.sba,
(SEQ ID NO: 45)
pCCW12.sar,
(SEQ ID NO: 48)
pCWP2,
(SEQ ID NO: 49)
pCCW120.Sca,
(SEQ ID NO: 51)
pCUP1,
(SEQ ID NO: 52)
pMET6,
and
(SEQ ID NO: 54)
pSAM1.

Synthetic promoters as described in Blazeck & Alper (2013) Biotechnol. J. 8 46-58 can also be used.

The promoters of the invention may originate from any organism from the Saccharomyceses class and may in particular originate from an organism selected from the group consisting of at least one of Saccharomyces cerevisiae, Saccharomyces boulardii, Saccharomyces castelii, Saccharomyces bayanus, Saccharomyces arboricola, Saccharomyces kudriavzevii, Saccharomyces mikatae, Ashbya gossypii, Kluveromyces lactis, Pichia pastoris, Candida glabrata, Candida tropicalis, Debaryomyces castelii, Yarrowia lipolitica and Cyberlindnera jadinii.

The promoters of the invention can preferably originate from an organism selected from the group consisting of Saccharomyces cerevisiae (sc), Saccharomyces mikatae (Sm), Saccharomyces kudriavzevii (sk), Saccharomyces bayanus (sba), and Saccharomyces arboricola (Sar).

### Terminators

As it is disclosed herein, the expression of the genes of interest that have been genetically engineered for obtaining a recombinant cell according to the invention, and in particular a recombinant yeast according to the invention, comprise appropriate transcription terminator sequences that are functional in recombinant cells of the invention, and in particular in recombinant yeast cells of the invention, in particular in Saccharomyces cerevisiae.

Said transcription terminators, identical or different, may be found in literature Yamanishi et al., (2013) ACS synthetic biology 2, 337-347.

Terminators more particularly interesting in the present invention may be selected from the group comprising:
tTPI1 from the gene encoding for the Triose Phosphate Isomerase (SEQ ID NO: 55),
tMET25 from the gene encoding for the O-acetyl homoserine-O-acetyl serine sulfhydrylase (SEQ ID NO: 56),

(SEQ ID NO: 57)
tDIT1,
(SEQ ID NO: 58)
tRPL3,
(SEQ ID NO: 59)
tRPL3.sm,
(SEQ ID NO: 60)
tRPL3.sba,
(SEQ ID NO: 61)
tRPL41B,
(SEQ ID NO: 62)
tRPL15A,
(SEQ ID NO: 63)
tRPL15A.sba,
(SEQ ID NO: 64)
tIDP1,
and
(SEQ ID NO: 65)
tTEF1.sba.

In particular, said terminator may be selected from the group consisting of tTPI1, tMET25, tDIT1, tRPL3, tRPL3.sm, tRPL3.sba, tRPL41B, tRPL15A, tRPL15A.sba, tIDP1 and tTEF1.sba.

The terminators of the invention can originate from any organism from the Saccharomyceses class and may in particular originate from an organism selected from the group consisting of Saccharomyces cerevisiae and Saccharomyces Bayanus.

### Recombinant Yeast

Recombinant cells of the invention can be selected from the group consisting of yeasts and bacteria.

Recombinant cells of the invention, such as a recombinant host cell of the invention, are preferably recombinant yeast cells.

Generally, yeast can grow rapidly and can be cultivated at higher density as compared with bacteria, and does not require an aseptic environment in the industrial setting. Furthermore, yeast cells can be more easily separated from the culture medium compared to bacterial cells, greatly simplifying the process for product extraction and purification.

A recombinant cell of the invention, and in particular a recombinant yeast cell of the invention is preferably a Saccharomycetales cell.

A recombinant cell of the invention, and in particular a recombinant yeast of the invention can in particular belong to the Saccharomyces genus, or to the Candida genus, or to the Kluyveromyces genus, or to the Ogataea genus, or to the Yarrowia genus, or to the Debaryomyces genus, or to the Ashbya genus.

A recombinant cell of the invention belonging to the Saccharomyces genus can be selected from the group consisting of Saccharomyces cerevisiae, Saccharomyces boulardii, Saccharomyces bayanus, Saccharomyces paradoxus, Saccharomyces mikatae, Saccharomyces castelli, Saccharomyces cariocanus, Saccharomyces kudriavzevii, Saccharomyces arboricolus, Saccharomyces pastorianus, Saccharomyces uvarum and Saccharomyces delbrueckii.

A recombinant cell of the invention belonging to the Candida genus can be selected from the group consisting of Candida albicans, Candida glabrata, Candida tropicalis, Candida dubliniensis, Candida parapsilosis, Candida lusitaniae and Candida guilliermondii.

A recombinant cell of the invention belonging to the Kluyveromyces genus can be selected from the group consisting of Kluyveromyces lactis, Kluyveromyces marxianus, Kluyveromyces polysporus, Kluyveromyces thermotoleren, Kluyveromyces dobzhanskii and Kluyveromyces wickerhamii.

A recombinant cell of the invention belonging to the Ogataea genus can be selected from the group consisting of Ogataea polymorpha, Ogataea histrianica, Ogataea deakii, Ogataea kolombanensis, Ogataea philodendra, Ogataea siamensis, Ogataea angusta, Ogataea parapolymorpha, Ogataea minuta, Ogataea nonfermentans and Ogataea kodamae.

A recombinant cell of the invention belonging to the Yarrowia genus can be selected from the group consisting of Yarrowia lypolytica, Yarrowia parophonii, Yarrowia galli, Yarrowia oslonensis, Yarrowia alimentaria, Yarrowia hollandica and Yarrowia yakushimensis.

A recombinant cell of the invention belonging to the Debaryomyces genus can be selected from the group consisting of Debaryomyces hansenii, Debaryomyces carsonii, Debaryomyces castellii, Debaryomyces marama, Debaryomyces occidentalis, Debaryomyces oviformis, Debaryomyces nepalensis, Debaryomyces coudertii, Debaryomyces udenii, Debaryomyces psychrosporus and Debaryomyces yamadae.

A recombinant cell of the invention belonging to the Ashbya genus can be selected from the group consisting of Ashbya gossypii and Ashbya aceri.

A recombinant cell of the invention, and in particular a recombinant yeast cell of the invention, can in particular be selected from the group consisting of Saccharomyces cerevisiae, Saccharomyces boulardii, Saccharomyces bayanus, Saccharomyces paradoxus, Saccharomyces mikatae, Saccharomyces castelli, Candida albicans, Candida glabrata, Candida tropicalis, Kluyveromyces lactis, Kluyveromyces marxianus, Kluyveromyces polysporus, Kluyveromyces thermotolerens, Ogataea polymorpha, Yarrowia lypolytica, Debaryomyces hansenii, and Ashbya gossypii, and is preferably Saccharomyces cerevisiae.

In a particular embodiment, the recombinant yeast cell according to the invention is of the genus Saccharomyces, Kluyveromyces, or Eremothecium, more particularly is of the species selected from the group consisting of Saccharomyces cerevisiae, Kluyveromyces Marxianus, Ogataea polymorpha and Ashbya gossypii.

In an embodiment, a recombinant host cell of the invention is a yeast selected belonging to the Saccharomycesales order, in particular to the Saccharomycesaceae family, and is in particular selected from the group consisting of Yarrowia lipolitica, Kluyveromyces Marxianus, Ogataea polymorpha, Ashbya gossypii and Saccharomyces cerevisiae.

A recombinant cell of the invention can most preferably be a Saccharomyces cerevisiae cell.

As above-mentioned, a recombinant cell according to the invention has the ability to produce heparosan by insertion of the one or more recombinant nucleic acids according to the present invention. In certain embodiments, a recombinant yeast according to the invention has the ability to produce heparosan having a controlled size by insertion of the one or more recombinant nucleic acids according to the present invention.

Methods implemented to insert a specific DNA construct within a gene belong to the general knowledge of a man skilled in the art. A related method is described in more details in the herein after examples.

However, unexpected technical difficulties were encountered because the consequences of the insertion of DNA constructs within the genome of a cell, and in particular within the genome of a yeast, such as for example in the genome of Saccharomyces cerevisiae, are unpredictable. In particular, the survival rate of the cells, and in particular of the yeasts, and their ability to grow and produce the desired heparosan is also unpredictable.

In order to obtain a recombinant cell, and in particular a recombinant yeast, of the invention, many different constructs were tested by the inventors in order to obtain a viable and efficient recombinant cell, and in particular yeast cell.

### Culture Conditions

The present invention also relates to the use of a recombinant cell of the invention for the production of heparosan, in particular of heparosan of controlled molecular weight.

The present invention further relates to a method of producing heparosan, in particular heparosan of a desired molecular weight, comprising:
(a) culturing a recombinant cell according to the invention in a cultivation medium for a time sufficient to produce heparosan; and
(b) optionally isolating or recovering the heparosan from the recombinant cell and/or from the cultivation medium.

Typically, cells of the invention, and in particular yeasts of the invention, are grown at a temperature in the range of about 20° C. to about 37° C., preferably at a temperature ranging from 27 to 34° C., in an appropriate culture medium.

Suitable growth media for cells of the invention, and in particular for yeasts of the invention, are common commercially prepared media such as broth that includes yeast nitrogen base, ammonium sulfate, and dextrose as the carbon/energy source or YPD Medium, a blend of peptone, yeast extract, and dextrose in optimal proportions for growing most. Other defined or synthetic growth media may also be used and the appropriate medium for growth of the particular cell, and in particular yeast, will be known by one skilled in the art of microbiology or fermentation science.

A particular medium that is suitable herein is the SY medium which comprises the following elements:

KH2PO4: 100 mM; MgSO4 7H2O: 2.8 mM; K2SO4: 11.5 mM; Na2SO4: 1.1 mM; NaCl: 2.6 mM; CaCl2 2H2O: 0.7 mM; CuSO4 5H2O: 15 μM; KI: 6 M; FeC13: 30 M; ZnSO4 7H2O: 61 μM; MnSO4 H2O: 25 μM; H2SO4: 110 μM; Panthotenic Acids hemicalcium salt: 42 μM; Thiamin hydrochloride: 59 μM; Pyridoxin hydrochloride: 49 μM; Myo-Inositol (C6H12O6): 555 μM; Nicotinic acid (C6H5NO2): 29 μM; D-Biotine: 0.82 μM; Ammonium citrate tribasic: 33 mM; and glucose or sucrose 2-30%.

Carbon sources that may be used in the culture medium include fructose, mannose, xylose and arabinose; oligosaccharides such as lactose, maltose, galactose, or sucrose; polysaccharides such as starch or cellulose or mixtures thereof; and unpurified mixtures from renewable feedstocks such as cheese whey permeate cornsteep liquor, sugar beet molasses, and barley malt.

Nitrogen sources that may be included in the culture medium include peptone, yeast extracts, meat extracts, malt extracts, urea, ammonium sulfate, ammonium chloride, ammonium nitrate, ammonium phosphate, ammonium citrate and combinations thereof.

The culture medium may further comprise trace elements (e.g., metal salts), for example magnesium salts, cobalt salts and/or manganese salts; as well as growth factors such as amino acids, vitamins, growth promoters, and the like.

Examples of vitamins that may be included are Panthotenic Acids hemicalcium, Thiamin hydrochloride; Pyridoxin hydrochloride; Myo-Inositol; Nicotinic acid; D-Biotine, folic acid, p-aminobenzoïque acid, riboflavin.

A culture medium of the invention may further include rare elements, such as CuSO₄·5H₂O, KI, FeCl₃, ZnSO₄·7H₂O, MnSO₄·H₂O, or H₂SO₄, MgCl₂, CaCl₂, NaCl, K₂HPO₄, KH₂PO₄, ZnCl, H₃BO₃, MnSO₄, Na₂MoO₄.

The term “appropriate culture medium” is above-defined.

Examples of known culture media for a recombinant cell according to the present invention are known to the person skilled in the art, and are presented in the following publication D. Burke et al., Methods in yeast Genetics—A cold spring harbor laboratory course Manual (2000).

Suitable pH ranges for the fermentation may be between pH 3.0 to pH 7.5, where pH 4 to pH 6 is preferred as the initial condition.

As mentioned elsewhere in this specification, the pH value of the culture medium can be regulated during the cultivation step of a method of the invention in order to modulate the activity of the polypeptide having glucuronosyl-disulfoglucosamine glucuronidase activity, which will impact the molecular weight of the heparosan molecules produced by the recombinant cell of the invention, and in particular by the recombinant yeast of the invention.

In particular, the pH of the culture medium can be modified according to the heparosan that is meant to be produced by the recombinant yeast. For example, the pH of the culture medium may remain at a pH of 4, of 5.5 or of 6 during the culture time.

In a particular embodiment, the pH of the culture medium may change or be changed during the time length of the culture of the recombinant cell of the invention, in particular of the recombinant yeast of the invention. As previously mentioned, Saccharomyces cerevisiae acidifies the medium in which it is cultivated and thus reduces the pH of its culture medium. For example, the pH of the culture medium may begin at 6, may be taken down to 4 and then brought back up to 6. In another example, the pH of the culture medium may begin at 6, remain or be maintained at 6, and then may be lowered to 4.

In a particular embodiment, the pH of the culture medium may be modulated during the cultivating step (a) of a method of the invention so that at the end of the cultivating step of a method of the invention, the pH of the cultivation medium is the same as the pH at the beginning of said cultivation step (a).

In another embodiment, the pH of the culture medium may remain the same during the time length of the culture of the recombinant cell of the invention, and in particular the recombinant yeast cell of the invention.

Said time length of the culture of a recombinant cell according to the invention, and in particular of the recombinant yeast cell of the invention, can vary depending on the molecular weight of the heparosan of interest. The longer said time length, the lower the molecular weight of the heparosan in a given culture medium of a recombinant cell of the invention, and in particular a recombinant yeast cell of the invention.

The time length of the culture time of a recombinant cell of the invention, and in particular a recombinant yeast in the present invention can be a period of from about 35 hours to about 50 hours, preferably from about 40 hours to about 50 hours, and is in particular about 48 hours.

Fermentations may be performed under aerobic conditions or micro-aerobic conditions.

The amount of heparosan product in the fermentation medium can be determined using a number of methods known in the art, for example, high performance liquid chromatography (HPLC) or gas chromatography (GC).

The present process may employ a batch method of fermentation. A classical batch fermentation is a closed system where the composition of the medium is set at the beginning of the fermentation and not subject to artificial alterations during the fermentation. Thus, at the beginning of the fermentation, the medium is inoculated with the desired organism or organisms, and fermentation is permitted to occur without adding anything to the system. Typically, however, a “batch” fermentation method or system is batch with respect to the addition of carbon source and attempts are often made at controlling factors such as temperature, pH and oxygen concentration. In batch systems, the metabolite and biomass compositions of the system change constantly up to the time when the fermentation is stopped. Within batch cultures cells progress through a static lag phase to a high growth log phase and finally to a stationary phase where growth rate is diminished or halted. If untreated, cells in the stationary phase will eventually die. Cells in log phase generally are responsible for the bulk of production of end product or intermediate.

A Fed-Batch system may also be used in the present invention. A Fed-Batch system is similar to a typical batch system with the exception that the carbon source substrate is added in increments as the fermentation progresses. Fed-Batch systems are useful when catabolite repression (e.g. glucose repression) is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂.

Batch and Fed-Batch culturing methods are common and well known in the art and examples may be found in Biotechnology: A Textbook of Industrial Microbiology, Crueger, Crueger, and Brock, Second Edition (1989) Sinauer Associates, Inc., Sunderland,

MA, or Deshpande, Mukund V., Appl. Biochem. Biotechnol., 36, 227, (1992). Although the present invention is performed in batch mode it is contemplated that the method would be adaptable to continuous fermentation.

Continuous fermentation is an open system where a defined fermentation medium is added continuously to a bioreactor and an equal amount of conditioned media is removed simultaneously for processing. Continuous fermentation generally maintains the cultures at a constant high density where cells are primarily in log phase growth.

Continuous fermentation allows for the modulation of one factor or any number of factors that affect cell growth or end product concentration. For example, one method will maintain a limiting nutrient such as the carbon source or nitrogen level at a fixed rate and allow all other parameters to vary. In other systems a number of factors affecting growth can be altered continuously while the cell concentration, measured by media turbidity, is kept constant. Continuous systems strive to maintain steady state growth conditions and thus the cell loss due to the medium being drawn off must be balanced against the cell growth rate in the fermentation. Methods of modulating nutrients and growth factors for continuous fermentation processes as well as techniques for maximizing the rate of product formation are well known in the art of industrial microbiology.

It is contemplated that the present invention may be practiced using either batch, fed-batch or continuous processes and that any known mode of fermentation would be suitable. Additionally, it is contemplated that cells may be immobilized on a substrate as whole cell catalysts and subjected to fermentation conditions for production.

In order to still improve the heparosan production, a particular embodiment may consist of culturing the recombinant cells of the invention, in particular the recombinant yeast cells of the invention, in an appropriate culture medium, such as above-mentioned, wherein the said culture medium comprises an optimal amount of carbon source, especially glucose or sucrose.

In preferred embodiments, the carbon source comprised in said optimal culture medium consists of glucose and/or sucrose. In preferred embodiments, the said optimal culture medium comprises 1% w/w or more glucose and/or sucrose, in particular comprises 5% w/w or more glucose and/or sucrose, in particular comprises 10% w/w or more glucose and/or sucrose, in particular comprises 15% w/w or more glucose and/or sucrose. In preferred embodiments, the said optimal culture medium comprises at most 40% w/w glucose, which includes at most 35% w/w glucose.

In a preferred embodiment, the method of the invention is carried out on an industrial scale.

More particularly, the cultivation medium of the method according to the invention can be at least about 100 L, more preferably in the range of about 1000 L to about 3000 L, even more preferably about 10,000 L, even more preferably 100,000 L, or even about 250,000 L.

The invention further relates to a method for producing heparosan as previously described, and comprising the steps of:
(a) culturing a recombinant cell of the invention in a culture medium; and
(b) recovering the heparosan from said culture medium,
wherein the heparosan recovered in step (b) has a molecular weight controlled through the selection of:
   the nature and origin of the one or more recombinant nucleic acids encoding a polypeptide having glucuronosyl-disulfoglucosamine glucuronidase activity of the recombinant cell of the invention, and in particular of the recombinant yeast of the invention,
   the nature and origin of the promoter controlling the expression of the one or more recombinant nucleic acids encoding a polypeptide having glucuronosyl-disulfoglucosamine glucuronidase activity of the recombinant cell of the invention, and in particular of the recombinant yeast of the invention,
   the presence or absence of an anchoring signal associated to the one or more recombinant nucleic acids encoding a polypeptide having glucuronosyl-disulfoglucosamine glucuronidase activity of the recombinant cell of the invention, and in particular of the recombinant yeast of the invention,
   the pH of the culture medium during the step of culturing the recombinant cell of the invention, and in particular of the recombinant yeast of the invention, and/or
   the duration of the culturing of the recombinant cell of the invention, and in particular of the recombinant yeast of the invention.

The molecular weight of the heparosan can be a particular molecular weight or more preferably a specific range of molecular weights such as, for example, less than 50 kDa, in the range of about 20 kDa to about 50 kDa, greater than or equal to 50 kDa, in the range of about 50 kDa to about 150 kDa, in the range of about 50 kDa to about 250 kDa, greater than or equal to 100 kDa, in the range of about 100 kDa to about 1500 kDa, in the range of about 150 kDa to about 1500 kDa, greater than 1000 kDa or greater than 1500 kDa.

The present invention also relates to the use of a recombinant cell according to the invention, and in particular a recombinant yeast cell of the invention, for the production of heparosan having a molecular weight in the range of from about 20 kDa to about 50 kDa or from about 50 kDa to about 1000 kDa.

The secretion signal of the present invention may for example have:
the nucleic acid sequence set forth as SEQ ID NO: 66; and/or
the amino acid sequence set forth as SEQ ID NO: 67.

The anchoring signal of the present invention may for example have:
the nucleic acid sequence set forth as SEQ ID NO: 68; and/or
the amino acid sequence set forth SEQ ID NO: 69.

The secretion signal may be fused to the polypeptide having glucuronosyl-disulfoglucosamine glucuronidase activity by creating a chimeric nucleic acid starting by a nucleic acid sequence encoding the signal peptide followed by a recombinant nucleic acid encoding a polypeptide having glucuronosyl-disulfoglucosamine glucuronidase activity as previously defined.

The secretion signal and anchoring signal may be fused to the polypeptide having glucuronosyl-disulfoglucosamine glucuronidase activity by creating a chimeric nucleic acid starting by a nucleic acid sequence encoding the signal peptide followed by a recombinant nucleic acid encoding a polypeptide having glucuronosyl-disulfoglucosamine glucuronidase activity as previously defined, followed by a nucleic acid sequence encoding for the anchoring signal.

Such chimeric nucleic acid sequences may be obtained by techniques known by the man skilled in the art such as chemical synthesis of nucleic acids or any recombination techniques such as cloning or PCR.

### Purification of Heparosan

According to a specific aspect of the invention, the fermentative production of heparosan comprises a step of isolation of the heparosan from the culture medium. Recovering the heparosan from the culture medium is a routine task for a man skilled in the art. It may be achieved by a number of techniques well known in the art including but not limiting to pervaporation, selective precipitation, filtration, centrifugation, spray drying, lyophylisation or liquid extraction. The expert in the field knows how to adapt parameters of each technique dependant on the characteristics of the material to be separated.

The yeast as model of a cell in the present invention is preferred in that the synthesized heparosan is/are entirely exported outside the cells, thus simplifying the purification process.

Gas stripping is achieved with a stripping gas chosen among helium, argon, carbon dioxide, hydrogen, nitrogen or mixture thereof.

Liquid extraction is achieved with organic solvent as the hydrophobic phase such as pentane, hexane, heptane or dodecane. Renewal solvents may also be used.

### Derivatives

The term “heparosan”, as used herein, is also intended to cover derivatives of heparosan, such as but not limited to heparin (sulfated heparosan) useful for applications in a cosmetic product, a flavor product, a fragrance product, a food product, a food, a beverage, a texturant, a pharmaceutical composition, a dietary supplement, a nutraceutical, a cleaning product, and/or a dental and/or an oral hygiene composition or combinations thereof.

As used herein, “derivatives of heparosan” are heparosan compounds which have been modified with chemical or enzymatical methods and therefore comprise a similar structure to heparosan. These derivatives may display the same or different biological activities as heparosan.

Suitable heparosan derivatives include but are not limited to heparin, enoxaparin, dalteparin, and tinzaparin.

Heparin is useful as an anticoagulant (blood thinner) and in alleviating the risk of reducing, preventing and/or treating heart attacks and unstable angina.

In particular, Heparin is generally used for anticoagulation purposes in order to alleviate the risk of, reduce, prevent and/or treat conditions such as, but not limited to:
Acute coronary syndrome, e.g., NSTEMI;
Atrial fibrillation;
Deep-vein thrombosis and pulmonary embolism;
Cardiopulmonary bypass for heart surgery;
ECMO circuit for extracorporeal life support;
Hemofiltration; and/or
Indwelling central or peripheral venous catheters.

Heparin and its low-molecular-weight derivatives (e.g., enoxaparin, dalteparin, tinzaparin) are effective in preventing deep vein thromboses and pulmonary emboli in people at risk.

Heparin is given by injection into a vein or under the skin.

Other uses include applications inside test tubes and kidney dialysis machines.

Heparosan, in particular Heparin, may be prepared as a composition.

### Formulations and Products

A composition of the invention may be incorporated into a formulation/product, such as a nutraceutical, veterinary, oenological or cosmetic formulation/product.

Thus, the present invention provides a formulation comprising a composition of the invention. For example, the present invention may provide a cosmetic formulation comprising the composition of the invention.

The present invention also provides a product comprising a composition of the invention. For example, the present invention may provide a cosmetic product comprising the composition of the invention.

A “cosmetic product” is intended to mean any substance or mixture intended to be placed in contact with the external parts of the human body (epidermis, hair system, nails, lips and external genital organs) or with the teeth and the mucous membranes of the oral cavity with a view exclusively or mainly to cleaning them, perfuming them, changing their appearance, protecting them, keeping them in good condition, correcting body odours and/or combinations thereof.

A “substance” is intended to mean a chemical element and its compounds in the natural state or obtained by any manufacturing process, including any additive necessary to preserve its stability and any impurity deriving from the process used but excluding any solvent which may be separated without affecting the stability of the substance or changing its composition.

A “mixture” is intended to mean a mixture or solution composed of two or more substances.

The present invention also provides the use of a composition of the invention in a nutraceutical, pharmaceutical, veterinary, oenological or cosmetic formulation/product.

Such formulations or products are hereinafter referred to as the “formulations or products of the invention”.

The nutraceutical, pharmaceutical, veterinary, oenological or cosmetic formulation/product may optionally further comprise pharmaceutically/veterinary/cosmetic (including cosmetic active) ingredients, such as excipients, carriers and mixtures thereof as appropriate.

“Cosmetic” or “Cosmetic Active Ingredients” means any and all natural, naturally occurring, nature identical, synthetic synthetically produced, biosynthetically produced, sustainable, renewable and/or biodegradable compounds, ingredients, intermediates, molecules, substances, raw materials or products individually or as part of a mixture of compounds, ingredients, intermediates, molecules, substances, raw materials or products, blends, compositions, formulations (including but not limited to skin moisturizers, creams, balms, serums, oils, eye, facial makeup, wash off hair products, leaveon hair products, hair colorants (including but not limited to natural hair colorants) and/or combinations thereof), finished products and related technologies including, but not limited to components, incorporated, for example, into a cosmetic formulation (such as but not limited to natural colorants, preservatives, emulsifiers, anti-oxidants and the like which do not, for example, have an activity on the skin, hair, scalp and the like but play a role in the formulation of the finished product), delivery systems, marketing aids (such as, for example, coloured unispheres applied to translucent formulations) and methods of making anything related thereto useful in/used for/intended for:
application by rubbing, pouring, sprinkling, spraying or otherwise directly on or to a human or animal body and/or by placing in contact with the various external and/or on surface parts of a human or animal body (including but not limited to the skin, hair, body hair, the hair system, scalp, nails, lips, external genitalia, teeth, oral and/or nasal mucosa and the like); and/or application indirectly on or to a human or animal body such as for example, application as part of a textile or application to a textile as part of a delivery device (such as a capsule) or a delivery system (such as blends or formulations) applied to a textile; and/or
cleansing, caring, cooling, beautifying, conditioning, treating, soothing, texturizing, promoting attractiveness, protecting, maintaining, improving, enhancing, altering and/or changing an external part and/or surface of a human or animal body (such as, but not limited to the scalp) or the aesthetic appearance of a human or animal body; and/or with a view to mainly cleaning or perfuming, or protecting or maintaining in good condition or combating body odour or changing the appearance of or correcting or repairing a state of imbalance in skin, oral mucosa, scalp or hair by providing a calming, healing, repairing, or revitalization, hydration of the skin or in order to provide relief to, lubricate, moisten, tone, heal, sterilize, relieve, correct and/or remedy states of dryness, irritation, injury or fatigue, and/or with a view to correcting pigmentation disorders or providing a non-pharmaceutical prevention and/or treatment of dandruff, acne, irritation and/or inflammation and the like and/or rebalancing the bacterial flora (such as, for example, the microbiome) on the surface of the skin (such as, for example, by promoting the level of beneficial bacterial flora on the skin surface) and/or for the purposes of keeping a human or animal body in good condition for health and/or wellbeing purposes and/or for improving the appearance of a human or animal body by, for example, improving the appearance of a product applied to a human or body; and/or
providing a cosmetic and/or dermatological function and/or benefit with a biological activity benefit (but without affecting a body's structure or function. For the avoidance of doubt, a Cosmetic or Cosmetic Active Ingredient or any part thereof may also qualify as a Functional Ingredient and/or Nutraceutical.

“Functional Ingredient” means a food ingredient or part of a food that provides medicinal or health benefits, including any of the following: a carotenoid, dietary fiber, fatty acid, saponin, antioxidant, flavonoid, isothiocyanate, phenol, polyphenol (such as resveratrol), plant sterol or stanol (phytosterols and phytostanols), a polyol, a prebiotic, a phytoestrogen, soy protein, sulfides/thiol, a vitamin, glucosamine, preservatives, hydration agents, edible gelling ingredients, edible gel mixes and gel compositions, long chain primary aliphatic saturated alcohols, colour agents, texturizing agents, emulsifiers and combinations thereof.

“Nutraceutical” means any and all natural, naturally occurring, sustainable, synthetically-produced and biosynthetically-produced compounds, mixtures of compounds, Functional Ingredients, molecules, compositions, raw materials, and intermediates (including components and delivery devices (such as capsules) related thereto, delivery systems thereof (such as blends or formulations) and methods of making the foregoing) that are associated with health and/or cosmetic benefits, as well as improving or maintaining the appearance of the human body. For the avoidance of doubt, Nutraceuticals includes compounds that can be used as supplements to food or beverage, whether a solid formulation, capsule, tablet, liquid formulation, solution or suspension.

Alternatively, the nutraceutical, pharmaceutical, veterinary, oenological or cosmetic formulation/product may consist or consist essentially of the composition of the invention.

The cosmetic formulation/product may be an anti-aging formulation.

As used herein, references to pharmaceutically, veterinary or cosmetically acceptable excipients may refer to pharmaceutically, veterinary or cosmetically acceptable adjuvants, diluents and/or carriers as known to those skilled in the art.

By “pharmaceutically/veterinary/cosmetically acceptable” we mean that the additional components of the composition are generally safe, non-toxic, and neither biologically nor otherwise undesirable. For example, the additional components may be generally sterile and pyrogen free. Such components must be “acceptable” in the sense of being compatible with the composition of the invention and not deleterious to the recipients thereof. Thus, “pharmaceutically acceptable excipients” includes any compound(s) used in forming a part of the formulation that is intended to act merely as an excipient, i.e. not intended to have biological activity itself.

The nutraceutical, pharmaceutical, veterinary, oenological or cosmetic formulation/product may be in the form of a liquid or a solid.

Liquid dosage formulations/products for oral administration include solutions, emulsions, aqueous or oily suspensions, syrups and elixirs.

Formulations and products (e.g. pharmaceutical, veterinary or cosmetic formulations/products) described herein, such as those intended for oral administration, may be prepared according to methods known to those skilled in the art, such as by mixing the components of the formulation/product together.

The formulation or product (e.g. pharmaceutical, veterinary or cosmetic formulation/product) may contain one or more additional ingredients, such as pharmaceutical ingredients and excipients, such as sweetening agents, flavouring agents, colouring agents and preserving agents.

The formulation or product (e.g. pharmaceutical, veterinary or cosmetic formulation/product) may also contain one or more additional active ingredients, such as cosmetic or pharmaceutical active ingredients, such as hyaluronic acid, centella asiatica extract, peptides such as Matrixyl® and Argireline®, and mixtures thereof.

The formulation or product of the invention may contain the active ingredient(s) in admixture with non-toxic pharmaceutically acceptable excipients (or ingredients). These excipients (or ingredients) may, for example, be: inert diluents, such as calcium carbonate, sodium carbonate, lactose, calcium phosphate or sodium phosphate; granulating and disintegrating agents, for example, corn starch, maltodextrin or alginic acid; binding agents, for example, starch, gelatine or acacia; or lubricating agents, for example magnesium stearate, stearic acid, talc and mixtures thereof.

Liquid formulations or products (e.g. pharmaceutical, veterinary or cosmetic formulations/products) may be contained within a capsule, which may be uncoated or coated as defined above.

Suitable pharmaceutical or veterinary carriers include inert solid diluents or fillers, sterile aqueous solutions and various organic solvents. Examples of liquid carriers are syrup, peanut oil, olive oil, phospholipids, fatty acids, fatty acid amines, polyoxyethylene and water.

Moreover, the carrier or diluent may include any sustained release material known in the art, such as glyceryl monostearate or glyceryl distearate, alone or mixed with a wax.

Suitable pharmaceutical carriers include inert sterile aqueous solutions and various organic solvents. Examples of liquid carriers are syrup, vegetables oils, phospholipids, fatty acids, fatty acid amines, polyoxyethylene and water. Moreover, the carrier or diluent may include any sustained release material known in the art, such as glyceryl monostearate or glyceryl distearate, alone or mixed with a wax.

Suitable cosmetic carriers are typically those that are suitable for topical administration to the outer surface of the human body, such as the skin and/or hair and/or scalp.

Typically, such carriers are dermatologically acceptable.

The phrase “dermatologically acceptable carrier” means that the carrier is suitable for topical application to the keratinous tissue, has good aesthetic properties, is compatible with the actives in the composition, and will not cause any unreasonable safety or toxicity concerns.

The carrier can be in a wide variety of forms. In some instances, the solubility or dispersibility of the components (e.g. extracts, sunscreen active, additional components) may dictate the form and character of the carrier. Non-limiting examples include simple solutions (e.g. aqueous or anhydrous), dispersions, emulsions, and solid forms (e.g. gels, sticks, flowable solids, or amorphous materials).

The dermatologically acceptable carrier may be in the form of an emulsion. An emulsion may be generally classified as having a continuous aqueous phase (e.g. oil-in-water and water-in-oil-in-water) or a continuous oil phase (e.g. water-in-oil or oil-in-water). The oil phase of the present invention may comprise silicone oils, non-silicone oils such as hydrocarbon oils, esters, ethers, and the like, and mixtures thereof. The aqueous phase typically comprises water and water-soluble ingredients (e.g. water-soluble moisturizing agents, conditioning agents, anti-microbials, humectants and/or other skin care actives). However, in some instances, the aqueous phase may comprise components other than water, including but not limited to watersoluble moisturizing agents, conditioning agents, antimicrobials, humectants and/or other watersoluble skin care actives. In some instances, the non-water component of the composition comprises a humectant, such as glycerin and/or other polyol(s). Emulsions may also contain an emulsifier. Emulsifiers may be non-ionic, anionic or cationic.

The carrier may contain one or more dermatologically acceptable, hydrophilic diluents. As used herein, “diluent” includes materials in which the composition of the invention can be dispersed, dissolved, or otherwise incorporated. Hydrophilic diluents include water, organic hydrophilic diluents, such as lower monovalent alcohols (e.g., C1-C4), and low molecular weight glycols and polyols, including propylene glycol, polyethylene glycol, polypropylene glycol, glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

The cosmetic formulation/product may optionally include one or more additional ingredients commonly used in cosmetic compositions (e.g., colorants, skin tone agents, skin anti-aging agents, anti-inflammatory agents, sunscreen agents, combinations of these and the like), provided that the additional ingredients do not undesirably alter the anti-glycation benefits provided by the composition.

In some instances, it may be desirable to select skin tone agents that function via different biological pathways so that the actives do not interfere with one another, which could reduce the efficacy of both agents. The additional ingredients, when incorporated into the composition, should be suitable for use in contact with human skin tissue without undue toxicity, incompatibility, instability, allergic response, and the like.

The term “carrier” as used herein, may also refer to a natural product or a product originating from nature that has been transformed or modified so that it is distinct from the natural product from which it originated, such as maltodextrin.

The amount of the composition of the invention present in nutraceutical, pharmaceutical, veterinary, oenological or cosmetic formulations or products will vary depending on the application.

Typically, the amount of composition of the invention that may be present in nutraceutical, pharmaceutical, veterinary, oenological or cosmetic formulations or products will be from about 0.001 to about 50% by weight, such as from about 0.01% to about 30% or from about 1% to about 20% of the nutraceutical, pharmaceutical, veterinary, oenological or cosmetic formulations or products, such as from about 0.01 to about 20%, or from about 0.1 to 10% or from about 1 to about 5% by weight of the formulation or product.

Those of skill in the art will recognize that, due to the degenerate nature of the genetic code, a variety of DNA molecules differing in their nucleotide sequences can be used to encode a given enzyme of the disclosure. The native DNA sequence encoding the biosynthetic enzymes described above are referenced herein merely to illustrate an embodiment of the disclosure, and the disclosure includes DNA molecules of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

Described herein are specific genes and proteins useful in the methods, compositions and organisms of the disclosure; however it will be recognized that absolute identity to such genes is not necessary. For example, changes in a particular gene or polynucleotide comprising a sequence encoding a polypeptide or enzyme can be performed and screened for activity. Typically such changes comprise conservative mutations and silent mutations. Such modified or mutated polynucleotides and polypeptides can be screened for expression of a functional enzyme using methods known in the art.

Due to the inherent degeneracy of the genetic code, other polynucleotides which encode substantially the same or functionally equivalent polypeptides can also be used to clone and express the polynucleotides encoding such enzymes.

Techniques known to those skilled in the art may be suitable to identify additional homologous genes and homologous enzymes. Generally, analogous genes and/or analogous enzymes can be identified by functional analysis and will have functional similarities.

Techniques known to those skilled in the art may be suitable to identify analogous genes and analogous enzymes or any biosynthetic pathway genes, proteins, or enzymes, techniques may include, but are not limited to, cloning a gene by PCR using primers based on a published sequence of a gene/enzyme of interest, or by degenerate PCR using degenerate primers designed to amplify a conserved region among a gene of interest. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity (e.g. as described herein or in Kiritani, K., Branched-Chain Amino Acids Methods Enzymology, 1970), then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, or MetaCYC. The candidate gene or enzyme may be identified within the above-mentioned databases in accordance with the teachings herein.

The terms “between . . . and . . . ” and “ranging from . . . to . . . ” should be understood as being inclusive of the limits, unless otherwise specified. Throughout this specification and the claims which follow, unless the context requires otherwise, the word “comprise”, and variations such as “comprises”, “comprising” and “possesses”, will be understood to imply the inclusion of a stated integer or step or group of integers or steps but not the exclusion of any other integer or step or group of integer or step. The term “comprising” also means “including” as well as “consisting” e.g. a composition “comprising” X may consist exclusively of X or may include something additional e.g. X+Y. It must be noted also that, as used in this specification and the appended claims, the singular forms “a”, “an” and “the” include plural referents unless the content clearly dictates otherwise. By way of example, a reference to “a gene” or “an enzyme” is a reference to “one or more genes” or “one or more enzymes”.

It is to be understood that this disclosure is not limited to the particular methodology, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by the person skilled in the art. In accordance with the present disclosure there may be conventional molecular biology, microbiology, and recombinant DNA techniques employed which are within the skill of the art.

This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. Preferably, the terms used herein are defined as described in “A multilingual glossary of biotechnological terms: (IUPAC Recommendations)”, Leuenberger, H. G. W, Nagel, B. and Kolbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety.

The examples and figures which follow are presented by way of illustration and without implied limitation of the invention.

### EXAMPLES

### Example 1: Protocol for Making a Recombinant Saccharomyces cerevisiae Strain According to the Invention

All the hereinafter implemented recombinant Saccharomyces cerevisiae strains were constructed from standard strains using standard yeast molecular genetics procedure (Methods in yeast Genetics-A cold spring harbor laboratory course Manual (2000) by D. Burke, D. Dawson, T. Stearns CSHL Press).

Cluster of the following-mentioned genes were integrated in recombinant yeast at once using the ability of yeast to efficiently recombine free DNA ends which have sequence homology.

In addition, for a better comprehension of following genotypes:
jlp1, lyp1, sam3, his3, leu2, trp1 and ura3 are insertion sites.
Lowercase letters mean that the considered gene is inactive, uppercase letters reflect an active gene.
“::”: following a gene name means that the gene is interrupted by what follows (if more than one gene are inserted, they are noted in brackets [ ]). The interruption of the gene is concomitant with an entire deletion of the coding sequence but preserves the promoter. In consequence the gene followed by “::” is inactive and is noted in lowercase. If not specified, the transcription of the gene inserted is controlled by the promoter of the disrupted gene.
“gene.Kl” means that the gene originates from Kluyveromyces lactis. When nothing is indicated after a gene in the present examples, this means that the gene is from Saccharomyces cerevisiae.

More particularly, the coding sequences to be cloned were artificially synthetized. For heterologous sequences (non-yeast), the nucleic sequences were modified in order to obtain a synonymous coding sequence using the yeast codon usage. Using restriction enzyme and classical cloning technology, each synthetic sequence was cloned in between a transcription promoter and a transcription terminator. Each promoter sequence is preceded by a 50 to 200 nucleotide sequence homologous to the sequence of the terminator of the upstream gene. Similarly, the terminator of each gene (a gene comprising the promoter-coding sequence-terminator) is followed by sequences homologous to the gene immediately following. So that each of the unit to be integrated have a 50-200 nucleotide overlap with both the unit upstream and the unit downstream. For the first unit, the promoter is preceded by 50-200 nucleotides homologous to the yeast chromosome nucleotide for the locus in which it will be integrated. Similarly, for the last unit, the terminator is followed by 50-200 nucleotides homologous to the yeast chromosome nucleotide for the locus in which it will be integrated.

Each unit is then PCR amplified from the plasmids constructs, yielding X unit of linear DNA having overlapping sequences. At least one of this gene is an auxotrophic marker, in order to select for recombination event. All the linear fragments are transformed in the yeast at once, and a recombinant yeast cell is selected for the auxotrophy related to the marker used. The integrity of the sequence is then verified by PCR and sequencing.

### Example 2: Comparative Examples for the Production of Heparosan

Seven recombinant strains are derived from the Wild Type strain CC788-2D (Cherest et al. (2000) J Biol. Chem. 275:14056-14063) which does not produce any heparosan: YA5398-3, YA5402-1, YA5400-1, YA5401-1, YA5437-18, YA5435-2 and DA2531-12.

These seven strains are as follows:
YA5398-3: MAT-α, can1-100, his3: [tRPL3-UGP1-pSAM1, pMET6-QRI1-tIDP1, HIS3] x8, leu2::[URA3.Sba-loxP, pCCW12.Sba-HASB.Vir-tRPL3.Sm, pCCW12.Sk-HASB-A. Vir-tTEF1.Sba, pCCW12.Sar-HSS2.Pm-tRPL3.Sba, pCCW120.Sca-HSS2.Pm-tRPL15A.Sm], leu2, trp1, ura3
YA5402-1: MAT-α, can1-100, his3::[tRPL3-UGP1-pSAM1, pMET6-QRI1-tIDP1, HIS3] x8, jlp1::[LEU2.Sba-loxP, pCCW12.Sba-HASB.Vir-tRPL3.Sm, pCCW12.Sk-HASB-A. Vir-tTEF1.Sba, pCCW12.Sar-HSS2.Pm-tRPL3.Sba, pCCW120.Sca-HSS2.Pm-tRPL15A.Sm], leu2, trp1, ura3
YA5400-1: MAT-α, can1-100, his3::[tRPL3-UGP1-pSAM1, pMET6-QRI1-tIDP1, HIS3] x8, jlp1::[LEU2.Sba-loxP, pCCW12.Sba-HASB.Vir-tRPL3.Sm, pCCW12.Sk-HASB-A. Vir-tTEF1.Sba, pCCW12.Sar-HSS2.Pm-tRPL3.Sba, pCCW120.Sca-HSS2.Pm-tRPL15A.Sm, pTEF1.Ago-GFA1-tRPL15A], leu2, trp1, ura3
YA5401-1: MAT-α, can1-100, his3: [tRPL3-UGP1-pSAM1, pMET6-QRI1-tIDP1, HIS3] x8, jlp1::[LEU2.Sba-loxP, pCCW12.Sba-HASB.Vir-tRPL3.Sm, pCCW12.Sk-HASB-A. Vir-tTEF1.Sba, pCCW12.Sar-HSS2.Pm-tRPL3.Sba, pCCW120.Sca-HSS2.Pm-[RPL15A.Sm, pTEF1.Ago-GFA1.Vir-tRPL15A], leu2, trp1, ura3
YA5437-18: MAT-α, can1-100, his3::[tRPL3-UGP1-pSAM1, pMET6-QRI1-IDP1, HIS3] x8, jlp1::[LEU2.Sba-loxP, pCCW12.Sba-HASB.Vir-tRPL3.Sm, pCCW12.Sk-HASB-A. Vir-tTEF1.Sba, pCCW12.Sar-HSS1.Pm-tRPL15A.Sm], leu2, trp1, ura3
YA5435-2: MAT-α, can1-100, his3::[tRPL3-UGP1-pSAM1, pMET6-QRI1-tDP1, HIS3] x8, jlp1::[LEU2.Sba-loxP, pCCW12.Sba-HASB.Vir-tRPL3.Sm, pCCW12.Sk-HASB-A. Vir-tTEF1.Sba, pCCW12.Sar-HSS1.Pm-tRPL15A.Sm, pTEF1.Ago-GFA1.Vir-tRPL15A], leu2, trp1, ura3
DA2531-12: MAT-α/MAT-α, CANI/can1-100, his3/his3::[tRPL3-UGP1-pSAM1, pMET6-QRI1-tIDP1, HIS3] x8, jlp1::[LEU2.KI-RS, pCUP1-UGP1-tRPL3, pCUP1-QRI1-tIDP1, pPDC1-UGP1-tTPI1, pTDH3-QRI1-tMET25, pCCW12-HASB.At-RPL15A]/jlp1::[LEU2.Sba-loxP, pCCW12.Sba-HASB.Vir-tRPL3.Sm, pCCW12.Sk-HASB-A. Vir-tTEF1.Sba, pCCW12.Sar-HSS1.Pm-tRPL15A.Sm, pTEF1.Ago-GFA1. Vir-tRPL15A], leu2/leu2, SAM3/sam3::[LEU2.KI-RS, pPDC1-PGM1-tIDP1, pTEF1.Ago-GFA1-tRPL15A, pENO2-UGP1-tRPL3, pCWP2-GNA1-tTPI1, pTEF1-PCM1-tRPL41B, pCCW12.Sba-HASB.vir-tRPL3.Sm, pCCW12.Sk-HASB-A. Vir-tTEF1.Sba, pCCW12.Sar-HSS2.Pm-tRPL3.Sba, pCCW120.Sca-HSS2.Pm-tRPL15A.Sm, pTDH3-1.Sba-QRI1-tIDP1.Sba, HIS3.Sba-loxP], trp1/trp1, ura3
HSS1.Pm represents a sequence encoding an heparosan synthase A from Pasteurella multocida (UniProt reference Q8RP78). HSS2.Pm represents a sequence encoding an heparosan synthase B from Pasteurella multocida (UniProt reference Q5SGE1).

All these strains were incubated in 25 ml of SY medium buffered with MES 0.1 M ph 5.5 baffled erlenmeyer for 48 hours at 28° C. under vigourous agitation in Erlenmeyers flasks.

SY medium comprises the following elements:

KH2PO4: 100 mM; MgSO4 7H2O: 2.8 mM; K2SO4: 11.5 mM; Na2SO4: 1.1 mM; NaCl: 2.6 mM; CaCl2 2H2O: 0.7 mM; CuSO4 5H2O: 15 μM; KI: 6 μM; FeC13: 30 μM; ZnSO4 7H2O: 61 μM; MnSO4 H2O: 25 μM; H2SO4: 110 μM; Panthotenic Acids hemicalcium salt: 42 μM; Thiamin hydrochloride: 59 μM; Pyridoxin hydrochloride: 49 μM; Myo-Inositol (C6H1206): 555 μM; Nicotinic acid (C6H5NO2): 29 UM; D-Biotine: 0.82 μM; Ammonium citrate tribasic: 33 mM; and glucose 2%

Growth medium was recovered at 48h hours and assayed for heparosan content and quality. An aliquot of the medium was loaded and ran on a 0.5% agarose gel subsequently colored with bromophenol blue. The amount of heparosan present in the medium was evaluated by a colorimetric determination after treatment with concentrated sulfuric acid and carbazole (Bitter and Muir (1962) analytical biochemistry 4, 330-334).

The amounts of heparosan obtained with these different strains are respectively:
YA5398-3:170 mg·L⁻¹.
YA5402-1:170 mg·L⁻¹.
YA5400-1:110 mg·L⁻¹.
YA5401-1:180 mg·L⁻¹.
YA5437-18:200 mg·L⁻¹.
YA5435-2:190 mg·L⁻¹.
DA2531-12:235 mg·L⁻¹.

In comparison, as indicated above, a native strain does not produce heparosan.

It results from this experiment that a recombinant strain comprising the modifications according to the invention produces a greater amount of heparosan when cultured in the same conditions as compared with other recombinant strains not comprising all the genetic modifications according to the invention.

SEQUENCES

SEQ ID NO: 1 is a reencoded nucleic acid sequence of heparosan synthase
originating from Pasteurella multocida (HSS1)
ATGAGTTTGTTCAAAAGGGCGACTGAGCTGTTCAAATCGGGTAACTATAAGGATGCACTGACATTGTACGAGAAC
ATAGCCAAAATTTATGGCTCAGAATCCCTAGTGAAGTATAACATAGATATCTGCAAGAAGAACATAACCCAATCA
AAATCGAACAAGATAGAAGAGGACAATATTTCCGGAGAGAATAAGTTCTCCGTCTCTATCAAAGACTTATACAAC
GAAATTAGTAATTCTGAGCTAGGGATAACAAAGGAGAGATTAGGAGCACCACCCTTGGTAAGCATAATTATGACG
AGTCATAATACAGAGAAATTCATCGAAGCTTCCATCAACAGTTTATTATTGCAAACTTATAACAATCTTGAGGTC
ATTGTGGTAGACGACTACTCCACAGACAAGACCTTCCAAATCGCGTCCAGGATTGCAAACTCGACGTCAAAAGTT
AAGACTTTCAGATTGAATTCCAACCTTGGAACGTACTTCGCAAAGAATACGGGAATCCTAAAATCAAAAGGGGAC
ATTATATTCTTTCAAGATTCTGATGATGTCTGCCATCATGAACGTATTGAGAGATGTGTAAATGCACTTTTGTCT
AACAAAGATAACATTGCCGTTAGGTGCGCTTATTCTCGTATTAATCTGGAGACCCAGAACATCATAAAGGTCAAC
GACAACAAGTACAAGTTAGGTCTTATTACCCTAGGAGTATACCGTAAAGTATTTAATGAAATAGGATTCTTCAAC
TGTACCACTAAGGCAAGTGACGACGAATTTTATCACAGGATCATCAAATACTATGGCAAGAATAGGATAAACAAT
TTGTTCCTACCTCTATACTACAATACCATGCGTGAAGATAGTTTGTTTAGCGATATGGTCGAGTGGGTAGACGAA
AACAACATTAAACAGAAAACAAGTGATGCACGTCAGAATTATCTGCATGAATTCCAAAAGATCCACAACGAAAGG
AAATTGAATGAGTTAAAGGAGATTTTCAGCTTCCCCAGGATTCACGATGCCCTACCGATAAGTAAAGAGATGAGC
AAGTTGTCTAACCCAAAGATCCCTGTCTACATTAACATTTGCAGTATACCATCTAGGATAAAGCAATTACAGTAT
ACGATCGGAGTGCTGAAGAATCAGTGTGACCATTTCCACATTTACTTAGATGGTTATCCAGAGGTTCCAGATTTC
ATAAAGAAGCTAGGCAACAAGGCTACCGTAATCAATTGCCAAAACAAGAACGAGTCTATCAGGGACAACGGCAAG
TTTATCCTTTTAGAAAAGCTGATCAAAGAGAATAAGGACGGCTATTATATTACCTGTGATGATGACATAAGGTAT
CCTGCTGACTACACGAATACCATGATAAAGAAGATTAACAAGTACAATGACAAAGCGGCAATAGGTCTACACGGC
GTCATCTTTCCGTCCAGAGTGAATAAATACTTCTCATCTGACAGGATTGTTTATAACTTTCAGAAGCCCTTGGAA
AACGACACAGCCGTCAACATTTTGGGTACTGGGACCGTTGCATTTAGAGTCTCCATCTTCAACAAGTTTTCCCTG
AGCGACTTCGAGCACCCTGGTATGGTGGATATTTATTTCTCAATACTATGCAAGAAGAACAACATATTGCAAGTT
TGTATAAGCAGGCCCTCAAACTGGTTAACCGAGGACAACAAGAATACCGAAACCCTGTTCCACGAGTTCCAAAAC
CGTGACGAGATCCAGAGTAAGTTGATCATCTCTAATAATCCGTGGGGCTACAGTAGCATCTACCCCTTACTTAAC
AACAATGCCAACTATAGCGAGTTGATACCATGCTTATCATTCTATAATGAGTAA
SEQ ID NO: 2 is a reencoded nucleic acid sequence of heparosan synthase
originating from Pasteurella multocida (HSS2)
ATGAAGGGCAAGAAAGAGATGACCCAAATACAGATTGCTAAGAACCCGCCCCAACACGAGAAGGAAAACGAGCTG
AATACTTTCCAAAACAAGATCGACTCCCTTAAAACGACATTAAACAAGGACATTATATCACAACAGACATTGTTG
GCTAAACAGGACTCAAAGCACCCATTATCTGCAAGTCTTGAGAACGAGAACAAACTTCTTCTTAAACAGTTGCAG
CTTGTTTTGCAGGAATTTGAAAAGATATACACTTACAACCAAGCACTAGAGGCTAAGCTTGAGAAAGACAAACAG
ACCACCTCAATAACCGATTTATATAACGAAGTTGCAAAGTCTGATCTAGGGCTAGTTAAGGAAACTAACAGTGCT
AACCCTCTAGTCTCCATTATTATGACTTCGCACAATACCGCTCAATTTATCGAGGCATCGATTAACTCCTTGTTG
CTACAGACATATAAGAACATTGAGATAATTATAGTCGATGATGACAGTTCCGACAACACTTTTGAAATCGCCTCA
AGGATCGCAAACACAACGTCTAAGGTCCGTGTGTTTCGTTTGAACTCCAATCTGGGCACGTACTTTGCTAAGAAT
ACAGGAATCCTTAAGAGTAAAGGCGACATAATATTCTTCCAGGACTCCGACGACGTTTGTCATCATGAGAGAATC
GAAAGGTGCGTGAACATTTTGTTGGCTAACAAAGAGACGATTGCTGTGAGGTGCGCATACAGTAGGCTGGCTCCT
GAAACGCAGCATATAATTAAAGTCAACAACATGGATTATAGGCTTGGTTTCATCACACTGGGAATGCATAGGAAG
GTTTTCCAAGAGATTGGGTTCTTCAACTGTACAACAAAAGGCAGTGACGACGAGTTCTTCCACCGTATAGCTAAA
TACTACGGTAAAGAAAAGATTAAGAACCTTTTATTGCCGCTTTATTATAACACGATGCGTGAAAACTCACTTTTC
ACTGACATGGTCGAGTGGATCGACAACCATAATATCATACAGAAGATGTCTGACACACGTCAACACTATGCTACT
CTGTTTCAGGCGATGCATAATGAGACTGCATCACACGACTTCAAGAACTTGTTCCAGTTCCCACGTATATACGAC
GCCTTGCCTGTTCCACAGGAGATGTCTAAGTTATCTAACCCTAAAATACCGGTGTACATAAATATATGCAGTATA
CCATCTAGGATTGCACAGTTACGTAGGATTATTGGGATCTTAAAGAACCAGTGCGACCACTTTCACATATACCTA
GACGGATACGTGGAGATCCCTGATTTTATTAAGAATCTTGGTAATAAAGCAACGGTCGTCCACTGCAAAGACAAG
GACAACTCTATTAGGGACAACGGCAAGTTTATTCTACTGGAAGAGTTAATAGAAAAGAATCAAGACGGGTACTAC
ATAACGTGCGACGACGACATTATCTATCCATCGGACTATATAAACACAATGATTAAGAAGCTGAACGAGTATGAT
GACAAGGCGGTCATAGGGCTTCACGGAATATTATTTCCGTCAAGGATGACGAAATACTTTTCCGCCGATAGGCTT
GTTTATTCGTTTTACAAACCCCTAGAGAAAGACAAAGCAGTAAATGTTCTTGGGACCGGAACTGTCTCATTCAGA
GTTAGTCTGTTCAACCAATTCTCCCTTTCAGACTTCACCCATTCGGGAATGGCCGACATCTACTTCTCTCTATTG
TGCAAGAAGAACAACATTTTACAAATCTGTATCTCCAGACCGGCAAATTGGTTGACGGAAGATAACAGAGATTCT
GAGACCTTATACCACCAGTATAGAGATAATGACGAGCAGCAGACTCAGTTGATCATGGAAAATGGGCCCTGGGGT
TACTCAAGTATCTATCCACTTGTCAAGAATCACCCGAAGTTTACGGACTTAATACCATGTTTGCCTTTCTACTTC
CTATAA
SEQ ID NO: 3 is an amino acid sequence of heparosan synthase originating
from Pasteurella multocida (HSS1)
MSLFKRATELFKSGNYKDALTLYENIAKIYGSESLVKYNIDICKKNITQSKSNKIEEDNISGENKFSVSIKDLYN
EISNSELGITKERLGAPPLVSIIMTSHNTEKFIEASINSLLLQTYNNLEVIVVDDYSTDKTFQIASRIANSTSKV
KTFRLNSNLGTYFAKNTGILKSKGDIIFFQDSDDVCHHERIERCVNALLSNKDNIAVRCAYSRINLETQNIIKVN
DNKYKLGLITLGVYRKVFNEIGFFNCTTKASDDEFYHRIIKYYGKNRINNLFLPLYYNTMREDSLFSDMVEWVDE
NNIKQKTSDARQNYLHEFQKIHNERKLNELKEIFSFPRIHDALPISKEMSKLSNPKIPVYINICSIPSRIKQLQY
TIGVLKNQCDHFHIYLDGYPEVPDFIKKLGNKATVINCONKNESIRDNGKFILLEKLIKENKDGYYITCDDDIRY
PADYTNTMIKKINKYNDKAAIGLHGVIFPSRVNKYFSSDRIVYNFQKPLENDTAVNILGTGTVAFRVSIFNKFSL
SDFEHPGMVDIYFSILCKKNNILQVCISRPSNWLTEDNKNTETLFHEFQNRDEIQSKLIISNNPWGYSSIYPLLN
NNANYSELIPCLSFYNE
SEQ ID NO: 4 is an amino acid sequence of heparosan synthase originating
from Pasteurella multocida (HSS2)
MAKGKKEMTQIQIAKNPPQHEKENELNTFQNKIDSLKTTLNKDI ISQQTLLAKQDSKHPLSASL
ENENKLLLKQLQLVLQEFEKTYTYNQALEAKLEKDKQTTSITDLYNEVAKSDLGLVKETNSANPLVSI IMTSHNT
AQFIEASINSLLLQTYKNIEIIIVDDDSSDNTFEIASRIANTTSKVRVFRLNSNLGTYFAKNTGILKSKGDIIFF
QDSDDVCHHERIERCVNILLANKETIAVRCAYSRLAPETQHIIKVNNMDYRLGFITLGMHRKVFQEIGFFNCTTK
GSDDEFFHRIAKYYGKEKIKNLLLPLYYNTMRENSLFTDMVEWIDNHNIIQKMSDTRQHYATLFQAMHNETASHD
FKNLFQFPRIYDALPVPQEMSKLSNPKIPVYINICSIPSRIAQLRRIIGILKNQCDHFHIYLDGYVEIPDFIKNL
GNKATVVHCKDKDNSIRDNGKFILLEELIEKNQDGYYITCDDDIIYPSDYINTMIKKLNEYDDKAVIGLHGILFP
SRMTKYFSADRLVYSFYKPLEKDKAVNVLGTGTVSFRVSLFNQFSLSDFTHSGMADIYFSLLCKKNNILQICISR
PANWLTEDNRDSETLYHQYRDNDEQQTQLIMENGPWGYSSIYPLVKNHPKFTDLIPCLPFYFL
SEQ ID NO: 5 is a nucleic acid sequence of UDP-Glucose dehydrogenase
(HASB) originating from Arabidopsis thaliana
ATGGTCAAAATATGCTGTATCGGGGCTGGATATGTCGGTGGGCCTACAATGGCCGTTATGGCCCTAAAGTGTCCA
GAGATTGAAGTCGTGGTAGTTGATATATCGGAGCCACGAATTAACGCATGGAACTCAGACCGTCTACCAATTTAT
GAGCCAGGGTTAGAGGATGTCGTCAAACAGTGTAGAGGGAAGAATTTGTTCTTCTCTACAGATGTAGAGAAGCAT
GTATTTGAGTCAGATATAGTGTTTGTTTCGGTAAACACTCCTACGAAAACGCAGGGTCTGGGTGCAGGTAAAGCA
GCAGATTTGACATATTGGGAGTCCGCTGCTCGCATGATAGCTGATGTGAGCAAATCGTCGAAAATCGTCGTTGAA
AAGAGTACAGTACCAGTTCGTACAGCCGAGGCTATAGAAAAGATTTTAACGCACAATTCGAAGGGTATCGAATTC
CAGATCTTATCAAATCCAGAATTCTTGGCCGAAGGGACGGCGATTAAAGACTTATATAACCCTGATAGAGTTCTA
ATCGGTGGCAGGGACACCGCTGCGGGACAAAAGGCCATTAAGGCGTTGCGTGACGTGTATGCCCACTGGGTTCCT
GTTGAGCAAATAATCTGTACTAATTTATGGAGTGCCGAGCTATCAAAGTTGGCTGCGAACGCATTTCTAGCTCAA
AGGATAAGTTCAGTAAACGCAATGTCAGCGCTTTGTGAGGCAACTGGTGCTGACGTAACCCAAGTCGCTCACGCC
GTTGGAACCGACACTAGAATTGGACCGAAGTTTCTTAACGCATCCGTAGGCTTCGGCGGATCTTGCTTTCAGAAA
GACATCCTGAATCTTATTTACATCTGCGAATGCAATGGTCTTCCAGAAGCAGCCAATTATTGGAAACAGGTAGTC
AAGGTAAATGACTACCAAAAGATTAGGTTTGCTAATCGAGTCGTATCTTCTATGTTCAACACCGTCTCCGGTAAG
AAAATTGCTATTTTGGGATTTGCGTTCAAGAAGGACACCGGCGACACGCGTGAAACTCCTGCCATAGATGTGTGT
AATCGCCTCGTGGCTGATAAAGCAAAGCTGTCGATCTATGATCCGCAAGTATTAGAAGAGCAGATCCGCCGTGAT
CTGTCCATGGCCCGATTCGATTGGGACCATCCAGTCCCACTCCAGCAGATCAAAGCTGAAGGTATCTCCGAACAG
GTTAACGTTGTGTCCGACGCCTACGAGGCTACGAAGGATGCTCATGGTTTATGTGTTTTAACCGAATGGGACGAA
TTCAAGTCACTTGATTTTAAGAAGATCTTTGATAATATGCAGAAACCCGCTTTCGTTTTCGACGGAAGAAACGTG
GTCGACGCTGTGAAATTGAGAGAAATTGGATTCATAGTATATTCCATAGGTAAACCTCTGGATAGTTGGCTCAAG
GATATGCCGGCTGTTGCATAA
SEQ ID NO: 6 is a reencoded nucleic acid sequence of UDP-Glucose
dehydrogenase (HASB) originating from Chlorella virus PBCV-1
ATGAGTAGAATTGCTGTCGTTGGATGCGGTTACGTGGGTACGGCCTGCGCCGTACTTTTGGCGCAGAAGAACGAA
GTTATCGTTTTGGATATCTCGGAGGACCGGGTACAACTGATTAAGAATAAGAAGTCACCTATAGAAGATAAGGAA
ATCGAAGAATTCCTGGAGACGAAAGATTTGAATCTAACAGCGACGACGGATAAGGTGCTCGCCTATGAGAATGCT
GAATTCGTTATAATAGCTACACCGACCGATTACGATGTCGTCACTAGATATTTCAACACTAAGTCTGTTGAAAAT
GTGATAGGCGATGTCATTAAGAACACTCAGACACACCCTACTATAGTGATCAAGAGTACTATTCCAATCGGTTTC
GTGGATAAAGTTAGAGAACAGTTCGATTATCAAAATATTATCTTCTCGCCGGAATTCTTGAGAGAAGGACGAGCA
TTATATGATAATCTTTACCCCTCCCGTATCATCGTCGGTGATGACTCCCCAATTGCCTTAAAATTCGCGAATCTC
TTGGTCGAGGGTAGCAAAACTCCACTAGCTCCCGTATTAACTATGGGTACGCGAGAGGCCGAAGCTGTAAAACTA
TTTTCAAATACATATTTGGCTATGAGAGTAGCATACTTCAATGAGCTAGACACATTTGCAATGTCCCATGGTATG
AACGCAAAAGAGATTATCGACGGAGTCACACTAGAACCAAGGATAGGGCAGGGTTATTCGAATCCTTCATTTGGG
TATGGAGCCTATTGTTTCCCAAAAGATACAAAGCAATTGTTGGCTAATTTTGAAGGGGTTCCACAAGATATTATT
GGCGCAATCGTAGAGTCTAACGAAACTAGAAAGGAGGTTATTGTTTCTGAGGTCGAGAACAGATTTCCGACCACA
GTTGGCGTATATAAGTTGGCTGCTAAAGCTGGTTCGGATAATTTCAGGTCTAGTGCGATAGTCGATATCATGGAA
AGGTTGGCTAATAAGGGCTACCACATCAAAATATTTGAACCTACCGTTGAACAATTTGAGAATTTTGAGGTAGAT
AATAATCTCACAACTTTTGCAACCGAGTCTGATGTAATAATTGCGAACAGAGTTCCAGTCGAACATCGCATTCTG
TTTGGGAAGAAGTTAATAACACGGGATGTCTATGGGGATAA
SEQ ID NO: 7 is a reencoded nucleic acid sequence of UDP-Glucose
dehydrogenase (HASB-A) originating from Chlorella virus PBCV-1
ATGTCACGAATCGCAGTAGTTGGCTGTGGGTACGTGGGAACCGCATGCGCTGTACTGCTGGCGCAAAAGAATGAG
GTAATCGTTCTTGATATTAGTGAGGATAGGGTCCAACTAATCAAGAACAAGAAATCCCCGATCGAAGATAAGGAG
ATTGAAGAATTCTTGGAAACAAAAGACCTAAACTTAACTGCAACAACTGACAAAGTTTTAGCCTACGAAAACGCT
GAGTTTGTGATTATAGCGACACCCACAGATTATGACGTAGTTACCAGATACTTTAACACGAAGTCCGTAGAGAAC
GTCATTGGAGATGTTATAAAGAATACTCAGACTCATCCTACGATAGTAATAAAGTCAACCATTCCCATAGGTTTC
GTAGATAAGGTTAGGGAGCAATTCGATTACCAGAACATTATATTTTCGCCAGAATTTCTGAGAGAGGGTCGCGCC
CTGTATGATAATCTATATCCATCACGGATTATAGTGGGCGATGACTCTCCGATTGCACTTAAGTTTGCTAATCTT
TTAGTTGAGGGCTCCAAAACTCCGCTCGCCCCAGTACTTACGATGGGTACACGTGAGGCTGAAGCTGTCAAGCTG
TTTTCAAACACATACCTTGCTATGCGAGTCGCATACTTTAACGAACTAGATACCTTTGCTATGTCGCACGGTATG
AATGCTAAAGAAATCATAGATGGCGTAACGTTGGAGCCTCGGATAGGTCAAGGATATTCCAATCCATCTTTTGGC
TACGGTGCGTATTGTTTCCCAAAGGACACGAAGCAATTATTAGCTAACTTTGAGGGTGTTCCGCAAGATATAATT
GGGGCGATAGTAGAAAGCAATGAAACACGGAAGGAGGTAATCGTGAGTGAAGTGGAAAACCGATTCCCCACTACG
GTCGGCGTTTACAAATTAGCCGCCAAGGCTGGTTCCGACAATTTCCGATCCAGCGCAATAGTAGATATTATGGAA
AGATTAGCTAATAAGGGATACCACATTAAAATCTTTGAACCTACTGTCGAACAGTTCGAGAACTTCGAGGTTGAT
AATAACTTGACGACTTTCGCAACGGAGAGCGATGTAATTATTGCAAACCGCGTACCTGTGGAACATCGAATTTTG
TTCGGAAAGAAGCTGATTACACGCGATGTATATGGCGATAACTAA
SEQ ID NO: 8 is an amino acid sequence of UDP-Glucose dehydrogenase
(HASB) originating from Arabidopsis thaliana
MVKICCIGAGYVGGPTMAVMALKCPEIEVVVVDISEPRINAWNSDRLPIYEPGLEDVVKQCRGKNLFFSTDVEKH
VFESDIVFVSVNTPTKTQGLGAGKAADLTYWESAARMIADVSKSSKIVVEKSTVPVRTAEAIEKILTHNSKGIEF
QILSNPEFLAEGTAIKDLYNPDRVLIGGRDTAAGQKAIKALRDVYAHWVPVEQIICTNLWSAELSKLAANAFLAQ
RISSVNAMSALCEATGADVTQVAHAVGTDTRIGPKFLNASVGFGGSCFQKDILNLIYICECNGLPEAANYWKQVV
KVNDYQKIRFANRVVSSMFNTVSGKKIAILGFAFKKDTGDTRETPAIDVCNRLVADKAKLSIYDPQVLEEQIRRD
LSMARFDWDHPVPLQQIKAEGISEQVNVVSDAYEATKDAHGLCVLTEWDEFKSLDFKKIFDNMQKPAFVFDGRNV
VDAVKLREIGFIVYSIGKPLDSWLKDMPAVA
SEQ ID NO: 9 is an amino acid sequence of UDP-Glucose dehydrogenase
(HASB) originating from Chlorella virus PBCV1
MSRIAVVGCGYVGTACAVLLAQKNEVIVLDISEDRVQLIKNKKSPIEDKEIEEFLETKDLNLTATTDKVLAYENA
EFVIIATPTDYDVVTRYFNTKSVENVIGDVIKNTQTHPTIVIKSTIPIGFVDKVREQFDYQNIIFSPEFLREGRA
LYDNLYPSRIIVGDDSPIALKFANLLVEGSKTPLAPVLTMGTREAEAVKLFSNTYLAMRVAYFNELDTFAMSHGM
NAKEIIDGVTLEPRIGQGYSNPSFGYGAYCFPKDTKQLLANFEGVPQDIIGAIVESNETRKEVIVSEVENRFPTT
VGVYKLAAKAGSDNFRSSAIVDIMERLANKGYHIKIFEPTVEQFENFEVDNNLTTFATESDVIIANRVPVEHRIL
FGKKLITRDVYGDN
SEQ ID NO: 10 is a reencoded nucleic acid sequence of glucuronosyl-
disulfoglucosamine glucuronidase originating from Pedobacter heparinus with a N-terminal
secretion signal
ATGCAATTCTCGACGGTCGCAAGTGTTGCCTTTGTTGCTTTGGCTAACTTCGTTGCCGCACAGTCCTCGTCAATC
ACCAGGAAGGACTTCGATCATATCAACTTGGAATACTCCGGTTTGGAGAAGGTGAACAAGGCGGTAGCAGCAGGC
AATTACGACGACGCGGCAAAAGCCCTACTAGCTTACTACCGTGAAAAGAGCAAAGCAAGAGAGCCTGACTTTAGT
AATGCCGAGAAGCCTGCCGACATACGTCAGCCTATTGACAAGGTAACACGTGAGATGGCGGATAAAGCATTAGTA
CATCAGTTTCAACCTCACAAAGGATACGGATATTTTGATTATGGGAAGGATATTAATTGGCAGATGTGGCCAGTT
AAAGACAACGAGGTCAGATGGCAGTTGCATAGGGTAAAATGGTGGCAGGCAATGGCCTTGGTTTACCATGCCACG
GGCGACGAGAAGTACGCCAGGGAGTGGGTGTACCAATACTCGGACTGGGCGAGGAAGAACCCGTTAGGCTTATCG
CAAGACAATGATAAGTTTGTCTGGAGACCTTTGGAAGTCTCAGATAGAGTCCAGTCATTGCCACCTACCTTTTCT
CTTTTCGTCAACAGTCCCGCCTTCACACCAGCATTCCTAATGGAGTTCTTAAACTCGTACCACCAACAGGCAGAC
TACTTGTCAACACACTACGCCGAGCAGGGGAACCATAGGCTGTTCGAAGCTCAGAGGAACTTGTTCGCCGGCGTA
TCCTTCCCCGAATTCAAGGACTCACCCAGGTGGAGACAGACTGGAATATCTGTTCTGAATACAGAGATAAAGAAG
CAGGTGTACGCCGATGGCATGCAATTCGAGTTAAGTCCGATATACCACGTTGCTGCCATAGATATCTTTCTTAAA
GCATACGGAAGCGCCAAGAGAGTTAATTTGGAGAAAGAGTTCCCTCAATCCTATGTGCAAACCGTCGAGAATATG
ATTATGGCGTTAATTTCAATAAGTCTACCGGACTATAATACGCCCATGTTCGGCGACTCTTGGATCACTGATAAG
AATTTCAGAATGGCTCAATTTGCCTCATGGGCGCGTGTCTTCCCCGCTAACCAGGCAATTAAGTACTTTGCAACC
GACGGAAAACAGGGTAAAGCTCCCAACTTCTTGAGTAAGGCATTGTCCAATGCAGGATTCTACACGTTTAGAAGT
GGATGGGATAAGAACGCGACAGTGATGGTACTGAAGGCAAGTCCGCCTGGTGAGTTTCACGCTCAGCCTGATAAC
GGAACTTTTGAACTGTTCATCAAGGGTAGGAACTTCACACCCGACGCCGGTGTATTCGTCTATTCCGGTGACGAG
GCGATTATGAAATTGAGGAACTGGTACAGGCAGACAAGGATTCACTCTACTCTAACTCTTGATAATCAGAATATG
GTCATAACGAAAGCACGTCAGAATAAATGGGAGACAGGCAATAACTTGGATGTCTTAACTTACACTAACCCGTCA
TACCCCAACTTAGATCACCAACGTTCAGTTCTATTCATTAACAAGAAGTATTTCTTAGTCATAGATAGGGCTATC
GGCGAGGCCACGGGCAACCTTGGCGTGCACTGGCAGCTAAAGGAAGACAGTAACCCTGTCTTCGATAAAACCAAG
AATAGAGTCTATACTACCTATAGGGACGGGAACAATCTAATGATACAGAGTCTAAACGCAGACAGGACATCACTG
AACGAGGAAGAAGGGAAGGTATCTTATGTCTATAACAAGGAGCTGAAGCGTCCAGCTTTCGTATTTGAGAAGCCT
AAGAAGAACGCAGGGACCCAAAACTTTGTGTCAATTGTCTATCCATACGACGGTCAGAAGGCGCCGGAAATTTCT
ATTAGAGAAAATAAGGGAAATGACTTCGAAAAGGGAAAATTGAACCTAACACTGACTATAAACGGTAAGCAACAG
CTTGTGTTGGTTCCTTAA
SEQ ID NO: 11 is a reencoded nucleic acid sequence of glucuronosyl-
disulfoglucosamine glucuronidase originating from Pedobacter heparinus with a N-terminal
secretion signal and a C-terminal anchoring signal
ATGCAATTCTCGACGGTCGCAAGTGTTGCCTTTGTTGCTTTGGCTAACTTCGTTGCCGCACAGTCCTCGTCAATC
ACCAGGAAGGACTTCGATCATATCAACTTGGAATACTCCGGTTTGGAGAAGGTGAACAAGGCGGTAGCAGCAGGC
AATTACGACGACGCGGCAAAAGCCCTACTAGCTTACTACCGTGAAAAGAGCAAAGCAAGAGAGCCTGACTTTAGT
AATGCCGAGAAGCCTGCCGACATACGTCAGCCTATTGACAAGGTAACACGTGAGATGGCGGATAAAGCATTAGTA
CATCAGTTTCAACCTCACAAAGGATACGGATATTTTGATTATGGGAAGGATATTAATTGGCAGATGTGGCCAGTT
AAAGACAACGAGGTCAGATGGCAGTTGCATAGGGTAAAATGGTGGCAGGCAATGGCCTTGGTTTACCATGCCACG
GGCGACGAGAAGTACGCCAGGGAGTGGGTGTACCAATACTCGGACTGGGCGAGGAAGAACCCGTTAGGCTTATCG
CAAGACAATGATAAGTTTGTCTGGAGACCTTTGGAAGTCTCAGATAGAGTCCAGTCATTGCCACCTACCTTTTCT
CTTTTCGTCAACAGTCCCGCCTTCACACCAGCATTCCTAATGGAGTTCTTAAACTCGTACCACCAACAGGCAGAC
TACTTGTCAACACACTACGCCGAGCAGGGGAACCATAGGCTGTTCGAAGCTCAGAGGAACTTGTTCGCCGGCGTA
TCCTTCCCCGAATTCAAGGACTCACCCAGGTGGAGACAGACTGGAATATCTGTTCTGAATACAGAGATAAAGAAG
CAGGTGTACGCCGATGGCATGCAATTCGAGTTAAGTCCGATATACCACGTTGCTGCCATAGATATCTTTCTTAAA
GCATACGGAAGCGCCAAGAGAGTTAATTTGGAGAAAGAGTTCCCTCAATCCTATGTGCAAACCGTCGAGAATATG
ATTATGGCGTTAATTTCAATAAGTCTACCGGACTATAATACGCCCATGTTCGGCGACTCTTGGATCACTGATAAG
AATTTCAGAATGGCTCAATTTGCCTCATGGGCGCGTGTCTTCCCCGCTAACCAGGCAATTAAGTACTTTGCAACC
GACGGAAAACAGGGTAAAGCTCCCAACTTCTTGAGTAAGGCATTGTCCAATGCAGGATTCTACACGTTTAGAAGT
GGATGGGATAAGAACGCGACAGTGATGGTACTGAAGGCAAGTCCGCCTGGTGAGTTTCACGCTCAGCCTGATAAC
GGAACTTTTGAACTGTTCATCAAGGGTAGGAACTTCACACCCGACGCCGGTGTATTCGTCTATTCCGGTGACGAG
GCGATTATGAAATTGAGGAACTGGTACAGGCAGACAAGGATTCACTCTACTCTAACTCTTGATAATCAGAATATG
GTCATAACGAAAGCACGTCAGAATAAATGGGAGACAGGCAATAACTTGGATGTCTTAACTTACACTAACCCGTCA
TACCCCAACTTAGATCACCAACGTTCAGTTCTATTCATTAACAAGAAGTATTTCTTAGTCATAGATAGGGCTATC
GGCGAGGCCACGGGCAACCTTGGCGTGCACTGGCAGCTAAAGGAAGACAGTAACCCTGTCTTCGATAAAACCAAG
AATAGAGTCTATACTACCTATAGGGACGGGAACAATCTAATGATACAGAGTCTAAACGCAGACAGGACATCACTG
AACGAGGAAGAAGGGAAGGTATCTTATGTCTATAACAAGGAGCTGAAGCGTCCAGCTTTCGTATTTGAGAAGCCT
AAGAAGAACGCAGGGACCCAAAACTTTGTGTCAATTGTCTATCCATACGACGGTCAGAAGGCGCCGGAAATTTCT
ATTAGAGAAAATAAGGGAAATGACTTCGAAAAGGGAAAATTGAACCTAACACTGACTATAAACGGTAAGCAACAG
CTTGTGTTGGTTCCTGGATCCGCCATTTCTCAAATCACTGACGGTCAAATCCAAGCTACTACCACTGCTACCACC
GAAGCTACCACCACTGCTGCCCCATCTTCCACCGTTGAAACTGTTTCTCCATCCAGCACCGAAACTATCTCTCAA
CAAACTGAAAATGGTGCTGCTAAGGCCGCTGTCGGTATGGGTGCCGGTGCTCTAGCTGCTGCTGCTATGTTGTTA
TAA
SEQ ID NO: 12 is a reencoded nucleic acid sequence of glucuronosyl-
disulfoglucosamine glucuronidase originating from Pedobacter heparinus with a N-terminal
secretion sigal
ATGCAGTTTTCAACCGTGGCATCTGTGGCATTCGTGGCATTGGCAAATTTTGTCGCTGCTCAATCTTCCAGTATT
ACGCGTAAAGATTTTGACCACATAAATCTTGAGTATAGTGGCCTAGAGAAAGTCAATAAAGCTGTCGCCGCTGGT
AACTATGATGATGCAGCCAAGGCTTTGTTAGCCTATTATAGAGAGAAATCTAAGGCTCGTGAACCGGATTTCTCT
AACGCTGAAAAGCCAGCGGATATCAGACAACCAATCGATAAAGTCACCAGGGAAATGGCAGACAAGGCTTTGGTT
CACCAATTCCAGCCCCATAAGGGCTATGGTTACTTCGACTACGGTAAAGACATAAACTGGCAAATGTGGCCCGTA
AAGGATAATGAAGTAAGGTGGCAACTTCACAGAGTGAAGTGGTGGCAAGCTATGGCTCTGGTGTATCACGCAACC
GGTGATGAAAAGTATGCTAGAGAATGGGTATATCAGTATTCTGATTGGGCTAGGAAGAATCCACTAGGACTTTCT
CAGGATAACGACAAATTCGTTTGGCGTCCACTAGAGGTATCTGACAGGGTACAATCTTTACCCCCGACGTTCAGT
CTGTTTGTTAATTCTCCAGCATTTACCCCGGCTTTCTTGATGGAATTCTTGAATTCTTATCATCAGCAAGCGGAT
TATTTAAGCACCCATTATGCAGAACAAGGAAATCACAGATTATTTGAGGCCCAACGTAATTTATTTGCGGGTGTT
AGTTTTCCAGAGTTTAAAGATTCGCCAAGATGGAGACAAACCGGTATCAGCGTGTTAAACACTGAAATCAAGAAA
CAAGTATATGCTGACGGTATGCAGTTTGAACTATCTCCAATTTATCATGTCGCAGCAATTGACATATTCTTGAAG
GCTTATGGCAGTGCTAAACGTGTCAACTTAGAAAAGGAATTTCCACAGTCATACGTCCAGACTGTAGAAAACATG
ATCATGGCCCTTATCAGCATCTCATTGCCTGATTACAACACTCCAATGTTTGGTGATAGTTGGATAACGGACAAG
AACTTTAGGATGGCACAGTTCGCAAGCTGGGCTAGAGTATTTCCAGCAAATCAAGCTATAAAATATTTCGCGACT
GATGGTAAGCAAGGGAAGGCCCCTAATTTTCTATCTAAAGCTTTAAGCAACGCCGGCTTTTATACTTTCAGGTCC
GGTTGGGACAAGAATGCAACTGTCATGGTGTTAAAAGCGTCTCCACCCGGAGAATTCCATGCGCAACCAGACAAT
GGGACATTCGAGTTGTTTATTAAAGGGAGAAATTTTACCCCAGATGCTGGAGTGTTTGTGTACTCAGGGGATGAA
GCGATAATGAAGTTAAGAAATTGGTATCGTCAAACCAGAATACATTCCACACTTACACTGGACAACCAAAACATG
GTGATTACTAAGGCTAGACAAAACAAGTGGGAAACTGGTAACAATCTGGACGTTTTGACATATACGAACCCTTCC
TATCCAAATCTTGACCATCAGAGGTCGGTCTTATTTATAAATAAGAAATACTTCTTGGTTATCGACAGAGCCATT
GGTGAAGCAACAGGTAATCTGGGTGTTCATTGGCAATTGAAAGAGGATTCTAATCCAGTGTTTGACAAGACGAAG
AACAGGGTATACACAACATACCGTGATGGTAATAACTTAATGATCCAGTCCTTAAATGCTGATCGTACTTCTCTT
AATGAAGAGGAAGGTAAGGTTTCCTACGTGTACAATAAAGAACTAAAGAGACCCGCCTTTGTCTTCGAAAAGCCC
AAGAAGAATGCCGGAACGCAGAATTTCGTATCTATCGTTTACCCTTATGATGGCCAGAAAGCTCCAGAGATAAGC
ATCCGTGAGAACAAAGGTAACGATTTTGAGAAAGGTAAGCTTAATTTGACGCTAACGATTAATGGGAAACAGCAA
TTAGTCTTAGTACCATAA
SEQ ID NO: 13 is a reencoded nucleic acid sequence of glucuronosyl-
disulfoglucosamine glucuronidase originating from Pedobacter heparinus with a N-terminal
secretion signal and a C-terminal anchoring signal
ATGCAGTTTTCAACCGTGGCATCTGTGGCATTCGTGGCATTGGCAAATTTTGTCGCTGCTCAATCTTCCAGTATT
ACGCGTAAAGATTTTGACCACATAAATCTTGAGTATAGTGGCCTAGAGAAAGTCAATAAAGCTGTCGCCGCTGGT
AACTATGATGATGCAGCCAAGGCTTTGTTAGCCTATTATAGAGAGAAATCTAAGGCTCGTGAACCGGATTTCTCT
AACGCTGAAAAGCCAGCGGATATCAGACAACCAATCGATAAAGTCACCAGGGAAATGGCAGACAAGGCTTTGGTT
CACCAATTCCAGCCCCATAAGGGCTATGGTTACTTCGACTACGGTAAAGACATAAACTGGCAAATGTGGCCCGTA
AAGGATAATGAAGTAAGGTGGCAACTTCACAGAGTGAAGTGGTGGCAAGCTATGGCTCTGGTGTATCACGCAACC
GGTGATGAAAAGTATGCTAGAGAATGGGTATATCAGTATTCTGATTGGGCTAGGAAGAATCCACTAGGACTTTCT
CAGGATAACGACAAATTCGTTTGGCGTCCACTAGAGGTATCTGACAGGGTACAATCTTTACCCCCGACGTTCAGT
CTGTTTGTTAATTCTCCAGCATTTACCCCGGCTTTCTTGATGGAATTCTTGAATTCTTATCATCAGCAAGCGGAT
TATTTAAGCACCCATTATGCAGAACAAGGAAATCACAGATTATTTGAGGCCCAACGTAATTTATTTGCGGGTGTT
AGTTTTCCAGAGTTTAAAGATTCGCCAAGATGGAGACAAACCGGTATCAGCGTGTTAAACACTGAAATCAAGAAA
CAAGTATATGCTGACGGTATGCAGTTTGAACTATCTCCAATTTATCATGTCGCAGCAATTGACATATTCTTGAAG
GCTTATGGCAGTGCTAAACGTGTCAACTTAGAAAAGGAATTTCCACAGTCATACGTCCAGACTGTAGAAAACATG
ATCATGGCCCTTATCAGCATCTCATTGCCTGATTACAACACTCCAATGTTTGGTGATAGTTGGATAACGGACAAG
AACTTTAGGATGGCACAGTTCGCAAGCTGGGCTAGAGTATTTCCAGCAAATCAAGCTATAAAATATTTCGCGACT
GATGGTAAGCAAGGGAAGGCCCCTAATTTTCTATCTAAAGCTTTAAGCAACGCCGGCTTTTATACTTTCAGGTCC
GGTTGGGACAAGAATGCAACTGTCATGGTGTTAAAAGCGTCTCCACCCGGAGAATTCCATGCGCAACCAGACAAT
GGGACATTCGAGTTGTTTATTAAAGGGAGAAATTTTACCCCAGATGCTGGAGTGTTTGTGTACTCAGGGGATGAA
GCGATAATGAAGTTAAGAAATTGGTATCGTCAAACCAGAATACATTCCACACTTACACTGGACAACCAAAACATG
GTGATTACTAAGGCTAGACAAAACAAGTGGGAAACTGGTAACAATCTGGACGTTTTGACATATACGAACCCTTCC
TATCCAAATCTTGACCATCAGAGGTCGGTCTTATTTATAAATAAGAAATACTTCTTGGTTATCGACAGAGCCATT
GGTGAAGCAACAGGTAATCTGGGTGTTCATTGGCAATTGAAAGAGGATTCTAATCCAGTGTTTGACAAGACGAAG
AACAGGGTATACACAACATACCGTGATGGTAATAACTTAATGATCCAGTCCTTAAATGCTGATCGTACTTCTCTT
AATGAAGAGGAAGGTAAGGTTTCCTACGTGTACAATAAAGAACTAAAGAGACCCGCCTTTGTCTTCGAAAAGCCC
AAGAAGAATGCCGGAACGCAGAATTTCGTATCTATCGTTTACCCTTATGATGGCCAGAAAGCTCCAGAGATAAGC
ATCCGTGAGAACAAAGGTAACGATTTTGAGAAAGGTAAGCTTAATTTGACGCTAACGATTAATGGGAAACAGCAA
TTAGTCTTAGTACCATAA
SEQ ID NO: 14 is an amino acid sequence of glucuronosyl-disulfoglucosamine
glucuronidase originating from Pedobacter heparinus with a N-terminal secretion signal
MQFSTVASVAFVALANFVAAQSSSITRKDFDHINLEYSGLEKVNKAVAAGNYDDAAKALLAYYREKSKAREPDFS
NAEKPADIRQPIDKVTREMADKALVHQFQPHKGYGYFDYGKDINWQMWPVKDNEVRWQLHRVKWWQAMALVYHAT
GDEKYAREWVYQYSDWARKNPLGLSQDNDKFVWRPLEVSDRVQSLPPTFSLFVNSPAFTPAFLMEFLNSYHQQAD
YLSTHYAEQGNHRLFEAQRNLFAGVSFPEFKDSPRWRQTGISVLNTEIKKQVYADGMQFELSPIYHVAAIDIFLK
AYGSAKRVNLEKEFPQSYVQTVENMIMALISISLPDYNTPMFGDSWITDKNFRMAQFASWARVFPANQAIKYFAT
DGKQGKAPNFLSKALSNAGFYTFRSGWDKNATVMVLKASPPGEFHAQPDNGTFELFIKGRNFTPDAGVFVYSGDE
AIMKLRNWYRQTRIHSTLILDNQNMVITKARQNKWETGNNLDVLTYTNPSYPNLDHQRSVLFINKKYFLVIDRAI
GEATGNLGVHWQLKEDSNPVFDKTKNRVYTTYRDGNNLMIQSLNADRTSLNEEEGKVSYVYNKELKRPAFVFEKP
KKNAGTQNFVSIVYPYDGQKAPEISIRENKGNDFEKGKLNLTLTINGKQQLVLVP
SEQ ID NO: 15 is an amino acid sequence of glucuronosyl-disulfoglucosamine
glucuronidase originating from Pedobacter heparinus with a N-terminal secretion signal and
a C-terminal anchoring signal
MQFSTVASVAFVALANFVAAQSSSITRKDFDHINLEYSGLEKVNKAVAAGNYDDAAKALLAYYREKSKAREPDFS
NAEKPADIRQPIDKVTREMADKALVHQFQPHKGYGYFDYGKDINWQMWPVKDNEVRWQLHRVKWWQAMALVYHAT
GDEKYAREWVYQYSDWARKNPLGLSQDNDKFVWRPLEVSDRVQSLPPTFSLFVNSPAFTPAFLMEFLNSYHQQAD
YLSTHYAEQGNHRLFEAQRNLFAGVSFPEFKDSPRWRQTGISVLNTEIKKQVYADGMQFELSPIYHVAAIDIFLK
AYGSAKRVNLEKEFPQSYVQTVENMIMALISISLPDYNTPMFGDSWITDKNFRMAQFASWARVFPANQAIKYFAT
DGKQGKAPNFLSKALSNAGFYTFRSGWDKNATVMVLKASPPGEFHAQPDNGTFELFIKGRNFTPDAGVFVYSGDE
AIMKLRNWYRQTRIHSTLILDNQNMVITKARQNKWETGNNLDVLTYTNPSYPNLDHQRSVLFINKKYFLVIDRAI
GEATGNLGVHWQLKEDSNPVFDKTKNRVYTTYRDGNNLMIQSLNADRTSLNEEEGKVSYVYNKELKRPAFVFEKP
KKNAGTQNFVSIVYPYDGQKAPEISIRENKGNDFEKGKLNLTLTINGKQQLVLVPGSAISQITDGQIQATTTATT
EATTTAAPSSTVETVSPSSTETISQQTENGAAKAAVGMGAGALAAAAMLL
SEQ ID NO: 16 is a nucleic acid sequence of glutamine-fructose-6-phosphate
amidotransferase (GFA1) originating from Saccharomyces cerevisiae
ATGTGTGGTATCTTTGGTTACTGCAATTATCTAGTGGAAAGATCCAGAGGAGAAATTATCGACACCTTAGTGGAT
GGTTTACAAAGATTAGAATATAGAGGCTATGATTCCACCGGTATTGCTATCGATGGTGACGAAGCTGATTCTACT
TTCATCTATAAGCAAATCGGTAAAGTGAGTGCTTTGAAAGAGGAGATTACTAAGCAAAATCCGAACAGAGACGTT
ACTTTTGTCTCTCATTGTGGTATTGCGCATACTAGATGGGCTACTCACGGTCGACCAGAACAAGTTAACTGTCAC
CCTCAAAGATCTGACCCAGAAGACCAATTTGTGGTCGTTCATAATGGTATCATCACAAATTTTAGAGAACTGAAG
ACTCTTTTAATTAACAAAGGTTATAAATTCGAAAGTGATACCGATACCGAGTGTATTGCTAAACTATATTTGCAT
TTATACAATACAAATTTACAAAATGGGCATGACTTAGATTTCCACGAATTAACCAAGCTAGTTCTTTTAGAACTA
GAAGGTTCATACGGGTTATTATGTAAATCTTGTCACTATCCTAATGAGGTTATCGCCACTAGAAAAGGGTCCCCT
TTACTGATTGGTGTCAAATCTGAAAAAAAACTAAAAGTCGACTTCGTGGATGTGGAATTTCCCGAAGAAAACGCT
GGTCAACCGGAAATTCCATTGAAATCTAACAACAAATCATTTGGCTTGGGCCCAAAGAAAGCTCGTGAATTTGAA
GCTGGTTCCCAAAATGCCAATTTACTACCAATTGCCGCCAATGAATTTAACTTGAGACATTCTCAATCCAGGGCT
TTCCTATCAGAAGATGGATCTCCAACACCGGTGGAATTTTTTGTTTCTTCGGATGCGGCATCTGTTGTTAAACAT
ACCAAGAAGGTGCTATTTTTAGAAGATGACGATTTGGCTCATATTTACGATGGTGAGTTACATATTCATAGATCT
AGAAGAGAAGTAGGCGCATCAATGACAAGGTCCATTCAAACTTTAGAGATGGAGTTAGCTCAGATCATGAAGGGC
CCTTACGACCATTTTATGCAAAAGGAAATCTATGAGCAACCAGAATCTACTTTCAATACTATGAGAGGTAGAATC
GACTATGAAAATAATAAAGTGATATTGGGTGGTTTAAAGGCATGGTTACCAGTTGTCAGAAGAGCACGGAGACTG
ATCATGATCGCATGCGGTACTTCTTATCATTCATGTTTGGCTACTCGTGCTATCTTCGAAGAATTATCAGATATC
CCAGTTAGTGTGGAATTAGCGTCTGACTTTCTGGACAGAAAATGCCCTGTCTTCAGAGACGATGTATGCGTGTTT
GTTTCACAAAGTGGTGAAACTGCGGATACCATGCTGGCTCTAAATTATTGTTTAGAAAGAGGAGCCTTAACTGTC
GGAATTGTTAACAGTGTTGGTTCTTCTATCTCTCGTGTCACCCACTGTGGTGTTCATATTAACGCTGGTCCTGAA
ATTGGTGTTGCCTCTACAAAAGCTTATACTTCCCAGTATATTGCCTTAGTGATGTTTGCTCTATCGCTGTCAGAT
GACCGTGTATCGAAAATAGACAGAAGAATTGAAATCATTCAAGGCTTGAAGTTAATCCCGGGCCAAATTAAGCAG
GTATTAAAGCTGGAACCAAGAATAAAAAAGCTCTGTGCGACTGAATTAAAGGATCAAAAATCTCTATTGTTATTG
GGTAGAGGTTACCAATTTGCTGCTGCTCTGGAAGGTGCTTTGAAGATCAAAGAAATTTCTTATATGCATTCTGAA
GGTGTTTTGGCAGGTGAGTTGAAGCACGGTGTCTTGGCCTTGGTGGACGAAAACTTGCCAATCATTGCTTTTGGT
ACCAGAGACTCTCTATTCCCTAAAGTAGTTTCCTCTATTGAGCAAGTTACTGTAAGAAAGGGCCATCCAATTATT
ATTTGTAACGAAAATGATGAAGTGTGGGCGCAAAAATCTAAATCAATCGACCTGCAAACCTTAGAAGTTCCACAA
ACTGTTGATTGTTTACAAGGTCTAATTAATATTATTCCATTACAACTAATGTCATATTGGTTGGCTGTTAATAAA
GGGATTGATGTTGATTTTCCAAGAAACTTGGCTAAATCTGTTACCGTCGAATAA
SEQ ID NO: 17 is a reencoded nucleic acid sequence of glutamine-fructose-6-
phosphate amidotransferase (GFA1) originating from Chlorella virus 1 (PBCV-1)
ATGTGCGGGATCTTCGGTGCTGTGTCGAACAATAATAGCATAGAAGTTTCCATCAAGGGTATACAGAAGCTAGAG
TACCGCGGGTACGATTCGTGTGGAATAGCCTATACAGACGGAGGCGCCATTGAACGGATCAGGTCAATAGATGGG
ATCGACGACTTAAGGAAGAAAACAATAACAGAGTCTTCCCCGGTAGCTATCGCTCATTCAAGGTGGAGTACCACT
GGGATTCCAAGTGTTGTGAACGCCCACCCGCACATCTCTCGGGGCACGTCTGGATGCGAGTCGCGCATTGCAGTC
GTACACAATGGCATTATTGAAAATTACCAGCAGATCCGCAAGTACTTGATAAATCTTGGGTATACTTTCGATTCA
CAGACCGACACGGAAGTCATTGCTCATCTAATAGACTCGCAGTATAACGGGAACATCCTGCACACCGTCCAAATG
GCAGTTAAGCACCTCAAAGGAAGCTACGCCATAGCAGTCATGTGCCATAAGGAGTCCGGGAAGATCGTGGTGGCA
AAACAGAAGAGTCCGTTGGTATTGGGTATAGGAAGCGACGGAGCTTATTATATCGCATCGGACGTATTGGCTCTT
CCCACTAACAAAGTGGTCTACATAAGTGACGGATTCTCTGCTGAGTTAAGTCCTGGATCGATGACTATCTATGAT
CTGGATGGCAATAAGGTAGAATACGAGGTGGAAGACGTTGAAATGGAGCAGACGTCGATGTCTTTAGATAACTTT
GATCACTACATGATCAAAGAGATCAATGAACAGCCGATATCAATTCTCAATACTATTAAGAACAAAGGATTCTAT
GCAGAGATATTTGGTGATCTAGCACATGAAATATTTCAGAAGATCGACAATATATTGATTCTAGCCTGCGGAACT
TCGTATCATGCAGGTCTCGTCGGGAAGCAATGGATAGAGACCATCTCACGCATCCCGGTGGATGTTCATATTGCA
AGCGAGTATGAGCCGACAATTCCACGAGCCAATACACTTGTGATAACGATTAGTCAAAGCGGCGAGACTGCGGAT
ACAATCGCAGCCCTTCAACGAGCGCAAAACGCAGGAATGATATACACTTTGTGTATCTGCAACAGCCCCAAGTCA
ACTCTAGTGCGCGAGTCGATAATGAAATACATAACCAAATGCGGTTCAGAAGTTTCTGTTGCCAGTACTAAAGCA
TTTACGTCGCAACTTGTGGTGTTGTATATGCTTGCGAACGTTCTTGCGAACAAAACTGATGATCTGCTAGGGGAT
CTTCCTCAAGCTATCGAGAGAGTAATCTGTCTTACTAATGACGAAATGAAGCGTTGGGCGGATGAAATTTGTACT
GCCAAAAGTGCCATCTTCTTAGGGAGAGGACTGAACGCACCTGTAGCATTTGAGGGCGCGCTAAAGTTGAAGGAA
ATCTCATATATCCATGCCGAGGGTTTCCTCGGTGGAGAGTTGAAGCATGGGCCGCTGGCTTTGTTGGACGATAAG
ATCCCTGTAATCGTGACTGTTGCTGACCACGCTTATTTGGATCACATTAAGGCTAACATCGACGAGGTATTGGCA
CGAAACGTTACCGTATACGCGATCGTCGATCAGTATGTTAATATTGAACCACAGGAGCGACTGCATGTAGTAAAA
GTGCCGTTTGTGAGTAAAGAGTTTTCTCCCATCATCCACACAATTCCGATGCAATTGTTATCGTATTATGTCGCG
ATCAAGCTGGGCAAGAACGTTGACAAGCCACGTAACCTGGCGAAAAGTGTTACAACATTCtaa
SEQ ID NO: 18 is a reencoded nucleic acid sequence of glutamine-fructose-6-
phosphate amidotransferase (GFA1) originating from Chlorella virus 1 (PBCV-1)
ATGTGTGGCATCTTTGGAGCAGTGTCAAACAACAACTCTATCGAGGTGTCAATCAAGGGTATTCAGAAGCTAGAA
TATCGTGGGTATGATTCGTGCGGTATTGCGTATACAGATGGGGGTGCGATTGAGCGTATACGTTCTATTGACGGC
ATTGACGATCTGCGTAAGAAAACAATCACAGAATCATCACCAGTGGCCATTGCTCACTCGCGGTGGAGCACCACT
GGAATTCCATCAGTGGTGAACGCACATCCTCATATTTCTCGCGGAACCAGTGGGTGTGAGTCTCGTATCGCGGTA
GTCCACAACGGTATCATTGAAAACTATCAGCAGATCCGAAAATATCTCATCAATCTTGGTTATACGTTTGATAGT
CAAACGGACACAGAGGTCATTGCGCATTTGATTGATTCTCAGTACAATGGGAATATCTTGCACACCGTCCAAATG
GCTGTCAAGCACCTGAAGGGCTCTTATGCCATTGCAGTTATGTGTCATAAAGAGTCTGGTAAAATAGTCGTGGCG
AAACAGAAGTCACCCCTCGTACTTGGAATCGGCTCAGATGGTGCTTACTACATCGCTTCGGACGTGCTGGCGCTG
CCGACAAATAAAGTTGTTTATATTTCAGACGGTTTCTCCGCAGAACTATCTCCAGGGAGTATGACCATTTACGAT
CTTGATGGAAATAAAGTAGAATATGAAGTAGAGGACGTTGAAATGGAACAAACTAGTATGTCTCTCGATAACTTT
GATCATTACATGATTAAGGAAATTAATGAGCAACCAATCAGTATTCTAAACACTATAAAAAATAAAGGGTTCTAT
GCAGAAATATTCGGTGATTTGGCTCATGAAATCTTCCAAAAAATAGACAACATCCTGATACTGGCTTGTGGTACA
AGTTATCACGCCGGTCTTGTAGGAAAACAGTGGATAGAGACCATCTCLAGAATCCCCGTGGATGTTCACATCGCG
AGTGAATACGAACCTACTATTCCGAGAGCGAACACATTGGTAATCACTATTTCACAGTCGGGTGAAACTGCGGAC
ACGATAGCGGCTTTGCAACGGGCCCAAAACGCCGGGATGATTTATACATTGTGTATTTGCAATTCACCAAAGAGC
ACTCTTGTTCGTGAGAGCATTATGAAGTACATCACGAAATGTGGTTCTGAGGTGTCAGTGGCATCAACGAAGGCG
TTTACTTCTCAGCTCGTAGTACTGTACATGCTGGCAAACGTATTGGCAAATAAAACCGATGATTTGCTGGGAGAC
CTCCCACAGGCAATAGAACGGGTAATTTGTTTGACAAATGACGAAATGAAACGATGGGCCGACGAAATTTGCACT
GCGAAATCCGCGATCTTCCTGGGAAGAGGACTAAACGCACCAGTTGCCTTTGAGGGAGCGTTGAAGCTCAAAGAA
ATCTCTTACATTCATGCAGAGGGCTTCCTGGGAGGTGAGTTGAAACACGGCCCCCTCGCACTCCTTGATGACAAG
ATTCCTGTTATCGTAACCGTAGCAGATCATGCTTATTTGGACCATATCAAAGCAAATATCGACGAAGTGCTTGCG
AGGAACGTTACGGTATACGCCATAGTAGACCAGTATGTGAACATCGAGCCCCAGGAACGCCTTCACGTCGTCAAG
GTTCCGTTTGTATCCAAAGAATTTTCTCCGATAATTCACACTATCCCGATGCAACTGCTTTCGTATTACGTGGCA
ATTAAGCTTGGAAAGAACGTTGACAAACCAAGGAATCTTGCAAAATCCGTGACTACCTTTTAA
SEQ ID NO: 19 is an amino acid sequence of glutamine-fructose-6-phosphate
amidotransferase (GFA1) originating from Saccharomyces cerevisiae
MCGIFGYCNYLVERSRGEIIDTLVDGLQRLEYRGYDSTGIAIDGDEADSTFIYKQIGKVSALKEEITKQNPNRDV
TFVSHCGIAHTRWATHGRPEQVNCHPQRSDPEDQFVVVHNGIITNFRELKILLINKGYKFESDTDTECIAKLYLH
LYNTNLONGHDLDFHELTKLVLLELEGSYGLLCKSCHYPNEVIATRKGSPLLIGVKSEKKLKVDFVDVEFPEENA
GQPEIPLKSNNKSFGLGPKKAREFEAGSQNANLLPIAANEFNLRHSQSRAFLSEDGSPTPVEFFVSSDAASVVKH
TKKVLFLEDDDLAHIYDGELHIHRSRREVGASMTRSIQTLEMELAQIMKGPYDHFMQKEIYEQPESTENTMRGRI
DYENNKVILGGLKAWLPVVRRARRLIMIACGTSYHSCLATRAIFEELSDIPVSVELASDFLDRKCPVFRDDVCVF
VSQSGETADTMLALNYCLERGALTVGIVNSVGSSISRVTHCGVHINAGPEIGVASTKAYTSQYIALVMFALSLSD
DRVSKIDRRIEIIQGLKLIPGQIKQVLKLEPRIKKLCATELKDQKSLLLLGRGYQFAAALEGALKIKEISYMHSE
GVLAGELKHGVLALVDENLPIIAFGTRDSLFPKVVSSIEQVTARKGHPIIICNENDEVWAQKSKSIDLQTLEVPQ
TVDCLQGLINIIPLQLMSYWLAVNKGIDVDFPRNLAKSVTVE
SEQ ID NO: 20 is an amino acid sequence of glutamine-fructose-6-phosphate
amidotransferase (GFA1) originating from Chlorella virus 1 (PBCV-1)
MCGIFGAVSNNNSIEVSIKGIQKLEYRGYDSCGIAYTDGGAIERIRSIDGIDDLRKKTITESSPVAIAHSRWSTT
GIPSVVNAHPHISRGTSGCESRIAVVHNGIIENYQQIRKYLINLGYTFDSQTDTEVIAHLIDSQYNGNILHTVQM
AVKHLKGSYAIAVMCHKESGKIVVAKQKSPLVLGIGSDGAYYIASDVLALPTNKVVYISDGFSAELSPGSMTIYD
LDGNKVEYEVEDVEMEQTSMSLDNFDHYMIKEINEQPISILNTIKNKGFYAEIFGDLAHEIFQKIDNILILACGT
SYHAGLVGKOWIETISRIPVDVHIASEYEPTIPRANTLVITISQSGETADTIAALQRAQNAGMIYTLCICNSPKS
TLVRESIMKYITKCGSEVSVASTKAFTSQLVVLYMLANVLANKTDDLLGDLPQAIERVICLINDEMKRWADEICT
AKSAIFLGRGLNAPVAFEGALKLKEISYIHAEGFLGGELKHGPLALLDDKIPVIVTVADHAYLDHIKANIDEVLA
RNVTVYAIVDQYVNIEPQERLHVVKVPFVSKEFSPIIHTIPMQLLSYYVAIKLGKNVDKPRNLAKSVTTF
SEQ ID NO: 21 is a nucleic acid sequence of UDP-N-acetylglucosamine
pyrophosphorylase (QRI1) originating from Saccharomyces cerevisiae
ATGACTGACACAAAACAGCTATTCATTGAAGCCGGACAAAGTCAACTTTTCCACAATTGGGAAAGCTTGTCTCGC
AAAGACCAAGAAGAATTGCTTTCAAACCTGGAGCAAATATCTTCCAAGAGGTCCCCTGCAAAACTACTGGAAGAC
TGTCAAAATGCTATTAAATTCTCACTAGCTAACTCTTCTAAGGATACTGGCGTCGAAATTTCACCATTGCCCCCT
ACTTCGTACGAGTCGCTTATTGGCAACAGTAAGAAAGAAAATGAATACTGGCGTTTAGGCCTTGAAGCTATTGGC
AAGGGTGAAGTCGCAGTGATTTTAATGGCTGGCGGACAAGGTACGCGGTTAGGATCCTCTCAACCAAAGGGCTGT
TACGACATTGGATTGCCTTCTAAGAAATCTCTTTTTCAAATTCAAGCTGAAAAGTTGATCAGGTTGCAAGATATG
GTAAAGGACAAAAAGGTAGAAATTCCTTGGTATATTATGACATCAGGCCCCACTAGAGCGGCTACTGAGGCATAC
TTTCAAGAACACAATTATTTTGGCTTGAATAAAGAACAAATTACGTTCTTCAACCAGGGAACCCTGCCTGCCTTT
GATTTAACCGGGAAGCATTTCCTAATGAAAGACCCAGTAAACCTATCTCAATCACCAGATGGAAATGGTGGACTC
TACCGTGCCATCAAGGAAAACAAGTTGAACGAAGACTTTGATAGGAGAGGAATCAAGCATGTTTACATGTACTGT
GTCGATAATGTCCTATCTAAAATCGCAGACCCTGTATTTATTGGTTTTGCCATCAAGCATGGCTTCGAACTGGCC
ACCAAAGCCGTTAGAAAGAGAGATGCGCATGAATCAGTTGGGTTAATTGCTACTAAAAACGAGAAACCATGTGTC
ATAGAATATTCTGAAATTTCCAATGAATTGGCTGAAGCAAAGGATAAAGATGGCTTATTAAAACTACGCGCAGGC
AACATTGTAAATCATTATTACCTAGTGGATTTACTAAAACGTGATTTGGATCAGTGGTGTGAGAATATGCCATAT
CACATTGCGAAGAAGAAAATTCCAGCTTATGATAGTGTTACCGGCAAGTACACTAAGCCTACCGAACCAAACGGT
ATAAAATTAGAGCAATTCATATTTGATGTCTTTGACACTGTACCACTGAACAAGTTTGGGTGCTTAGAAGTAGAT
AGATGCAAAGAATTTTCACCTTTAAAAAACGGTCCTGGTTCTAAGAACGATAATCCTGAGACCAGCAGACTAGCA
TATTTGAAACTAGGAACCTCGTGGTTGGAAGATGCAGGCGCTATTGTAAAAGATGGGGTACTAGTCGAAGTTTCC
AGCAAATTGAGTTATGCAGGTGAAAATCTATCCCAGTTCAAAGGTAAAGTCTTTGACAGAAGTGGTATAGTATTA
GAAAAATAA
SEQ ID NO: 22 is an amino acid sequence of UDP-N-acetylglucosamine
pyrophosphorylase (QRI1) originating from Saccharomyces cerevisiae
MTDTKQLFIEAGQSQLFHNWESLSRKDQEELLSNLEQISSKRSPAKLLEDCQNAIKFSLANSSKDTGVEISPLPP
TSYESLIGNSKKENEYWRLGLEAIGKGEVAVILMAGGQGTRLGSSQPKGCYDIGLPSKKSLFQIQAEKLIRLQDM
VKDKKVEIPWYIMISGPTRAATEAYFQEHNYFGLNKEQITFFNQGTLPAFDLTGKHFLMKDPVNLSQSPDGNGGL
YRAIKENKLNEDFDRRGIKHVYMYCVDNVLSKIADPVFIGFAIKHGFELATKAVRKRDAHESVGLIATKNEKPCV
IEYSEISNELAEAKDKDGLLKLRAGNIVNHYYLVDLLKRDLDQWCENMPYHIAKKKIPAYDSVTGKYTKPTEPNG
IKLEQFIFDVFDTVPLNKFGCLEVDRCKEFSPLKNGPGSKNDNPETSRLAYLKLGTSWLEDAGAIVKDGVLVEVS
SKLSYAGENLSQFKGKVFDRSGIVLEK
SEQ ID NO: 23 is a nucleic acid sequence of Phosphoglucomutase-1 (PGM1)
originating from Saccharomyces cerevisiae
ATGTCACTTCTAATAGATTCTGTACCAACAGTTGCTTATAAGGACCAAAAACCGGGTACTTCAGGTTTACGTAAG
AAGACCAAGGTTTTCATGGATGAGCCTCATTATACTGAGAACTTCATTCAAGCAACAATGCAATCTATCCCTAAT
GGCTCAGAGGGAACCACTTTAGTTGTTGGAGGAGATGGTCGTTTCTACAACGATGTTATCATGAACAAGATTGCC
GCAGTAGGTGCTGCAAACGGTGTCAGAAAGTTAGTCATTGGTCAAGGCGGTTTACTTTCAACACCAGCTGCTTCT
CATATAATTAGAACATACGAGGAAAAGTGTACCGGTGGTGGTATCATATTAACTGCCTCACACAACCCAGGCGGT
CCAGAGAATGATTTAGGTATCAAGTATAATTTACCTAATGGTGGGCCAGCTCCAGAGAGTGTCACTAACGCTATC
TGGGAAGCGTCTAAAAAATTAACTCACTATAAAATTATAAAGAACTTCCCCAAGTTGAATTTGAACAAGCTTGGT
AAAAACCAAAAATATGGCCCATTGTTAGTGGACATAATTGATCCTGCCAAAGCATACGTTCAATTTCTGAAGGAA
ATTTTTGATTTTGACTTAATTAAAAGCTTCTTAGCGAAACAGCGCAAAGACAAAGGGTGGAAGTTGTTGTTTGAC
TCCTTAAATGGTATTACAGGACCATATGGTAAGGCTATATTTGTTGATGAATTTGGTTTACCGGCAGAGGAAGTT
CTTCAAAATTGGCACCCTTTACCTGATTTCGGCGGTTTACATCCCGATCCGAATCTAACCTATGCACGAACTCTT
GTTGACAGGGTTGACCGCGAAAAAATTGCCTTTGGAGCAGCCTCCGATGGTGATGGTGATAGGAATATGATTTAC
GGTTATGGCCCTGCTTTCGTTTCGCCAGGTGATTCTGTTGCCATTATTGCCGAATATGCACCCGAAATTCCATAC
TTCGCCAAACAAGGTATTTATGGCTTGGCACGTTCATTTCCTACATCCTCAGCCATTGATCGTGTTGCAGCAAAA
AAGGGATTAAGATGTTACGAAGTTCCAACCGGCTGGAAATTCTTCTGTGCCTTATTTGATGCTAAAAAGCTATCA
ATCTGTGGTGAAGAATCCTTCGGTACAGGTTCCAATCATATCAGAGAAAAGGACGGTCTATGGGCCATTATTGCT
TGGTTAAATATCTTGGCTATCTACCATAGGCGTAACCCTGAAAAGGAAGCTTCGATCAAAACTATTCAGGACGAA
TTTTGGAACGAGTATGGCCGTACTTTCTTCACAAGATACGATTACGAACATATCGAATGCGAGCAGGCCGAAAAA
GTTGTAGCTCTTTTGAGTGAATTTGTATCAAGGCCAAACGTTTGTGGCTCCCACTTCCCAGCTGATGAGTCTTTA
ACCGTTATCGATTGTGGTGATTTTTCGTATAGAGATCTAGATGGCTCCATCTCTGAAAATCAAGGCCTTTTCGTA
AAGTTTTCGAATGGGACTAAATTTGTTTTGAGGTTATCCGGCACAGGCAGTTCTGGTGCAACAATAAGATTATAC
GTAGAAAAGTATACTGATAAAAAGGAGAACTATGGCCAAACAGCTGACGTCTTCTTGAAACCCGTCATCAACTCC
ATTGTAAAATTCTTAAGATTTAAAGAAATTTTAGGAACAGACGAACCAACAGTCCGCACATAG
SEQ ID NO: 24 is an amino acid sequence Phosphoglucomutase-1 (PGM1)
originating from Saccharomyces cerevisiae
MSLLIDSVPTVAYKDQKPGTSGLRKKTKVFMDEPHYTENFIQATMQSIPNGSEGTTLVVGGDGRFYNDVIMNKIA
AVGAANGVRKLVIGQGGLLSTPAASHIIRTYEEKCTGGGIILTASHNPGGPENDLGIKYNLPNGGPAPESVINAI
WEASKKLTHYKIIKNFPKLNLNKLGKNQKYGPLLVDIIDPAKAYVQFLKEIFDFDLIKSFLAKQRKDKGWKLLED
SLNGITGPYGKAIFVDEFGLPAEEVLONWHPLPDFGGLHPDPNLTYARTLVDRVDREKIAFGAASDGDGDRNMIY
GYGPAFVSPGDSVAIIAEYAPEIPYFAKQGIYGLARSFPTSSAIDRVAAKKGLRCYEVPTGWKFFCALFDAKKLS
ICGEESFGTGSNHIREKDGLWAIIAWLNILAIYHRRNPEKEASIKTIQDEFWNEYGRIFFTRYDYEHIECEQAEK
VVALLSEFVSRPNVCGSHFPADESLIVIDCGDFSYRDLDGSISENQGLFVKFSNGTKFVLRLSGTGSSGATIRLY
VEKYTDKKENYGQTADVFLKPVINSIVKFLRFKEILGTDEPTVRT
SEQ ID NO: 25 is a nucleic acid sequence of UTP-glucose-1-phosphate
uridylyltransferase (UGP1) originating from Saccharomyces cerevisiae
ATGTCCACTAAGAAGCACACCAAAACACATTCCACTTATGCATTCGAGAGCAACACAAACAGCGTTGCTGCCTCA
CAAATGAGAAACGCCTTAAACAAGTTGGCGGACTCTAGTAAACTTGACGATGCTGCTCGCGCTAAGTTTGAGAAC
GAACTGGATTCGTTTTTCACGCTTTTCAGGAGATATTTGGTAGAGAAGTCTTCTAGAACCACCTTGGAATGGGAC
AAGATCAAGTCTCCCAACCCGGATGAAGTGGTTAAGTATGAAATTATTTCTCAGCAGCCCGAGAATGTCTCAAAC
CTTTCCAAATTGGCTGTTTTGAAGTTGAACGGTGGGCTGGGTACCTCCATGGGCTGCGTTGGCCCTAAATCTGTT
ATTGAAGTGAGAGAGGGAAACACCTTTTTGGATTTGTCTGTTCGTCAAATTGAATACTTGAACAGACAGTACGAT
AGCGACGTGCCATTGTTATTGATGAATTCTTTCAACACTGACAAGGATACGGAACACTTGATTAAGAAGTATTCC
GCTAACAGAATCAGAATCAGATCTTTCAATCAATCCAGGTTCCCAAGAGTCTACAAGGATTCTTTATTGCCTGTC
CCCACCGAATACGATTCTCCACTGGATGCTTGGTATCCACCAGGTCACGGTGATTTGTTTGAATCTTTACACGTA
TCTGGTGAACTGGATGCCTTAATTGCCCAAGGAAGAGAAATATTATTTGTTTCTAACGGTGACAACTTGGGTGCT
ACCGTCGACTTAAAAATTTTAAACCACATGATCGAGACTGGTGCCGAATATATAATGGAATTGACTGATAAGACC
AGAGCCGATGTTAAAGGTGGTACTTTGATTTCTTACGATGGTCAAGTCCGTTTATTGGAAGTCGCCCAAGTTCCA
AAAGAACACATTGACGAATTCAAAAATATCAGAAAGTTTACCAACTTCAACACGAATAACTTATGGATCAATCTG
AAAGCAGTAAAGAGGTTGATCGAATCGAGCAATTTGGAGATGGAAATCATTCCAAACCAAAAAACTATAACAAGA
GACGGTCATGAAATTAATGTCTTACAATTAGAAACCGCTTGTGGTGCTGCTATCAGGCATTTTGATGGTGCTCAC
GGTGTTGTCGTTCCAAGATCAAGATTCTTGCCTGTCAAGACCTGTTCCGATTTGTTGCTGGTTAAATCAGATCTA
TTCCGTCTGGAACACGGTTCTTTGAAGTTAGACCCATCCCGTTTTGGTCCAAACCCATTAATCAAGTTGGGCTCG
CATTTCAAAAAGGTTTCTGGTTTTAACGCAAGAATCCCTCACATCCCAAAAATCGTCGAGCTAGATCATTTGACC
ATCACTGGTAACGTCTTTTTAGGTAAAGATGTCACTTTGAGGGGTACTGTCATCATCGTTTGCTCCGACGGTCAT
AAAATCGATATTCCAAACGGCTCCATATTGGAAAATGTTGTCGTTACTGGTAATTTGCAAATCTTGGAACATTGA
SEQ ID NO: 26 is an amino acid sequence of UTP-- lucose-1-phosphate
uridylyltransferase (UGP1) originating from Saccharomyces cerevisiae
MSTKKHTKTHSTYAFESNTNSVAASQMRNALNKLADSSKLDDAARAKFENELDSFFTLFRRYLVEKSSRTTLEWD
KIKSPNPDEVVKYEIISQQPENVSNLSKLAVLKLNGGLGTSMGCVGPKSVIEVREGNTFLDLSVRQIEYLNRQYD
SDVPLLLMNSFNTDKDTEHLIKKYSANRIRIRSFNQSRFPRVYKDSLLPVPTEYDSPLDAWYPPGHGDLFESLHV
SGELDALIAQGREILFVSNGDNLGATVDLKILNHMIETGAEYIMELTDKTRADVKGGTLISYDGQVRLLEVAQVP
KEHIDEFKNIRKFTNFNINNLWINLKAVKRLIESSNLEMEIIPNQKTITRDGHEINVLQLETACGAAIRHFDGAH
GVVVPRSRFLPVKTCSDLLLVKSDLFRLEHGSLKLDPSRFGPNPLIKLGSHFKKVSGFNARIPHIPKIVELDHLT
ITGNVFLGKDVTLRGTVIIVCSDGHKIDIPNGSILENVVVTGNLQILEH
SEQ ID NO: 27 is a nucleic acid sequence of Glucosamine 6-phosphate N-
acetyltransferase (GNA1) originating from Saccharomyces cerevisiae
ATGAGCTTACCCGATGGATTTTATATAAGGCGAATGGAAGAGGGGGATTTGGAACAGGTCACTGAGACGCTAAAG
GTTTTGACCACCGTGGGCACTATTACCCCCGAATCCTTCAGCAAACTCATAAAATACTGGAATGAAGCCACAGTA
TGGAATGATAACGAAGATAAAAAAATAATGCAATATAACCCCATGGTGATTGTGGACAAGCGCACCGAGACGGTT
GCCGCTACGGGGAATATCATCATCGAAAGAAAGATCATTCATGAACTGGGGCTATGTGGCCACATCGAGGACATT
GCAGTAAACTCCAAGTATCAGGGCCAAGGTTTGGGCAAGCTCTTGATTGATCAATTGGTAACTATCGGCTTTGAC
TACGGTTGTTATAAGATTATTTTAGATTGCGATGAGAAAAATGTCAAATTCTATGAAAAATGTGGGTTTAGCAAC
GCAGGCGTGGAAATGCAAATTAGAAAATAG
SEQ ID NO: 28 is an amino acid sequence of Glucosamine 6-phosphate N-
acetyltransferase (GNA1) originating from Saccharomyces cerevisiae
MSLPDGFYIRRMEEGDLEQVTETLKVLTTVGTITPESFSKLIKYWNEATVWNDNEDKKIMQYNPMVIVDKRTETV
AATGNIIIERKIIHELGLCGHIEDIAVNSKYQGQGLGKLLIDQLVTIGFDYGCYKIILDCDEKNVKFYEKCGFSN
AGVEMQIRK
SEQ ID NO: 29 is a nucleic acid sequence of phosphoacetylglucosamine
mutase (PCM1) originating from Saccharomyces cerevisiae
ATGAAGGTTGATTACGAGCAATTGTGCAAACTCTACGATGACACGTGCCGCACAAAGAATGTGCAGTTCAGTTAC
GGTACGGCCGGATTCAGAACGCTGGCCAAGAATTTGGATACGGTGATGTTCAGTACTGGTATACTGGCGGTTCTC
AGGTCGCTGAAGCTTCAGGGTCAGTATGTGGGGGTGATGATCACGGCGTCGCACAACCCATACCAGGACAACGGG
GTCAAGATCGTGGAACCAGACGGATCGATGCTTTTGGCCACATGGGAGCCATATGCCATGCAGTTGGCCAATGCG
GCCTCTTTTGCCACTAATTTTGAAGAATTTCGTGTTGAGTTGGCCAAGCTGATTGAACACGAAAAGATTGATTTG
AATACAACCGTCGTGCCTCACATCGTGGTTGGGAGAGACTCTAGGGAAAGTAGTCCATACTTGCTGCGCTGCTTG
ACTTCCTCCATGGCCAGCGTCTTCCACGCGCAAGTTTTGGACCTAGGCTGTGTCACTACGCCTCAATTGCATTAC
ATTACTGATTTGTCCAACAGGCGGAAACTGGAAGGAGACACAGCGCCAGTTGCCACAGAACAGGACTACTATTCG
TTCTTTATAGGAGCCTTCAACGAGCTCTTCGCCACGTATCAGCTGGAGAAGAGGCTGTCTGTCCCAAAATTGTTC
ATAGACACAGCCAATGGTATCGGTGGTCCACAGTTGAAAAAACTACTGGCCTCCGAAGATTGGGACGTGCCAGCG
GAGCAAGTTGAGGTAATCAACGACAGGTCCGATGTTCCAGAACTGTTGAATTTTGAATGCGGTGCGGATTATGTG
AAGACTAACCAGAGATTACCCAAGGGTCTTTCTCCATCCTCGTTTGATTCGCTATATTGCTCCTTTGATGGTGAC
GCAGACAGGGTTGTGTTCTACTATGTCGACTCAGGATCAAAATTTCATTTGTTGGATGGTGACAAAATTTCCACT
TTGTTTGCAAAGTTCTTGTCTAAACAACTAGAATTGGCACACCTAGAACATTCTTTGAAGATTGGTGTTGTGCAA
ACTGCCTATGCAAACGGCAGTTCCACCGCTTACATAAAAAATACGTTGCACTGTCCCGTGTCTTGCACTAAGACA
GGTGTTAAACACTTGCATCATGAAGCTGCCACTCAGTACGATATTGGCATTTATTTCGAAGCAAATGGACATGGT
ACGATTATATTCAGCGAAAAATTTCATCGAACTATCAAATCTGAATTATCCAAGTCCAAGTTAAATGGTGATACG
TTAGCTTTGAGAACTTTGAAGTGTTTCTCTGAATTGATTAATCAGACCGTGGGAGATGCTATTTCAGACATGCTT
GCTGTCCTTGCTACTTTGGCGATTTTGAAAATGTCGCCAATGGATTGGGATGAAGAGTATACTGATTTGCCCAAC
AAGCTGGTTAAGTGCATCGTTCCTGATAGGTCAATTTTCCAAACCACGGACCAGGAAAGAAAATTGCTCAATCCA
GTGGGGTTGCAAGACAAGATAGATCTTGTGGTAGCCAAGTATCCCATGGGAAGAAGCTTTGTCAGAGCCAGTGGT
ACGGAGGATGCGGTGAGGGTTTATGCGGAATGTAAGGACTCCTCTAAGTTAGGTCAATTTTGTGACGAAGTGGTG
GAGCACGTTAAGGCATCTGCTTGA
SEQ ID NO: 30 is an amino acid sequence of phosphoacetylglucosamine
mutase (PCM1) originating from Saccharomyces cerevisiae
MKVDYEQLCKLYDDTCRTKNVQFSYGTAGFRTLAKNLDTVMESTGILAVLRSLKLQGQYVGVMITASHNPYQDNG
VKIVEPDGSMLLATWEPYAMQLANAASFATNFEEFRVELAKLIEHEKIDLNTTVVPHIVVGRDSRESSPYLLRCL
TSSMASVFHAQVLDLGCVTTPQLHYITDLSNRRKLEGDTAPVATEQDYYSFFIGAFNELFATYQLEKRLSVPKLF
IDTANGIGGPQLKKLLASEDWDVPAEQVEVINDRSDVPELLNFECGADYVKTNQRLPKGLSPSSFDSLYCSFDGD
ADRVVFYYVDSGSKFHLLDGDKISTLFAKFLSKOLELAHLEHSLKIGVVQTAYANGSSTAYIKNTLHCPVSCTKT
GVKHLHHEAATQYDIGIYFEANGHGTIIFSEKFHRTIKSELSKSKLNGDTLALRTLKCFSELINQTVGDAISDML
AVLATLAILKMSPMDWDEEYTDLPNKLVKCIVPDRSIFQTTDQERKLLNPVGLQDKIDLVVAKYPMGRSFVRASG
TEDAVRVYAECKDSSKLGQFCDEVVEHVKASA
SEQ ID NO: 31 is a nucleic acid sequence of promotor pTDH3
CTGCTGTAACCCGTACATGCCCAAAATAGGGGGCGGGTTACACAGAATATATAACATCGTAGGTGTCTGGGTGAA
CAGTTTATTCCTGGCATCCACTAAATATAATGGAGCCCGCTTTTTAAGCTGGCATCCAGAAAAAAAAAGAATCCC
AGCACCAAAATATTGTTTTCTTCACCAACCATCAGTTCATAGGTCCATTCTCTTAGCGCAACTACAGAGAACAGG
GGCACAAACAGGCAAAAAACGGGCACAACCTCAATGGAGTGATGCAACCTGCCTGGAGTAAATGATGACACAAGG
CAATTGACCCACGCATGTATCTATCTCATTTTCTTACACCTTCTATTACCTTCTGCTCTCTCTGATTTGGAAAAA
GCTGAAAAAAAAGGTTGAAACCAGTTCCCTGAAATTATTCCCCTACTTGACTAATAAGTATATAAAGACGGTAGG
TATTGATTGTAATTCTGTAAATCTATTTCTTAAACTTCTTAAATTCTACTTTTATAGTTAGTCTTTTTTTTAGTT
TTAAAACACCAAGAACTTAGTTTCGAATAAACACACATAAACAAACAAA
SEQ ID NO: 32 is a nucleic acid sequence of promotor pTDH3.Sk
CAATTCATCGGCCCTTTTAGCGGCTACCCGCGCCATCTAAATGATAGGGGGGTGACACTATGGTAAATCCCATA
ATTAGGTGTCTGGGTGAGTGGTTCTGATGCCGGCATCCACTAAATATATTGGAGCCCATTTTTTACGCGGGCTTC
CAGAAAAAAAGAGAATCCCAGCACCAAAAGGTGGTTCTCTTCACCAACCATCAGATCATAGGTCCACAACCACAC
ATAACAGGGGCACAAAAAGGCAAAAAACGGACATAACCTCAATGGAGTGATGCAAATTGACTGGAGCAAAAGCTG
ACACAAGGCATTGATTGACCTACGCATGTATCTGTATTCTTTTCTTACACCTTCTATTACCTTCTAACTCTTTGG
GTTGGAAAAAACTGAAAAAAAAGGTTGGGACCTGGTTCCCCCAAGTTGTCCCCCTACTTGGTTATTAAATATATA
AAGACAGCAAGTGTTGATTATAATCTTGTAAATCTATAGTTCTTAATCTATACTTCTATTTATATTTTAAATTAG
TCTTTTTATTTCCAAGTCCCCAAGAACTTAGTTTCGAATAAACACACACAAATAAACACA
SEQ ID NO: 33 is a nucleic acid sequence of promotor pTDH3-1.sba
GCAGCGCTTCTTCCGCTCTAGTTTTTATAGTTATTATTACTACCACCTTAAAAATACGTAAATACTCAAAATAGT
AGTGATATTCCCAACCTTATTCATCCAAGGCACATCATCATCATCAGCCATTCATCTTTCACCTGCCATTAGTAA
CCCGTCTTCTCATTGAGCGGGTTACGGCAGCCACAGGCCACATTCCGAATGTCTGGGTGAGCGGTCCCTTTTCCA
GCATCCACTAAATATCTCCGATCCCGCTTTTTAATCTGGCTTCCTGAAAAAAAGAGAATCCCAGCACCAAAAAAT
GGCTCTCTTCACCAACCATCAGATCATAGGTCCCATTCTCTTACCGCAACCGTACAGAACAGGGGAAAACGGGTA
CAACCTCAATGGAGTGATGCAAACTGACTGGAGCAAAAAGCTGACACAAGGCAATCGACCTACGTGTCTGTCTAT
TTTCTCACACCTTCTATTACCTTCTAACTCTCTGGGTTGGAAAAAACTGAAAAAAAGGTTGAGACCAGTTTCCAC
AAATCATCCCCCTGTTTGATTAATAAATATATAAAGACGACAACTATCGATCATAAACTCATAAAACTATAACTC
CTTTACACTTCTTATTTTATAGTTATTCTATTTTAATTCTTATTGATTTTAAAACCCCAAGAACTTAGTTTCGAA
AACACACACACACAAACAATTAAAA
SEQ ID NO: 34 is a nucleic acid sequence of promotor pTDH3.Sar
GAGCTAAATATCAGCCCTTCGGGTCCTGCCTGCTACCCGGTCCTGTTCGAATAAAAACGCGGGTAACACGACCCA
GTAACACCTGTCGTTGGGTGTCTGGGTCAGAAGTTCTGATACCGGCTTCCACTAAATAGATTGGGTTCCGCTCTT
TACGCTGGCTTCCTGAAAAAAGAGATTCCGGGCACCAAAAAATTGGTCTCTTTGCCAACCATCAGATCATAGGTC
CATTCTCTTACCATAACCACACAGGATAGGGGCACCACAGGCGAAAATGGGCACAAAATCTCAATGGAGTGATGC
AAATTAGCTGGAACAAAAGCTGACACAAGGCAATTAACCTGCGCATGTATCCATCTCCTTTTCTTACACCTTCTC
TTACGTTCTAACTGTTTGGGTTGGAAAAATTAAAAAAAAAAGGTTGAGACCAGTTTCCCCAAATCGTCCCCCTAC
TTGATTCATAAATATATAAAGACGACAACTATTGATTATAATCTTGTAAATCTATAACTCTTTACTTTCTCCTAT
TTATAATTTAACTTAATCTTTTTAGATTTAAAACCCCAAGAACTTAGTTTCGAACAAACACACACAAATAAACAA
AA
SEQ ID NO: 35 is a nucleic acid sequence of promotor pENO2
CGCTCAGCATCTGCTTCTTCCCAAAGATGAACGCGGCGTTATGTCACTAACGACGTGCACCAACTTGCGGAAAGT
GGAATCCCGTTCCAAAACTGGCATCCACTAATTGATACATCTACACACCGCACGCCTTTTTTCTGAAGCCCACTT
TCGTGGACTTTGCCATATGCAAAATTCATGAAGTGTGATACCAAGTCAGCATACACCTCACTAGGGTAGTTTCTT
TGGTTGTATTGATCATTTGGTTCATCGTGGTTCATTAATTTTTTTTCTCCATTGCTTTCTGGCTTTGATCTTACT
ATCATTTGGATTTTTGTCGAAGGTTGTAGAATTGTATGTGACAAGTGGCACCAAGCATATATAAAAAAAAAAAGC
ATTATCTTCCTACCAGAGTTGATTGTTAAAAACGTATTTATAGCAAACGCAATTGTAATTAATTCTTATTTTGTA
TCTTTTCTTCCCTTGTCTCAATCTTTTATTTTTATTTTATTTTTCTTTTCTTAGTTTCTTTCATAACACCAAGCA
ACTAATACTATAACATACAATAATA
SEQ ID NO: 36 is a nucleic acid sequence of promotor pTEF3
GGCTGATAATAGCGTATAAACAATGCATACTTTGTACGTTCAAAATACAATGCAGTAGATATATTTATGCATATT
ACATATAATACATATCACATAGGAAGCAACAGGCGCGTTGGACTTTTAATTTTCGAGGACCGCGAATCCTTACAT
CACACCCAATCCCCCACAAGTGATCCCCCACACACCATAGCTTCAAAATGTTTCTACTCCTTTTTTACTCTTCCA
GATTTTCTCGGACTCCGCGCATCGCCGTACCACTTCAAAACACCCAAGCACAGCATACTAAATTTCCCCTCTTTC
TTCCTCTAGGGTGTCGTTAATTACCCGTACTAAAGGTTTGGAAAAGAAAAAAGAGACCGCCTCGTTTCTTTTTCT
TCGTCGAAAAAGGCAATAAAAATTTTTATCACGTTTCTTTTTCTTGAAAATTTTTTTTTTTGATTTTTTTCTCTT
TCGATGACCTCCCATTGATATTTAAGTTAATAAACGGTCTTCAATTTCTCAAGTTTCAGTTTCATTTTTCTTGTT
CTATTACAACTTTTTTTACTTCTTGCTCATTAGAAAGAAAGCATAGCAATCTAATCTAAGTTTTAATTACAAA
SEQ ID NO: 37 is a nucleic acid sequence of promotor pTEF1
GTTTAGCTTGCCTCGTCCCCGCCGGGTCACCCGGCCAGCGACATGGAGGCCCAGAATACCCTCCTTGACAGTCTT
GACGTGCGCAGCTCAGGGGCATGATGTGACTGTCGCCCGTACATTTAGCCCATACATCCCCATGTATAATCATTT
GCATCCATACATTTTGATGGCCGCACGGCGCGAAGCAAAAATTACGGCTCCTCGCTGCAGACCTGCGAGCAGGGA
AACGCTCCCCTCACAGACGCGTTGAATTGTCCCCACGCCGCGCCCCTGTAGAGAAATATAAAAGGTTAGGATTTG
CCACTGAGGTTCTTCTTTCATATACTTCCTTTTAAAATCTTGCTACGATACAGTTCTCACATCACATCCGAACAT
AAACAACC
SEQ ID NO: 38 is a nucleic acid sequence of promotor pTEF1.ago
GTTTAGCTTGCCTCGTCCCCGCCGGGTCACCCGGCCAGCGACATGGAGGCCCAGAATACCCTCCTTGACAGTCTT
GACGTGCGCAGCTCAGGGGCATGATGTGACTGTCGCCCGTACATTTAGCCCATACATCCCCATGTATAATCATTT
GCATCCATACATTTTGATGGCCGCACGGCGCGAAGCAAAAATTACGGCTCCTCGCTGCAGACCTGCGAGCAGGGA
AACGCTCCCCTCACAGACGCGTTGAATTGTCCCCACGCCGCGCCCCTGTAGAGAAATATAAAAGGTTAGGATTTG
CCACTGAGGTTCTTCTTTCATATACTTCCTTTTAAAATCTTGCTACGATACAGTTCTCACATCACATCCGAACAT
AAACAACC
SEQ ID NO: 39 is a nucleic acid sequence of promotor pTEF1.Sba
CGCCAACAAACCTTCGAACACTTTAATTTTCGAGGACCGCAGATCCTCACATCACACCCACACCCAAGACTGCTT
CCCCCACACACCCTGCATCTGTACACTTTCTTCTGCTCTGTTTTTCTCTCCGGCGTTCTCTCGGGTCGCCCGCAT
CGCCGCGCCGGCTGGAACCGCCCACGCACCGCATATTGCAAATCGCCTGCCCCCTCTTGCTCCTTTTTGAGGGCG
CGCCGTTACCCGCGCCCAGGGTCCGGAAAAAGAAACAAGGCTCTACCGCGTTTCTTTTTCCTTGTCGAAAAAGGC
AAAAATGAAAATTTTTATCACGTTTCTTTTTTTTTGAAAAATTTTTTTTTTGGTTTTTTTTCTTTCGATGGCCTC
CCATTGATATTTAAGTTAATAAATGGTTTTCAGTTTTCAAGTTTCAGTTTGTGTTCTTCTTTGCTAACTTTCACT
TACACCTCGAAAGAAAGTATAGCAATCTAATCTTAGTTTTAATTACAA
SEQ ID NO: 40 is a nucleic acid sequence of promotor pPDC1
TTATTTACCTATCTCTAAACTTCAACACCTTATATCATAACTAATATTTCTTGAGATAAGCACACTGCACCCATA
CCTTCCTTAAAAACGTAGCTTCCAGTTTTTGGTGGTTCCGGCTTCCTTCCCGATTCCGCCCGCTAAACGCATATT
TTTGTTGCCTGGTGGCATTTGCAAAATGCATAACCTATGCATTTAAAAGATTATGTATGCTCTTCTGACTTTTCG
TGTGATGAGGCTCGTGGAAAAAATGAATAATTTATGAATTTGAGAACAATTTTGTGTTGTTACGGTATTTTACTA
TGGAATAATCAATCAATTGAGGATTTTATGCAAATATCGTTTGAATATTTTTCCGACCCTTTGAGTACTTTTCTT
CATAATTGCATAATATTGTCCGCTGCCCCTTTTTCTGTTAGACGGTGTCTTGATCTACTTGCTATCGTTCAACAC
CACCTTATTTTCTAACTATTTTTTTTTTAGCTCATTTGAATCAGCTTATGGTGATGGCACATTTTTGCATAAACC
TAGCTGTCCTCGTTGAACATAGGAAAAAAAAATATATAAACAAGGCTCTTTCACTCTCCTTGCAATCAGATTTGG
GTTTGTTCCCTTTATTTTCATATTTCTTGTCATATTCCTTTCTCAATTATTATTTTCTACTCATAACCTCACGCA
AAATAACACAGTCAAATCAATCAAA
SEQ ID NO: 41 is a nucleic acid sequence of promotor pCCW12
AACCAGGGCAAAGCAAAATAAAAGAAACTTAATACGTTATGCCGTAATGAAGGGCTACCAAAAACGATAATCTCA
ACTGTAAACAGGTACAATGCGGACCCTTTTGCCACAAAACATACATCATTCATTGCCGGAAAAAGAAAGAAGTGA
AGACAGCAGTGCAGCCAGCCATGTTGCGCCAATCTAATTATAGATGCTGGTGCCCTGAGGATGTATCTGGAGCCA
GCCATGGCATCATGCGCTACCGCCGGATGTAAAATCCGACACGCAAAAGAAAACCTTCGAGGTTGCGCACTTCGC
CCACCCATGAACCACACGGTTAGTCCAAAAGGGGCAGTTCAGATTCCAGATGCGGGAATTAGCTTGCTGCCACCC
TCACCTCACTAACGCTGCGGTGTGCGGATACTTCATGCTATTTATAGACGCGCGTGTCGGAATCAGCACGCGCAA
GAACCAAATGGGAAAATCGGAATGGGTCCAGAACTGCTTTGAGTGCTGGCTATTGGCGTCTGATTTCCGTTTTGG
GAATCCTTTGCCGCGCGCCCCTCTCAAAACTCCGCACAAGTCCCAGAAAGCGGGAAAGAAATAAAACGCCACCAA
AAAAAAAAATAAAAGCCAATCCTCGAAGCGTGGGTGGTAGGCCCTGGATTATCCCGTACAAGTATTTCTCAGGAG
TAAAAAAACCGTTTGTTTTGGAATTCCCCATTTCGCGGCCACCTACGCCGCTATCTTTGCAACAACTATCTGCGA
TAACTCAGCAAATTTTGCATATTCGTGTTGCAGTATTGCGATAATGGGAGTCTTACTTCCAACATAACGGCAGAA
AGAAATGTGAGAAAATTTTGCATCCTTTGCCTCCGTTCAAGTATATAAAGTCGGCATGCTTGATAATCTTTCTTT
CCATCCTACATTGTTCTAATTATTCTTATTCTCCTTTATTCTTTCCTAACATACCAAGAAATTAATCTTCTGTCA
TTCGCTTAAACACTATATCAATA
SEQ ID NO: 42 is a nucleic acid sequence of promotor pCCW12.Sm
CGTTGTGCTGTAGTGAAGGAAGACTAAAAAGGATAATCACAGTTGTAAAGAGGTATAATGCGGACCCTTTTGCCA
CAAAACACACATCTTTCGTTTCCAAAATAAGAAAGAAGAAAGCAAAAAGATTAGCAGCCACGTTGCTGCGATCTA
ATTATAGACGGTGGCGTCATCATTCTCACCCAAGATTGTGTCTTGAACCTGCCACGGGTCCTGCGTTATCGCCGG
ATGTAAAACTAGACATGCAAAAAAAGGACCTTCCAGGTAGCGTGCTCCACACCACCCATGACCACCACAGTTAGT
CCAAAAGAGGCAGCACCACTTCCCGATGGGGGAATTAGATTGCTGCCACCCTCACCTCACTAATGCTGCGGTGTG
CGGATATGCCCTGCTATATATAGCTCCGCGTTTTTGAACCAGCACAGCGCGAGCACCAAAAAGGAAAATCGCATA
GGCCCAGAACTGATTTCAGCACGGGCTATTGGCGTTGGGTTTCCGTTCTGGGAAACCTTCGCCGCGTCCCCCTCA
CAAACCTCCGCACAAGTTCGAGCAAGCGGGAAAAAACGAAAAACGCCATTAATACTAAATAAAGCAAATCCTCGA
AGCGTGGGTGGCAAGCCCCTGGATTTTTCCGCACAAGTACTCTTCTCAGGAGTAAAAAAACCCGTTTGTTTTGGA
ATTCCCCATTTCGCGGCCATCTACGCCGGTATCTTCGCAATATCTATCAGCGATAACTCAGCAATTTTAATATTC
GTGTTGCAGTGCTGCGATAGCGGGAGTCTTGTTTGTAACATAACGGCAGAAAGGAATGAGAGAAAATTTTCCATT
CTTTGGCCTCCGTTCAAGTATATAAAGCCGGCATGCTTGGTAATCTTTCTCTCTCTTCTGTATTGTTTCTATAAT
ACTTTTATCTTCTAATTATTTTCTGAAAAAACCAAGAAATTAATCTTCTGTCATTCGCTTAAACACTATATCAAT
A
SEQ ID NO: 43 is a nucleic acid sequence of promotor pCCW12.Sk
TGTAAAATCCTACACGCAAAAAACCTCTTGGGTTGCGCGTTTTCCACCACCCACGACCCGCACAATCAATCCAAA
AGGGGCAACGCCAGTTCCCAATGCGGGAATTAGCTCCTCACCCCCCTCACCCGCTAACGCTGCGGTGTGCGGACA
CGCAGCAGTATTTATAGATCCTCGTGTTGGAACCAGCCCGCGTGAGCACCAAATTGGAAAGTCGCAATGGGCCCA
GAACCGCTTTCAGTACTGGGCCATTGGCGTCTAGTTTCCGTTTTGAGCGTCCTTCGCCGCGTCTCTCTGTGAAAT
CCCCGCACAAGTCTGAGCAGGCAAAAAAAAAAAACGCCACCAAAAATAAATAAAGCCAATCCTCGAAGGATGAGT
AGGAAAGGAAGCCCCTGGTTTTTTCCCGCACGAATATTTTTCAGGAGTGAAAAAATCCGTTTGTTTTGGAATTCC
CCATTTCGCGCTCACCTACGCCGGTATCTTTGGAACAACTATCAGCGATAACTCAGCAAAATTTGCATATCCGTG
CTGCAATAGTGCGATAGTGGGATTGGGAGTCTTGTTGCATCATAACGGCAGAAAGGAATGAATAAAAATTTTCCG
TTCTTTGTCTCCGTTCAAGTATATAAAGTCGGCATGCTTGATTATCTTTTCTTCTCTTCTACTACATTTCTATTT
CACTTTCTACTCTATTCTTCCCTGAAAAACCCAAGAAAATAATCTTCTGTCATTCGCTTAAACAATATATCAAAA
SEQ ID NO: 44 is a nucleic acid sequence of promotor pCCW12.Sba
ACCATGCCACGGTGCTGGCCCCACTTCCACCCACGACCCGCACGGTTGGCCCGAAAGGGGCAACACCAGTTCCCA
ATACGGAAATTAGTCCGCCACCACCCTCACCTCGCTAGAGCTGCGGTGTGCGGGCGTGCATCGCTATTTATAGAC
ATGCCTGCTGGCGTCACCGCGCGCGAGCACCAAACAGGAAAATCGCACTGGGCCCAGAACCACGCTATGCGCTGG
GCCGATGGCGTCCGGTTTCCCTTTGGGAGCCCCCTGCCACGTTCGCCTAACAAATCCCCGCACCGGCTTGAGAAA
AAAGCGAAAAGCGAAAAAAAAAAATCAACGCCACCAAAATTAAAAAAAAAGAGCCATCCTCGAAGGGTGAATAGT
AGCCCCTGACTTTTCCCGCACAGACAGACACCTTTCAGGAGTGAACAAAAAAGCAGTTTGTTTTGGAATTCCCCC
ATTTCGCGGTGGCCTGCGCAGGTATCTCTGCGTCAACTATCAGCGATAACTCAGCAAATTTTGCATATTCGTGTT
GCGATACTACGATAATGGGAGTCTGTCGCCTAATAACGGCAACAAGGAATGAGAGAGAAAAATTTTCTTCATTCT
CCAGCTCCCGTTCAAGTATATAAGGTCGGCATGGTCGATTGTCTTTCCTTCTCTTCAGTTACGTCTCTCTATTTA
CATTATTCTTATTTTTATTTAATAAAAACCCCAAGAAATTAATCTTCTGTCATTCGCTTAAACACTATATCAAAA
SEQ ID NO: 45 is a nucleic acid sequence of promotor pCCW12.Sar
AAAAAACAACCTTCTGCCAACCTGCGTGCTTCTCACCACCCATGACCCACACAATTGACCCGAATGGGGCAACTC
CAGTTCCCAATACGGGAATTAACTCGCCACCATATTTACCGCGTTGAAGCTGTGGTGTGCGGACACTCCGTACTA
TTTATAGACCCACGCGGTGGAACCAGCACGCGCGCGCACTAAACAGGAAAATCGCATTGAGTCCAGAACCGCCAC
CAGCACTTGGCCATTGGCGTCTAATTTCCGTTTTCGGCGCCCCTCACCGCGTCCTTCTAACAAAGCGCGCACAAG
CTTGAGCAAGTGAAAAGAAAATTAAAAATAAAAAACCGCCACCAAAACAAATAAAGCAATCTTCGAAGTGTGGGT
TGGTGGGAAGCCCCTGGCTTTTCCCGCACCAGTCGTTTTCAGGAGTAAAAAAATACCCGTTTGTTTTGGAATTCC
CCATTTCGCGGCGACCTGCGCCGGTATCTTTGCAACAACTATTTGCGATAACTCAGCAAAATTTGCATATTCGTG
TTGGGATATTGCGATAGTGGGAGTCTTGTTGCATAATAACGGTAAAAAGAAGTGAAGGAAAAAAATTTGCATCCT
TTAGTCTCAGTTCAAGTATATAAAGTCGGGATATTCAATTATCTTTCTTTCTCTTGCTCAAAGGTTTCTATATTT
TTTTTATAGTATTTCTTTTGTTATAAAATACCAAGAAATTAATCTTCTGTCATTCGCTTAAATACTACATCAATA
SEQ ID NO: 46 is a nucleic acid sequence of promotor pNUP57
TCATCTGCGCAATGACTATCAAGACCTTCTGCAAGAATTTCAAATCTCACTGAAAATCTTGACCGAAAAGTGTCT
TGAAAACCCATCAAGCCTGCAAAACCTATCTTTGACATTAGTCTCCATTATAAAAACGGCATAGTTGGGAGAAAA
CTTTCCATACTTCAATTGTGGACTGATATAAGTATTTTAGTTTTGCCCGCATGATCATCCCACATGGCTACAGCA
GTTCTCTCATAGGAAAATAGTACAATAGCTACGTGATATAATCTAAATAATTGTTGCCAATGTGTAATTATATCA
TTTTGAACGTACGCGAAATGGATTATTTTCAAAAATTTTGTTTCTTGAAATGAGTAAAAGCAAAAGTCCAACTCT
CCAAGTCGATGTAAACAACTTTTTGCCAAAGGGACTGAAAGACTAAATCGAGGATTATCCCGTTCAAACTATTCC
AGAAACGCTCGTTAGTAACAAAAGACATACCTTGTTGACCAATTGATCAC
SEQ ID NO: 47 is a nucleic acid sequence of promotor pCCW10.ago
GGTACCACGGCAACCTCGTTCGCTGTTCATCCCCTTCGTCACACAGGACGTTGGATGCCGTAAGCAGCGTTGCTT
TTGATCCTCAGGATCGGCCGGGTAACCCGCGGCTGCTTCTATTTTAGTATTCATATCTCAAGCACATCCATTCCG
GCCGTTTGGGGGCGCCGCCGCACTCGTGTCCATTCCTACCGTGGCACTTAGGGCTATCCTGTCGGAGCGCCCCGC
CGACCGCCTTATCGGCACCAAAAGTAGAAGCCCCGGCCCCGCGTGGCTCAGACTCACCATCGGTGCTATTTACTT
TTCGATCAGATCGCGGCGCGCGGTGGCCGGCATTTCCGGAAGCGGCCACGGAGCAGAGGTGGCGCATTCGAATCG
CATACGTCTTCGCCACGCCGGAAAAAAAATTTTCGGCTATATAAGGAGAGGCGGCCGTCTTGCTGCAGGCAGTTT
CACTTTCTCTAAAACCAAAGAACATCGATTTCTTTAGTCACTCGCTTCCTTACACCGAACTCGAGGCGGCCGC
SEQ ID NO: 48 is a nucleic acid sequence of promotor pCWP2
CTAGCCTCCCCTTTTTATTTTGTGCGGTCACCGCAAGGGACAAAGCTTTTCTTAGAAAACCGTCTGAGAAGCATA
ACGTACGCCATCCCCTAGACATATTAATAATGCTACAGATACTATGCTGCTCGTCTTTTTTTGACGACCCTTTTA
TTGCAATGTGCAACTAATGGCAAACAACCACATAGTATCACAGTATTACATTGCCTCCACCGATGCGGATGTTAG
GGCGCCAAGTCTGTCATGAAGCATGTTCCTGTCATAATCTTGTATGCAAAATACCGCGTTCTGCGCCACTGATAT
GCTAGGCAGCAGCAACCTATGCAGAAGATTGCTTTTCCCACGCCTGTTTTACGTCTCCAGGGCACTTGAAACAAT
GCAGCGATCGCCGCCACAACACGCCAAAGAGAAGCGAAAGTGGGCCTGGGCGGCCTCAGTTTCGGCAGAGGTAAA
CAACACGAACTGAACTGCCTTAGCTCCGAAGGGCAATTCCACAGGCACTCCGCGGGGCCCGGCCAAGGCCCAAAA
GGCGTGGAATATGCGCGTTTTGGGGCCATAACACCCAGTACCACGGCCGGAACGGGCCATATAATAAGTTTTTCA
CTCTCAAGAATGGTAAACGTAAATAGGAACATCCCACTACCCTAGAAATTGCGGAAATTTCGCGCTTATCATTAG
AAAATCTGGAACCGTCCTTTTTCCTCTTTCTTGCATTTCCCTTTCCGTATTATTGCCATTCTTTAACTGCATTTG
GGGAACCGTAGACCAAAAGCCAAACAGAGAAATGTAACGTTCTAAAAAAAAAACAACGAAAAAATTGAAAAATAA
GATACAATAATCGTATATAAATCAGGCTTCTTGTTCATCATTTTCAATTCTCTTCTTGCCATCCCTTTTCCTATC
TTTGTTCTTTTCTTCTCATAATCAAGAATAAATAACTTCATCA
SEQ ID NO: 49 is a nucleic acid sequence of promotor pCCW120.Sca
TATTTTTTTTAAATTAAAGATGCTGTGCAATTAATTGTCCGGAGGGACCCTTGCATACTGGCCAGTTTGTACGGG
CGTTACATAGCGCCGATCGACGAACAGCTTATTAATAGCAAGATTTGTTTCTCCCATGAGCAATTAACCAGTAAT
TTTCACAGAAACTAGCACAAAACCAACCAAACAGTTAAAGGCAGTTTCGGAGAATAAACAACGCGCTCACTGAGC
TTGCTGTTGCCAAAAATAACAGTCGCGTGCGATGGGGGCTTGGCATTTTCCTCGAAGGGGAAACGCCGCCAGGG
TTCGCTGCGGTGTGTGGGTGGGGCAGCCCCCGGGCGCCCAGACGGTGCCGCAGGGCCTTGAGACAGCCCAAAATG
CCGCGAAAACGCAAGGGCGCGGTGACCTCTTTCCTAATGCGGAAAGCCCCACCGCCAGCGACGTGACGCGTTGTG
CGGGCTCTTCTCGGGACCCGGAAAAAGAAATACGTACACAAACACCAGCAAATTTTTTCGCTTGAGAGGCTGCCC
GTGTTTCTGCGCAACGTGAAACACGGTGGACAAGACAAGAGAGATTGACAAGCGTCGTTATCGTCCCAGTGAGGT
TTCCCAATTATATAAATACATCCCTGTTGGTGGATTCTTAATTACAACTTCCATCTCTCTCTCTCTCACTTCTTT
TGTTCCCTTACATATCCCCAAGAAATCAATCTTCTGCCATTCGCTTTAACAACAAACAAACTTTTAACTTTTAAA
SEQ ID NO: 50 is a nucleic acid sequence of promotor pRPLA1
TCAAGTTGGATACTGATCTGATCTCTCCGCCCTACTACCAGGGACCCTCATGATTACCGCTCGAATGCGACGTTT
CCTGCCTCATAAAACTGGCTTGAAAATATTTATTCGCTGAACAGTAGCCTAGCTTATAAAAATTTCATTTAATTA
ATGTAATATGAAAACTCACATGCCTTCTGTTTCTAAAATTGTCACAGCAAGAAATAACATTACCATACGTGATCT
TATTAAACTCTAGTATCTTGTCTAATACTTCATTTAAAAGAAGCCTTAACCCTGTAGCCTCATCTATGTCTGCTA
CATATCGTGAGGTACGAATATCGTAAGATGATACCACGCAACTTTGTAATGATTTTTTTTTTTTCATTTTTTAAA
GAATGCCTTTACATGGTATTTGAAAAAAATATCTTTATAAAGTTTGCGATCTCTTCTGTTCTGAATAATTTTTAG
TAAAAGAAATCAAAAGAATAAAGAAATAGTCCGCTTTGTCCAATACAACAGCTTAAACCGATTATCTCTAAAATA
ACAAGAAGAA
SEQ ID NO: 51 is a nucleic acid sequence of promotor pCUP1
CGGCAAACTTCAACGATTTCTATGATGCATTTTATAATTAGTAAGCCGATCCCATTACCGACATTTGGGCGCTAT
ACGTGCATATGTTCATGTATGTATCTGTATTTAAAACACTTTTGTATTATTTTTCCTCATATATGTGTATAGGTT
TATACGGATGATTTAATTATTACTTCACCACCCTTTATTTCAGGCTGATATCTTAGCCTTGTTACTAGTTAGAAA
AAGACATTTTTGCTGTCAGTCACTGTCAAGAGATTCTTTTGCTGGCATTTCTTCTAGAAGCAAAAAGAGCGATGC
GTCTTTTCCGCTGAACCGTTCCAGCAAAAAAGACTACCAACGCAATATGGATTGTCAGAATCATATAAAAGAGAA
GCAAATAACTCCTTGTCTTGTATCAATTGCATTATAATATCTTCTTGTTAGTGCAATATCATATAGAAGTCATCG
AAATAGATATTAAGAAAAACAAACTGTACAATCAATCAATCAATCATCACATAAA
SEQ ID NO: 52 is a nucleic acid sequence of promotor pMET6
CCACAGGAAATATTTCACGTGACTTACAAACAGAGTCGTACGTCAGGACCGGAGTCAGGTGAAAAAATGTGGGCC
GGTAAAGGGAAAAAACCAGAAACGGGACTACTATCGAACTCGTTTAGTCGCGAACGTGCAAAAGGCCAATATTTT
TCGCTAGAGTCATCGCAGTCATGGCAGCTCTTTCGCTCTATCTCCCGGTCGCAAAACTGTGGTAGTCATAGCTCG
TTCTGCTCAATTGAGAACTGTGAATGTGAATATGGAACAAATGCGATAGATGCACTAATTTAAGGGAAGCTAGCT
AGTTTTCCCAACTGCGAAAGAAAAAAAGGAAAGAAAAAAAAATTCTATATAAGTGATAGATATTTCCATCTTTAC
TAGCATTAGTTTCTCTTTTACGTATTCAATATTTTTGTTAAACTCTTCCTTTATCATAAAAAAGCAAGCATCTAA
GAGCATTGACAACACTCTAAGAAACAAAATACCAATATAATTTCAAAGTACATATCAAAA
SEQ ID NO: 53 is a nucleic acid sequence of promotor pMET25
TTACATTATCAATCCTTGCGTTTCAGCTTCCACTAATTTAGATGACTATTTCTCATCATTTGCGTCATCTTCTAA
CACCGTATATGATAATATACTAGTAACGTAAATACTAGTTAGTAGATGATAGTTGATTTTTATTCCAACACTAAG
AAATAATTTCGCCATTTCTTGAATGTATTTAAAGATATTTAATGCTATAATAGACATTTAAATCCAATTCTTCCA
ACATACAATGGGAGTTTGGCCGAGTGGTTTAAGGCGTCAGATTTAGGTGGATTTAACCTCTAAAATCTCTGATAT
CTTCGGATGCAAGGGTTCGAATCCCTTAGCTCTCATTATTTTTTGCTTTTTCTCTTGAGGTCACATGATCGCAAA
ATGGCAAATGGCACGTGAAGCTGTCGATATTGGGGAACTGTGGTGGTTGGCAAATGACTAATTAAGTTAGTCAAG
GCGCCATCCTCATGAAAACTGTGTAACATAATAACCGAAGTGTCGAAAAGGTGGCACCTTGTCCAATTGAACACG
CTCGATGAAAAAAATAAGATATATATAAGGTTAAGTAAAGCGTCTGTTAGAAAGGAAGTTTTTCCTTTTTCTTGC
TCTCTTGTCTTTTCATCTACTATTTCCTTCGTGTAATACAGGGTCGTCAGATACATAGATACAATTCTATTACCC
CCATCCATACA
SEQ ID NO: 54 is a nucleic acid sequence of promotor pSAM1
GAAACGGACGTAAGACGGAAATAGAATTTGAAGATAAAGTTATATATCACTACACACGAATACTTTCTTTTTTTT
TTTTCACAGGAAAACTGTGGTGGCGCCCTTGCCTACTAGTGCATTTCTTTTTTCGGGTTCTTGTCTCGACGAAAT
TTTAGCCTCATCGTAGTTTTTCACTCTGGTATCGATGAAAAAGGGAAGAGTAAAAAGTTTTCCGTTTAGTACTTA
ATGGGATTGGTTTGGGACGTATATATCGACTGGTGTTGTCTGTTATTCATCGTTGTTTTTCGGTTAGCTTCGAAA
AAAAAATAGAGTAAAAACCAGGAATTTACCCTAAAAACAAGAAAAAATAAGATAAACGAAAAT
SEQ ID NO: 55 is a nucleic acid sequence of terminator tTPI1
GATTAATATAATTATATAAAAATATTATCTTCTTTTCTTTATATCTAGTGTTATGTAAAATAAATTGATGACTAC
GGAAAGCTTTTTTATATTGTTTCTTTTTCATTCTGAGCCACTTAAATTTCGTGAATGTTCTTGTAAGGGACGGTA
GATTTACAAGTGATACAACAAAAAGCAAGGCGCTTTTTCTAATAAAAAGAAGAAAAGCATTTAACAATTGAACAC
CTCTATATCAACGAAGAATATTACTTTGTCTCTAAATCCTTGTAAAATGTGTACGATCTCTATATGGGTTACTCA
SEQ ID NO: 56 is a nucleic acid sequence of terminator tMET25
GTGTGCGTAATGAGTTGTAAAATTATGTATAAACCTACTTTCTCTCACAAGTACTATACTTTTATAAAACGAACT
TTATTGAAATGAATATCCTTTTTTTCCCTTGTTACATGTCGTGACTCGTACTTTGAACCTAAATTGTTCTAACAT
CAAAGAACAGTGTTAATTCGCAGTCGAGAAGAAAAATATGGTGAACAAGACTCATCTACTTCATGAGACTACTTT
ACGCCTCCTATAAAGCTGTCACACTGGATAAATTTATTGTAGGACCAAGTTACAAAAGAGGATGATGGAGGTTT
SEQ ID NO: 57 is a nucleic acid sequence of terminator tDIT1
TAAAGTAAGAGCGCTACATTGGTCTACCTTTTTGTTCTTTTACTTAAACATTAGTTAGTTCGTTTTCTTTTTCTC
ATTTTTTTATGTTTCCCCCCCAAAGTTCTGATTTTATAATATTTTATTTCACACAATTCCATTTAACAGAGGGGG
AATAGATTCTTTAGCTTAGAAAATTAGTGATCAATATATATTTGCCTTTCTTTTCATCTTTTCAGTGATATTAAT
GGTTTCGAGACACTGCAATGGCCCTAGTTGTCTAAGAGGATAGATGTTACTGTCAAAGATGATATTTTGAATTTC
SEQ ID NO: 58 is a nucleic acid sequence of terminator tRPL3
GAAGTTTTGTTAGAAAATAAATCATTTTTTAATTGAGCATTCTTATTCCTATTTTATTTAAATAGTTTTATGTAT
TGTTAGCTACATACAACAGTTTAAATCAAATTTTCTTTTTCCCAAGTCCAAAATGGAGGTTTATTTTGATGACCC
GCATGCGATTATGTTTTGAAAGTATAAGACTACATACATGTACATATATTTAAACATGTAAACCCGTCCATTATA
TTGCTTACTTTCTTCTTTTTTGCCGTTTTGACTTGGACCTCTGGTTTGCTATTTCCTTACAATCTTTGCTACAAT
SEQ ID NO: 59 is a nucleic acid sequence of terminator tRPL3.sm
GAAGTTTTTAAAGCATTTTTTAGACACTTCTCATTTTTCTAAGTTTTTTTAAAATAGTTTTATGTATTTACTACG
TATCACAATTTGAAATAATTCATCTTCCCAAAAAACTAAGATTTTTATCCTTGTCACGATCCGTAACCAGTTTAT
AATATTTTAGAGCTTATACACGTACGTATACACACGTGTCGGTACATGAGAATTACGTTCAAAATTATTCACTTT
TTTTTTCTCTGCCGTTTTACTTTTGAACTCTGTCTCGCTATTTCCTTACAATCTTCGCTACAATACCACTTGCCC
TTGG
SEQ ID NO: 60 is a nucleic acid sequence of terminator tRPL3.sba
GAAGTTTTCTGACAAAAACATAACGTTTTTTCCAATCATTTCTTATTTTTCCGGTTTATTTAAATAGTTTTTATG
TACTATTATACGTATGACTATTTAACTTAAATTCTTCCTCCCAAGAAATCTCCCAAGTTTTTCATTATCATGGCA
TACACCACTATCAGTTACAAAATGGTAGCTCAACCATATATATATCTCTATATACACATATAAATGCAAACAGGT
CCAAGTCACCGCTCACTGCAGTTTCTTTTGCCGTTTTGACTTCGATCTCTGCTTGGCTATTTTCTCACAATCCT
SEQ ID NO: 61 is a nucleic acid sequence of terminator tRPL41B
GCGGATTGAGAGCAAATCGTTAAGTTCAGGTCAAGTAAAAATTGATTTCGAAAACTAATTTCTCTTATACAATCC
TTTGATTGGACCGTCATCCTTTCGAATATAAGATTTTGTTAAGAATATTTTAGACAGAGATCTACTTTATATTTA
ATATCTAGATATTACATAATTTCCTCTCTAATAAAATATCATTAATAAAATAAAAATGAAGCGATTTGATTTTGT
GTTGTCAACTTAGTTTGCCGCTATGCCTCTTGGGTAATGCTATTATTGAATCGAAGGGCTTTATTATATTACCCT
SEQ ID NO: 62 is a nucleic acid sequence of terminator tRPL15A
GCTGGTTGATGGAAAATATAATTTTATTGGGCAAACTTTTGTTTATCTGATGTGTTTTATACTATTATCTTTTTA
ATTAATGATTCTATATACAAACCTGTATATTTTTTCTTTAACCAATTTTTTTTTTTATAGACCTAGAGCTGTACT
TTTATTCTGCTATCAAGCAAACCCCTACCCCCTCTTCTCAATCCTCCCCTCAGGCAGAACTTATCTACCTGTATC
AAGGAGCGGACGAGGGAGTCCTAATTGTTCTACGTATACCAATGCTAGCAGCTTACATAGGTGGTGGCACTACCA
SEQ ID NO: 63 is a nucleic acid sequence of terminator tRPL15A.sba
GCTGGCTGATGAAAAATATAGTTCTGTTGGGCAAGCATTTGTTTACCTAGCATCTCTTTTATACTATTATTATCT
TTATATTTGATGATTTTATATACAAGTTGTATACCTTTTCTTTAACCAATTTTTTTTTTTCTAATGGTGCACCTA
GAAGTACATTTTTTCTCACCAATAGATAGTCAAGATACTCCCAGCCTCTATGGCGTTACCACGGAGCCGACAAGG
GAAAGCTGCTTATCTTACATATGCAGATGCCAAGGCCCGTTACAGGCCGCCTTATTGATGTTTAGAAATAGCTTC
SEQ ID NO: 64 is a nucleic acid sequence of terminator tIDP1
TCGAATTTACGTAGCCCAATCTACCACTTTTTTTTTTCATTTTTTAAAGTGTTATACTTAGTTATGCTCTAGGAT
AATGAACTACTTTTTTTTTTTTTTTTTTACTGTTATCATAAATATATATACCTTATTGTTGTTTGCAACCGTCGG
TTAATTCCTTATCAAGGTTCCCCAAGTTCGGATCATTACCATCAATTTCCAACATTTTCATGAGTTCTTCTTCTT
CATTACCGTGTTTTAGGGGGCTGTTCGCACTTCTAATAGGGCTATCACCAAGCTGTTCTAATTCGTCCAAAAGTT
SEQ ID NO: 65 is a nucleic acid sequence of terminator tTEF1.sba
GGAGATTGATAGGACTTTTCTAGTTGCATATCTTTTATTTTTAAATCTTATCTATTAGTTAATTTTTTGTAATTT
ATCCTTATATATATAGTTTGGTTATTCTAAAACATCATTTCAGTATCTAAAACCTCTCTTATTCATTACCTTTTT
ATTTAATGGTTTTTGCTACAGGCAAAAATTTAATGGTTTTTGCTACAGGCAAAAATCCCGCCGTGGACTTATTCC
ACGTTAACTCGGTTACAGGGTCATGAACCATTTTGTCAATTATCGAAATAACTTCTTCAAAAGTCCCTCTTACTT
SEQ ID NO: 66 is a nucleic acid sequence of the secretion sequence added in 5′
ATGCAATTTAGCACAGTCGCATCAGTAGCCTTCGTTGCCTTGGCCAACTTCGTGGCAGCA
SEQ ID NO: 67 is an amino acid sequence of the secretion sequence added in N-ter
MQFSTVASVAFVALANFVAA
SEQ ID NO: 68 is a nucleic acid sequence of the anchoring sequence added in 3'
GGATCCGCCATTTCTCAAATCACTGACGGTCAAATCCAAGCTACTACCACTGCTACCACCGAAGCTACCACCACT
GCTGCCCCATCTTCCACCGTTGAAACTGTTTCTCCATCCAGCACCGAAACTATCTCTCAACAAACTGAAAATGGT
GCTGCTAAGGCCGCTGTCGGTATGGGTGCCGGTGCTCTAGCTGCTGCTGCTATGTTGTTATAA
SEQ ID NO: 69 is an amino acid sequence of the anchoring sequence added in
C-ter AISQITDGQIQATTTATTEATTTAAPSSTVETVSPSSTETISQQTENGAAKAAVGMGAGALAAAAMLL

1. A recombinant host cell (i) or a recombinant yeast cell (ii) producing heparosan wherein the recombinant host cell (i) and the recombinant yeast cell (ii) comprise:
(a) one or more recombinant nucleic acids encoding a polypeptide having a heparosan synthase (HSS) activity;
(b) one or more recombinant nucleic acids encoding a polypeptide having a UDP-Glucose dehydrogenase (UDP-GlcDH or HASB) activity,
wherein:
the recombinant host cell (i) further comprises (c) one or more recombinant nucleic acids encoding a polypeptide having a glucuronosyl-disulfoglucosamine glucuronidase activity, and
the recombinant yeast cell (ii) optionally further comprises (c) one or more recombinant nucleic acids encoding a polypeptide having a glucuronosyl-disulfoglucosamine glucuronidase activity,
wherein the polypeptide having a glucuronosyl-disulfoglucosamine glucuronidase activity comprises a secretion signal, and optionally an anchoring signal, heparosan is produced by the recombinant host cell (i) or the recombinant yeast cell (ii).

2. (canceled)

3. A The recombinant host cell (i) or the recombinant yeast cell (ii) according to claim 1,
wherein the heparosan is of a desired molecular weight.

4. The recombinant host cell (i) or the recombinant yeast cell (ii) according to claim 1, wherein the nucleic acid encoding a polypeptide having glucuronosyl-disulfoglucosamine glucuronidase activity is obtained or derived from Pedobacter heparinus.

5. The recombinant host cell (i) or the recombinant yeast cell (ii) according to claim 1, wherein the nucleic acid encoding a polypeptide having a glucuronosyl-disulfoglucosamine glucuronidase activity is under the control of a promoter selected from the group consisting of pTEF1, pCCW12, pCCW12.sba, pCCW12.Sar, pPDC1, pTEF3, pTDH3, pNUP57 and pCCW10.ago.

6. The recombinant host cell (i) or the recombinant yeast cell (ii) according to claim 1, wherein the heparosan has a molecular weight that is less than 50 kDa, or is in a range of 20 kDa to 50 kDa.

7. The recombinant host cell (i) or the recombinant yeast cell (ii) according to claim 1, wherein the heparosan has a molecular weight that is greater than 50 kDa, or is in a range of 50 kDa to 250 kD, or is greater than 100 kDa or is in a range of 100 kDa to 1500 kDa.

8. (canceled)

9. The recombinant host cell (i) or the recombinant yeast cell (ii) according to claim 1, wherein the one or more recombinant nucleic acids encoding a polypeptide having a UDP-Glucose dehydrogenase activity is/are obtained or derived from at least one of Arabidopsis thaliana, Chlorella virus PBCV1 and Streptococcus zooepidemicus.

10. The recombinant host cell (i) or the recombinant yeast cell (ii) according to claim 1, wherein the one or more recombinant nucleic acids encoding a polypeptide having a heparosan synthase (HSS) activity is/are obtained or derived from Pasteurella multocida.

11. (canceled)

12. The recombinant host cell (i) or the recombinant yeast cell (ii) according to claim 1, further comprising at least one recombinant nucleic acid encoding one or more of:
(i) a polypeptide having a Phosphoglucomutase-1 (PGM1) activity; and/or
(ii) a polypeptide having a UTP-glucose-1-phosphate uridylyltransferase (UGP1) activity; and/or
(iii) a polypeptide having a Glucosamine-6-phosphate N-acetyltransferase (GNA1) activity; and/or
(iv) a polypeptide having a phosphoacetylglucosamine mutase (PCM1) activity; and/or
(v) a polypeptide having a glutamine-fructose-6-phosphate amidotransferase (GFA1) activity; and/or
(vi) a polypeptide having a UDP-N-acetylglucosamine pyrophosphorylase (QRI1) activity.

13. The recombinant host cell (i) or the recombinant yeast cell (ii) according to claim 12, wherein:
the nucleic acid encoding the polypeptide having glutamine-fructose-6-phosphate amidotransferase (GFA1) activity; and/or
the nucleic acid encoding the polypeptide having UDP-N-acetylglucosamine pyrophosphorylase (QRI1) activity; and/or
the nucleic acid encoding the polypeptide having Phosphoglucomutase-1 (PGM1) activity; and/or
the nucleic acid encoding the polypeptide having UTP-glucose-1-phosphate uridylyltransferase (UGP1) activity; and/or
the nucleic acid encoding the polypeptide having Glucosamine-6-phosphate N-acetyltransferase (GNA1) activity; and/or
the nucleic acid encoding the polypeptide having phosphoacetylglucosamine mutase (PCM1);
are obtained or derived from Saccharomyces cerevisiae.

14. (canceled)

15. The recombinant host cell (i) or the recombinant yeast cell (ii) according to claim 1, wherein the recombinant yeast cell (ii) belongs to a genus selected from the group consisting of Saccharomyces, Candida, Kluyveromyces, Ogataea, Yarrowia, Debaryomyces and Ashbya.

16. The recombinant host cell (i) or the recombinant yeast cell (ii) according to claim 15, wherein the recombinant yeast cell (ii) is selected from the group consisting of Saccharomyces cerevisiae, Saccharomyces boulardii, Saccharomyces bayanus, Saccharomyces paradoxus, Saccharomyces mikatae, Saccharomyces castelli, Candida albicans, Candida glabrata, Candida tropicalis, Kluyveromyces lactis, Kluyveromyces marxianus, Kluyveromyces polysporus, Kluyveromyces thermotolerens, Ogataea polymorpha, Yarrowia lypolytica, Debaryomyces hansenii, and Ashbya gossypii, and is preferably Saccharomyces cerevisiae.

17. A method of producing heparosan comprising:
(a) cultivating the recombinant host cell (i) or the recombinant yeast cell (ii) of claim 1 in a cultivation medium for a time sufficient to produce heparosan; and
(b) optionally isolating or recovering the heparosan from the recombinant cell and/or from the cultivation medium.

18. The method according to claim 17, wherein the heparosan has a molecular weight of from 20 kDa to 50 kDa or wherein the heparosan has a molecular weight of from 50 kDa to 150 kDa or wherein the heparosan has a molecular weight of from 150 kDa to 1500 kDa.

19. (canceled)

20. (canceled)

21. The method according to claim 17, wherein the yeast recombinant cell (ii) is a yeast belonging to the Saccharomyces genus.

22. The method according to claim 17, wherein the time sufficient to produce heparosan is a period of from 35 to 50 hours, or from 40 to 50 hours, or 48 hours.

23. The method according to claim 17, wherein the molecular weight of the produced heparosan is controlled by regulating the pH of the cultivation medium during step (a).

24. The method according to claim 17, wherein the method is carried out on an industrial scale, where the cultivation medium is at least 100 L, or is in the range of 1000 L to 3000 L, or is 10,000 L or is 100,000 L, or is 250,000 L.

25. Heparosan obtainable from the recombinant host cell (i) or the recombinant yeast cell (ii) according to claim 1.

26. A cultivation medium comprising the heparosan according to claim 25.

27. A composition comprising the heparosan according to claim 25.

28. An industrial product or a consumer product or a consumable comprising (i) the heparosan according to claim 25, (ii) a cultivation medium comprising the heparosan or (iii) a composition comprising the heparosan.

29. The industrial product or the consumer product or the consumable of claim 28, wherein the industrial product or the consumer product or the consumable is a cosmetic product, a flavour product, a fragrance product, a food product, a food, a beverage, a texturant, a pharmaceutical composition, a dietary supplement, a nutraceutical, a cleaning product, a dental and/or an oral hygiene composition.

30. (canceled)


SEQUENCE LISTING (69 sequences)

SEQ ID NO: 1 - DNA, 1854 bp, Pasteurella multocida
  reencoded nucleic acid sequence of heparosan synthase originating from Pasteurella multocida HSS1
atgagtttgttcaaaagggcgactgagctgttcaaatcgggtaactataaggatgcactgacattgtacgagaacatagccaaaatttatggctcagaatccctagtgaagtataacatagatatctgcaagaagaacataacccaatcaaaatcgaacaagatagaagaggacaatatttccggagagaataagttctccgtctctatcaaagacttatacaacgaaattagtaattctgagctagggataacaaaggagagattaggagcaccacccttggtaagcataattatgacgagtcataatacagagaaattcatcgaagcttccatcaacagtttattattgcaaacttataacaatcttgaggtcattgtggtagacgactactccacagacaagaccttccaaatcgcgtccaggattgcaaactcgacgtcaaaagttaagactttcagattgaattccaaccttggaacgtacttcgcaaagaatacgggaatcctaaaatcaaaaggggacattatattctttcaagattctgatgatgtctgccatcatgaacgtattgagagatgtgtaaatgcacttttgtctaacaaagataacattgccgttaggtgcgcttattctcgtattaatctggagacccagaacatcataaaggtcaacgacaacaagtacaagttaggtcttattaccctaggagtataccgtaaagtatttaatgaaataggattcttcaactgtaccactaaggcaagtgacgacgaattttatcacaggatcatcaaatactatggcaagaataggataaacaatttgttcctacctctatactacaataccatgcgtgaagatagtttgtttagcgatatggtcgagtgggtagacgaaaacaacattaaacagaaaacaagtgatgcacgtcagaattatctgcatgaattccaaaagatccacaacgaaaggaaattgaatgagttaaaggagattttcagcttccccaggattcacgatgccctaccgataagtaaagagatgagcaagttgtctaacccaaagatccctgtctacattaacatttgcagtataccatctaggataaagcaattacagtatacgatcggagtgctgaagaatcagtgtgaccatttccacatttacttagatggttatccagaggttccagatttcataaagaagctaggcaacaaggctaccgtaatcaattgccaaaacaagaacgagtctatcagggacaacggcaagtttatccttttagaaaagctgatcaaagagaataaggacggctattatattacctgtgatgatgacataaggtatcctgctgactacacgaataccatgataaagaagattaacaagtacaatgacaaagcggcaataggtctacacggcgtcatctttccgtccagagtgaataaatacttctcatctgacaggattgtttataactttcagaagcccttggaaaacgacacagccgtcaacattttgggtactgggaccgttgcatttagagtctccatcttcaacaagttttccctgagcgacttcgagcaccctggtatggtggatatttatttctcaatactatgcaagaagaacaacatattgcaagtttgtataagcaggccctcaaactggttaaccgaggacaacaagaataccgaaaccctgttccacgagttccaaaaccgtgacgagatccagagtaagttgatcatctctaataatccgtggggctacagtagcatctaccccttacttaacaacaatgccaactatagcgagttgataccatgcttatcattctataatgagtaa

SEQ ID NO: 2 - DNA, 1956 bp, Pasteurella multocida
  reencoded nucleic acid sequence of heparosan synthase originating from Pasteurella multocida (HSS2)
atgaagggcaagaaagagatgacccaaatacagattgctaagaacccgccccaacacgagaaggaaaacgagctgaatactttccaaaacaagatcgactcccttaaaacgacattaaacaaggacattatatcacaacagacattgttggctaaacaggactcaaagcacccattatctgcaagtcttgagaacgagaacaaacttcttcttaaacagttgcagcttgttttgcaggaatttgaaaagatatacacttacaaccaagcactagaggctaagcttgagaaagacaaacagaccacctcaataaccgatttatataacgaagttgcaaagtctgatctagggctagttaaggaaactaacagtgctaaccctctagtctccattattatgacttcgcacaataccgctcaatttatcgaggcatcgattaactccttgttgctacagacatataagaacattgagataattatagtcgatgatgacagttccgacaacacttttgaaatcgcctcaaggatcgcaaacacaacgtctaaggtccgtgtgtttcgtttgaactccaatctgggcacgtactttgctaagaatacaggaatccttaagagtaaaggcgacataatattcttccaggactccgacgacgtttgtcatcatgagagaatcgaaaggtgcgtgaacattttgttggctaacaaagagacgattgctgtgaggtgcgcatacagtaggctggctcctgaaacgcagcatataattaaagtcaacaacatggattataggcttggtttcatcacactgggaatgcataggaaggttttccaagagattgggttcttcaactgtacaacaaaaggcagtgacgacgagttcttccaccgtatagctaaatactacggtaaagaaaagattaagaaccttttattgccgctttattataacacgatgcgtgaaaactcacttttcactgacatggtcgagtggatcgacaaccataatatcatacagaagatgtctgacacacgtcaacactatgctactctgtttcaggcgatgcataatgagactgcatcacacgacttcaagaacttgttccagttcccacgtatatacgacgccttgcctgttccacaggagatgtctaagttatctaaccctaaaataccggtgtacataaatatatgcagtataccatctaggattgcacagttacgtaggattattgggatcttaaagaaccagtgcgaccactttcacatatacctagacggatacgtggagatccctgattttattaagaatcttggtaataaagcaacggtcgtccactgcaaagacaaggacaactctattagggacaacggcaagtttattctactggaagagttaatagaaaagaatcaagacgggtactacataacgtgcgacgacgacattatctatccatcggactatataaacacaatgattaagaagctgaacgagtatgatgacaaggcggtcatagggcttcacggaatattatttccgtcaaggatgacgaaatacttttccgccgataggcttgtttattcgttttacaaacccctagagaaagacaaagcagtaaatgttcttgggaccggaactgtctcattcagagttagtctgttcaaccaattctccctttcagacttcacccattcgggaatggccgacatctacttctctctattgtgcaagaagaacaacattttacaaatctgtatctccagaccggcaaattggttgacggaagataacagagattctgagaccttataccaccagtatagagataatgacgagcagcagactcagttgatcatggaaaatgggccctggggttactcaagtatctatccacttgtcaagaatcacccgaagtttacggacttaataccatgtttgcctttctacttcctataa

SEQ ID NO: 3 - protein, 617 aa, Pasteurella multocida
  amino acid sequence of heparosan synthase originating from Pasteurella multocida (HSS1)
MSLFKRATELFKSGNYKDALTLYENIAKIYGSESLVKYNIDICKKNITQSKSNKIEEDNISGENKFSVSIKDLYNEISNSELGITKERLGAPPLVSIIMTSHNTEKFIEASINSLLLQTYNNLEVIVVDDYSTDKTFQIASRIANSTSKVKTFRLNSNLGTYFAKNTGILKSKGDIIFFQDSDDVCHHERIERCVNALLSNKDNIAVRCAYSRINLETQNIIKVNDNKYKLGLITLGVYRKVFNEIGFFNCTTKASDDEFYHRIIKYYGKNRINNLFLPLYYNTMREDSLFSDMVEWVDENNIKQKTSDARQNYLHEFQKIHNERKLNELKEIFSFPRIHDALPISKEMSKLSNPKIPVYINICSIPSRIKQLQYTIGVLKNQCDHFHIYLDGYPEVPDFIKKLGNKATVINCQNKNESIRDNGKFILLEKLIKENKDGYYITCDDDIRYPADYTNTMIKKINKYNDKAAIGLHGVIFPSRVNKYFSSDRIVYNFQKPLENDTAVNILGTGTVAFRVSIFNKFSLSDFEHPGMVDIYFSILCKKNNILQVCISRPSNWLTEDNKNTETLFHEFQNRDEIQSKLIISNNPWGYSSIYPLLNNNANYSELIPCLSFYNE

SEQ ID NO: 4 - protein, 652 aa, Pasteurella multocida
  amino acid sequence of heparosan synthase originating from Pasteurella multocida (HSS2)
MAKGKKEMTQIQIAKNPPQHEKENELNTFQNKIDSLKTTLNKDIISQQTLLAKQDSKHPLSASLENENKLLLKQLQLVLQEFEKIYTYNQALEAKLEKDKQTTSITDLYNEVAKSDLGLVKETNSANPLVSIIMTSHNTAQFIEASINSLLLQTYKNIEIIIVDDDSSDNTFEIASRIANTTSKVRVFRLNSNLGTYFAKNTGILKSKGDIIFFQDSDDVCHHERIERCVNILLANKETIAVRCAYSRLAPETQHIIKVNNMDYRLGFITLGMHRKVFQEIGFFNCTTKGSDDEFFHRIAKYYGKEKIKNLLLPLYYNTMRENSLFTDMVEWIDNHNIIQKMSDTRQHYATLFQAMHNETASHDFKNLFQFPRIYDALPVPQEMSKLSNPKIPVYINICSIPSRIAQLRRIIGILKNQCDHFHIYLDGYVEIPDFIKNLGNKATVVHCKDKDNSIRDNGKFILLEELIEKNQDGYYITCDDDIIYPSDYINTMIKKLNEYDDKAVIGLHGILFPSRMTKYFSADRLVYSFYKPLEKDKAVNVLGTGTVSFRVSLFNQFSLSDFTHSGMADIYFSLLCKKNNILQICISRPANWLTEDNRDSETLYHQYRDNDEQQTQLIMENGPWGYSSIYPLVKNHPKFTDLIPCLPFYFL

SEQ ID NO: 5 - DNA, 1446 bp, Arabidopsis thaliana
  nucleic acid sequence of UDP-Glucose dehydrogenase (HASB) originating from Arabidopsis thaliana
atggtcaaaatatgctgtatcggggctggatatgtcggtgggcctacaatggccgttatggccctaaagtgtccagagattgaagtcgtggtagttgatatatcggagccacgaattaacgcatggaactcagaccgtctaccaatttatgagccagggttagaggatgtcgtcaaacagtgtagagggaagaatttgttcttctctacagatgtagagaagcatgtatttgagtcagatatagtgtttgtttcggtaaacactcctacgaaaacgcagggtctgggtgcaggtaaagcagcagatttgacatattgggagtccgctgctcgcatgatagctgatgtgagcaaatcgtcgaaaatcgtcgttgaaaagagtacagtaccagttcgtacagccgaggctatagaaaagattttaacgcacaattcgaagggtatcgaattccagatcttatcaaatccagaattcttggccgaagggacggcgattaaagacttatataaccctgatagagttctaatcggtggcagggacaccgctgcgggacaaaaggccattaaggcgttgcgtgacgtgtatgcccactgggttcctgttgagcaaataatctgtactaatttatggagtgccgagctatcaaagttggctgcgaacgcatttctagctcaaaggataagttcagtaaacgcaatgtcagcgctttgtgaggcaactggtgctgacgtaacccaagtcgctcacgccgttggaaccgacactagaattggaccgaagtttcttaacgcatccgtaggcttcggcggatcttgctttcagaaagacatcctgaatcttatttacatctgcgaatgcaatggtcttccagaagcagccaattattggaaacaggtagtcaaggtaaatgactaccaaaagattaggtttgctaatcgagtcgtatcttctatgttcaacaccgtctccggtaagaaaattgctattttgggatttgcgttcaagaaggacaccggcgacacgcgtgaaactcctgccatagatgtgtgtaatcgcctcgtggctgataaagcaaagctgtcgatctatgatccgcaagtattagaagagcagatccgccgtgatctgtccatggcccgattcgattgggaccatccagtcccactccagcagatcaaagctgaaggtatctccgaacaggttaacgttgtgtccgacgcctacgaggctacgaaggatgctcatggtttatgtgttttaaccgaatgggacgaattcaagtcacttgattttaagaagatctttgataatatgcagaaacccgctttcgttttcgacggaagaaacgtggtcgacgctgtgaaattgagagaaattggattcatagtatattccataggtaaacctctggatagttggctcaaggatatgccggctgttgcataa

SEQ ID NO: 6 - DNA, 1166 bp, Chlorella virus
  reencoded nucleic acid sequence of UDP-Glucose dehydrogenase (HASB) originating from Chlorella virus PBCV-1
atgagtagaattgctgtcgttggatgcggttacgtgggtacggcctgcgccgtacttttggcgcagaagaacgaagttatcgttttggatatctcggaggaccgggtacaactgattaagaataagaagtcacctatagaagataaggaaatcgaagaattcctggagacgaaagatttgaatctaacagcgacgacggataaggtgctcgcctatgagaatgctgaattcgttataatagctacaccgaccgattacgatgtcgtcactagatatttcaacactaagtctgttgaaaatgtgataggcgatgtcattaagaacactcagacacaccctactatagtgatcaagagtactattccaatcggtttcgtggataaagttagagaacagttcgattatcaaaatattatcttctcgccggaattcttgagagaaggacgagcattatatgataatctttacccctcccgtatcatcgtcggtgatgactccccaattgccttaaaattcgcgaatctcttggtcgagggtagcaaaactccactagctcccgtattaactatgggtacgcgagaggccgaagctgtaaaactattttcaaatacatatttggctatgagagtagcatacttcaatgagctagacacatttgcaatgtcccatggtatgaacgcaaaagagattatcgacggagtcacactagaaccaaggatagggcagggttattcgaatccttcatttgggtatggagcctattgtttcccaaaagatacaaagcaattgttggctaattttgaaggggttccacaagatattattggcgcaatcgtagagtctaacgaaactagaaaggaggttattgtttctgaggtcgagaacagatttccgaccacagttggcgtatataagttggctgctaaagctggttcggataatttcaggtctagtgcgatagtcgatatcatggaaaggttggctaataagggctaccacatcaaaatatttgaacctaccgttgaacaatttgagaattttgaggtagataataatctcacaacttttgcaaccgagtctgatgtaataattgcgaacagagttccagtcgaacatcgcattctgtttgggaagaagttaataacacgggatgtctatggggataa

SEQ ID NO: 7 - DNA, 1170 bp, Chlorella virus
  reencoded nucleic acid sequence of UDP-Glucose dehydrogenase (HASB-A) originating from Chlorella virus PBCV-1
atgtcacgaatcgcagtagttggctgtgggtacgtgggaaccgcatgcgctgtactgctggcgcaaaagaatgaggtaatcgttcttgatattagtgaggatagggtccaactaatcaagaacaagaaatccccgatcgaagataaggagattgaagaattcttggaaacaaaagacctaaacttaactgcaacaactgacaaagttttagcctacgaaaacgctgagtttgtgattatagcgacacccacagattatgacgtagttaccagatactttaacacgaagtccgtagagaacgtcattggagatgttataaagaatactcagactcatcctacgatagtaataaagtcaaccattcccataggtttcgtagataaggttagggagcaattcgattaccagaacattatattttcgccagaatttctgagagagggtcgcgccctgtatgataatctatatccatcacggattatagtgggcgatgactctccgattgcacttaagtttgctaatcttttagttgagggctccaaaactccgctcgccccagtacttacgatgggtacacgtgaggctgaagctgtcaagctgttttcaaacacataccttgctatgcgagtcgcatactttaacgaactagatacctttgctatgtcgcacggtatgaatgctaaagaaatcatagatggcgtaacgttggagcctcggataggtcaaggatattccaatccatcttttggctacggtgcgtattgtttcccaaaggacacgaagcaattattagctaactttgagggtgttccgcaagatataattggggcgatagtagaaagcaatgaaacacggaaggaggtaatcgtgagtgaagtggaaaaccgattccccactacggtcggcgtttacaaattagccgccaaggctggttccgacaatttccgatccagcgcaatagtagatattatggaaagattagctaataagggataccacattaaaatctttgaacctactgtcgaacagttcgagaacttcgaggttgataataacttgacgactttcgcaacggagagcgatgtaattattgcaaaccgcgtacctgtggaacatcgaattttgttcggaaagaagctgattacacgcgatgtatatggcgataactaa

SEQ ID NO: 8 - protein, 481 aa, Arabidopsis thaliana
  amino acid sequence of UDP-Glucose dehydrogenase (HASB) originating from Arabidopsis thaliana
MVKICCIGAGYVGGPTMAVMALKCPEIEVVVVDISEPRINAWNSDRLPIYEPGLEDVVKQCRGKNLFFSTDVEKHVFESDIVFVSVNTPTKTQGLGAGKAADLTYWESAARMIADVSKSSKIVVEKSTVPVRTAEAIEKILTHNSKGIEFQILSNPEFLAEGTAIKDLYNPDRVLIGGRDTAAGQKAIKALRDVYAHWVPVEQIICTNLWSAELSKLAANAFLAQRISSVNAMSALCEATGADVTQVAHAVGTDTRIGPKFLNASVGFGGSCFQKDILNLIYICECNGLPEAANYWKQVVKVNDYQKIRFANRVVSSMFNTVSGKKIAILGFAFKKDTGDTRETPAIDVCNRLVADKAKLSIYDPQVLEEQIRRDLSMARFDWDHPVPLQQIKAEGISEQVNVVSDAYEATKDAHGLCVLTEWDEFKSLDFKKIFDNMQKPAFVFDGRNVVDAVKLREIGFIVYSIGKPLDSWLKDMPAVA

SEQ ID NO: 9 - protein, 389 aa, Chlorella virus
  amino acid sequence of UDP-Glucose dehydrogenase (HASB) originating from Chlorella virus PBCV1
MSRIAVVGCGYVGTACAVLLAQKNEVIVLDISEDRVQLIKNKKSPIEDKEIEEFLETKDLNLTATTDKVLAYENAEFVIIATPTDYDVVTRYFNTKSVENVIGDVIKNTQTHPTIVIKSTIPIGFVDKVREQFDYQNIIFSPEFLREGRALYDNLYPSRIIVGDDSPIALKFANLLVEGSKTPLAPVLTMGTREAEAVKLFSNTYLAMRVAYFNELDTFAMSHGMNAKEIIDGVTLEPRIGQGYSNPSFGYGAYCFPKDTKQLLANFEGVPQDIIGAIVESNETRKEVIVSEVENRFPTTVGVYKLAAKAGSDNFRSSAIVDIMERLANKGYHIKIFEPTVEQFENFEVDNNLTTFATESDVIIANRVPVEHRILFGKKLITRDVYGDN

SEQ ID NO: 10 - DNA, 1968 bp, Pedobacter heparinus
  reencoded nucleic acid sequence of glucuronosyl-disulfoglucosamine glucuronidase originating from Pedobacter heparinus with a N-terminal secretion signal
atgcaattctcgacggtcgcaagtgttgcctttgttgctttggctaacttcgttgccgcacagtcctcgtcaatcaccaggaaggacttcgatcatatcaacttggaatactccggtttggagaaggtgaacaaggcggtagcagcaggcaattacgacgacgcggcaaaagccctactagcttactaccgtgaaaagagcaaagcaagagagcctgactttagtaatgccgagaagcctgccgacatacgtcagcctattgacaaggtaacacgtgagatggcggataaagcattagtacatcagtttcaacctcacaaaggatacggatattttgattatgggaaggatattaattggcagatgtggccagttaaagacaacgaggtcagatggcagttgcatagggtaaaatggtggcaggcaatggccttggtttaccatgccacgggcgacgagaagtacgccagggagtgggtgtaccaatactcggactgggcgaggaagaacccgttaggcttatcgcaagacaatgataagtttgtctggagacctttggaagtctcagatagagtccagtcattgccacctaccttttctcttttcgtcaacagtcccgccttcacaccagcattcctaatggagttcttaaactcgtaccaccaacaggcagactacttgtcaacacactacgccgagcaggggaaccataggctgttcgaagctcagaggaacttgttcgccggcgtatccttccccgaattcaaggactcacccaggtggagacagactggaatatctgttctgaatacagagataaagaagcaggtgtacgccgatggcatgcaattcgagttaagtccgatataccacgttgctgccatagatatctttcttaaagcatacggaagcgccaagagagttaatttggagaaagagttccctcaatcctatgtgcaaaccgtcgagaatatgattatggcgttaatttcaataagtctaccggactataatacgcccatgttcggcgactcttggatcactgataagaatttcagaatggctcaatttgcctcatgggcgcgtgtcttccccgctaaccaggcaattaagtactttgcaaccgacggaaaacagggtaaagctcccaacttcttgagtaaggcattgtccaatgcaggattctacacgtttagaagtggatgggataagaacgcgacagtgatggtactgaaggcaagtccgcctggtgagtttcacgctcagcctgataacggaacttttgaactgttcatcaagggtaggaacttcacacccgacgccggtgtattcgtctattccggtgacgaggcgattatgaaattgaggaactggtacaggcagacaaggattcactctactctaactcttgataatcagaatatggtcataacgaaagcacgtcagaataaatgggagacaggcaataacttggatgtcttaacttacactaacccgtcataccccaacttagatcaccaacgttcagttctattcattaacaagaagtatttcttagtcatagatagggctatcggcgaggccacgggcaaccttggcgtgcactggcagctaaaggaagacagtaaccctgtcttcgataaaaccaagaatagagtctatactacctatagggacgggaacaatctaatgatacagagtctaaacgcagacaggacatcactgaacgaggaagaagggaaggtatcttatgtctataacaaggagctgaagcgtccagctttcgtatttgagaagcctaagaagaacgcagggacccaaaactttgtgtcaattgtctatccatacgacggtcagaaggcgccggaaatttctattagagaaaataagggaaatgacttcgaaaagggaaaattgaacctaacactgactataaacggtaagcaacagcttgtgttggttccttaa

SEQ ID NO: 11 - DNA, 2178 bp, Pedobacter heparinus
  reencoded nucleic acid sequence of glucuronosyl-disulfoglucosamine glucuronidase originating from Pedobacter heparinus with a N-terminal secretion signal and a C-terminal anchoring signal
atgcaattctcgacggtcgcaagtgttgcctttgttgctttggctaacttcgttgccgcacagtcctcgtcaatcaccaggaaggacttcgatcatatcaacttggaatactccggtttggagaaggtgaacaaggcggtagcagcaggcaattacgacgacgcggcaaaagccctactagcttactaccgtgaaaagagcaaagcaagagagcctgactttagtaatgccgagaagcctgccgacatacgtcagcctattgacaaggtaacacgtgagatggcggataaagcattagtacatcagtttcaacctcacaaaggatacggatattttgattatgggaaggatattaattggcagatgtggccagttaaagacaacgaggtcagatggcagttgcatagggtaaaatggtggcaggcaatggccttggtttaccatgccacgggcgacgagaagtacgccagggagtgggtgtaccaatactcggactgggcgaggaagaacccgttaggcttatcgcaagacaatgataagtttgtctggagacctttggaagtctcagatagagtccagtcattgccacctaccttttctcttttcgtcaacagtcccgccttcacaccagcattcctaatggagttcttaaactcgtaccaccaacaggcagactacttgtcaacacactacgccgagcaggggaaccataggctgttcgaagctcagaggaacttgttcgccggcgtatccttccccgaattcaaggactcacccaggtggagacagactggaatatctgttctgaatacagagataaagaagcaggtgtacgccgatggcatgcaattcgagttaagtccgatataccacgttgctgccatagatatctttcttaaagcatacggaagcgccaagagagttaatttggagaaagagttccctcaatcctatgtgcaaaccgtcgagaatatgattatggcgttaatttcaataagtctaccggactataatacgcccatgttcggcgactcttggatcactgataagaatttcagaatggctcaatttgcctcatgggcgcgtgtcttccccgctaaccaggcaattaagtactttgcaaccgacggaaaacagggtaaagctcccaacttcttgagtaaggcattgtccaatgcaggattctacacgtttagaagtggatgggataagaacgcgacagtgatggtactgaaggcaagtccgcctggtgagtttcacgctcagcctgataacggaacttttgaactgttcatcaagggtaggaacttcacacccgacgccggtgtattcgtctattccggtgacgaggcgattatgaaattgaggaactggtacaggcagacaaggattcactctactctaactcttgataatcagaatatggtcataacgaaagcacgtcagaataaatgggagacaggcaataacttggatgtcttaacttacactaacccgtcataccccaacttagatcaccaacgttcagttctattcattaacaagaagtatttcttagtcatagatagggctatcggcgaggccacgggcaaccttggcgtgcactggcagctaaaggaagacagtaaccctgtcttcgataaaaccaagaatagagtctatactacctatagggacgggaacaatctaatgatacagagtctaaacgcagacaggacatcactgaacgaggaagaagggaaggtatcttatgtctataacaaggagctgaagcgtccagctttcgtatttgagaagcctaagaagaacgcagggacccaaaactttgtgtcaattgtctatccatacgacggtcagaaggcgccggaaatttctattagagaaaataagggaaatgacttcgaaaagggaaaattgaacctaacactgactataaacggtaagcaacagcttgtgttggttcctggatccgccatttctcaaatcactgacggtcaaatccaagctactaccactgctaccaccgaagctaccaccactgctgccccatcttccaccgttgaaactgtttctccatccagcaccgaaactatctctcaacaaactgaaaatggtgctgctaaggccgctgtcggtatgggtgccggtgctctagctgctgctgctatgttgttataa

SEQ ID NO: 12 - DNA, 1968 bp, Pedobacter heparinus
  reencoded nucleic acid sequence of glucuronosyl-disulfoglucosamine glucuronidase originating from Pedobacter heparinus with a N-terminal secretion sigal
atgcagttttcaaccgtggcatctgtggcattcgtggcattggcaaattttgtcgctgctcaatcttccagtattacgcgtaaagattttgaccacataaatcttgagtatagtggcctagagaaagtcaataaagctgtcgccgctggtaactatgatgatgcagccaaggctttgttagcctattatagagagaaatctaaggctcgtgaaccggatttctctaacgctgaaaagccagcggatatcagacaaccaatcgataaagtcaccagggaaatggcagacaaggctttggttcaccaattccagccccataagggctatggttacttcgactacggtaaagacataaactggcaaatgtggcccgtaaaggataatgaagtaaggtggcaacttcacagagtgaagtggtggcaagctatggctctggtgtatcacgcaaccggtgatgaaaagtatgctagagaatgggtatatcagtattctgattgggctaggaagaatccactaggactttctcaggataacgacaaattcgtttggcgtccactagaggtatctgacagggtacaatctttacccccgacgttcagtctgtttgttaattctccagcatttaccccggctttcttgatggaattcttgaattcttatcatcagcaagcggattatttaagcacccattatgcagaacaaggaaatcacagattatttgaggcccaacgtaatttatttgcgggtgttagttttccagagtttaaagattcgccaagatggagacaaaccggtatcagcgtgttaaacactgaaatcaagaaacaagtatatgctgacggtatgcagtttgaactatctccaatttatcatgtcgcagcaattgacatattcttgaaggcttatggcagtgctaaacgtgtcaacttagaaaaggaatttccacagtcatacgtccagactgtagaaaacatgatcatggcccttatcagcatctcattgcctgattacaacactccaatgtttggtgatagttggataacggacaagaactttaggatggcacagttcgcaagctgggctagagtatttccagcaaatcaagctataaaatatttcgcgactgatggtaagcaagggaaggcccctaattttctatctaaagctttaagcaacgccggcttttatactttcaggtccggttgggacaagaatgcaactgtcatggtgttaaaagcgtctccacccggagaattccatgcgcaaccagacaatgggacattcgagttgtttattaaagggagaaattttaccccagatgctggagtgtttgtgtactcaggggatgaagcgataatgaagttaagaaattggtatcgtcaaaccagaatacattccacacttacactggacaaccaaaacatggtgattactaaggctagacaaaacaagtgggaaactggtaacaatctggacgttttgacatatacgaacccttcctatccaaatcttgaccatcagaggtcggtcttatttataaataagaaatacttcttggttatcgacagagccattggtgaagcaacaggtaatctgggtgttcattggcaattgaaagaggattctaatccagtgtttgacaagacgaagaacagggtatacacaacataccgtgatggtaataacttaatgatccagtccttaaatgctgatcgtacttctcttaatgaagaggaaggtaaggtttcctacgtgtacaataaagaactaaagagacccgcctttgtcttcgaaaagcccaagaagaatgccggaacgcagaatttcgtatctatcgtttacccttatgatggccagaaagctccagagataagcatccgtgagaacaaaggtaacgattttgagaaaggtaagcttaatttgacgctaacgattaatgggaaacagcaattagtcttagtaccataa

SEQ ID NO: 13 - DNA, 1968 bp, Pedobacter heparinus; identical to SEQ ID NO: 12
  reencoded nucleic acid sequence of glucuronosyl-disulfoglucosamine glucuronidase originating from Pedobacter heparinus with a N-terminal secretion signal and a C-terminal anchoring signal

SEQ ID NO: 14 - protein, 655 aa, Pedobacter heparinus
  amino acid sequence of glucuronosyl-disulfoglucosamine glucuronidase originating from Pedobacter heparinus with a N-terminal secretion signal
MQFSTVASVAFVALANFVAAQSSSITRKDFDHINLEYSGLEKVNKAVAAGNYDDAAKALLAYYREKSKAREPDFSNAEKPADIRQPIDKVTREMADKALVHQFQPHKGYGYFDYGKDINWQMWPVKDNEVRWQLHRVKWWQAMALVYHATGDEKYAREWVYQYSDWARKNPLGLSQDNDKFVWRPLEVSDRVQSLPPTFSLFVNSPAFTPAFLMEFLNSYHQQADYLSTHYAEQGNHRLFEAQRNLFAGVSFPEFKDSPRWRQTGISVLNTEIKKQVYADGMQFELSPIYHVAAIDIFLKAYGSAKRVNLEKEFPQSYVQTVENMIMALISISLPDYNTPMFGDSWITDKNFRMAQFASWARVFPANQAIKYFATDGKQGKAPNFLSKALSNAGFYTFRSGWDKNATVMVLKASPPGEFHAQPDNGTFELFIKGRNFTPDAGVFVYSGDEAIMKLRNWYRQTRIHSTLTLDNQNMVITKARQNKWETGNNLDVLTYTNPSYPNLDHQRSVLFINKKYFLVIDRAIGEATGNLGVHWQLKEDSNPVFDKTKNRVYTTYRDGNNLMIQSLNADRTSLNEEEGKVSYVYNKELKRPAFVFEKPKKNAGTQNFVSIVYPYDGQKAPEISIRENKGNDFEKGKLNLTLTINGKQQLVLVP

SEQ ID NO: 15 - protein, 725 aa, Pedobacter heparinus
  amino acid sequence of glucuronosyl-disulfoglucosamine glucuronidase originating from Pedobacter heparinus with a N-terminal secretion signal and a C-terminal anchoring signal
MQFSTVASVAFVALANFVAAQSSSITRKDFDHINLEYSGLEKVNKAVAAGNYDDAAKALLAYYREKSKAREPDFSNAEKPADIRQPIDKVTREMADKALVHQFQPHKGYGYFDYGKDINWQMWPVKDNEVRWQLHRVKWWQAMALVYHATGDEKYAREWVYQYSDWARKNPLGLSQDNDKFVWRPLEVSDRVQSLPPTFSLFVNSPAFTPAFLMEFLNSYHQQADYLSTHYAEQGNHRLFEAQRNLFAGVSFPEFKDSPRWRQTGISVLNTEIKKQVYADGMQFELSPIYHVAAIDIFLKAYGSAKRVNLEKEFPQSYVQTVENMIMALISISLPDYNTPMFGDSWITDKNFRMAQFASWARVFPANQAIKYFATDGKQGKAPNFLSKALSNAGFYTFRSGWDKNATVMVLKASPPGEFHAQPDNGTFELFIKGRNFTPDAGVFVYSGDEAIMKLRNWYRQTRIHSTLTLDNQNMVITKARQNKWETGNNLDVLTYTNPSYPNLDHQRSVLFINKKYFLVIDRAIGEATGNLGVHWQLKEDSNPVFDKTKNRVYTTYRDGNNLMIQSLNADRTSLNEEEGKVSYVYNKELKRPAFVFEKPKKNAGTQNFVSIVYPYDGQKAPEISIRENKGNDFEKGKLNLTLTINGKQQLVLVPGSAISQITDGQIQATTTATTEATTTAAPSSTVETVSPSSTETISQQTENGAAKAAVGMGAGALAAAAMLL

SEQ ID NO: 16 - DNA, 2154 bp, Saccharomyces cerevisiae
  nucleic acid sequence of glutamine-fructose-6-phosphate amidotransferase (GFA1) originating from Saccharomyces cerevisiae
atgtgtggtatctttggttactgcaattatctagtggaaagatccagaggagaaattatcgacaccttagtggatggtttacaaagattagaatatagaggctatgattccaccggtattgctatcgatggtgacgaagctgattctactttcatctataagcaaatcggtaaagtgagtgctttgaaagaggagattactaagcaaaatccgaacagagacgttacttttgtctctcattgtggtattgcgcatactagatgggctactcacggtcgaccagaacaagttaactgtcaccctcaaagatctgacccagaagaccaatttgtggtcgttcataatggtatcatcacaaattttagagaactgaagactcttttaattaacaaaggttataaattcgaaagtgataccgataccgagtgtattgctaaactatatttgcatttatacaatacaaatttacaaaatgggcatgacttagatttccacgaattaaccaagctagttcttttagaactagaaggttcatacgggttattatgtaaatcttgtcactatcctaatgaggttatcgccactagaaaagggtcccctttactgattggtgtcaaatctgaaaaaaaactaaaagtcgacttcgtggatgtggaatttcccgaagaaaacgctggtcaaccggaaattccattgaaatctaacaacaaatcatttggcttgggcccaaagaaagctcgtgaatttgaagctggttcccaaaatgccaatttactaccaattgccgccaatgaatttaacttgagacattctcaatccagggctttcctatcagaagatggatctccaacaccggtggaattttttgtttcttcggatgcggcatctgttgttaaacataccaagaaggtgctatttttagaagatgacgatttggctcatatttacgatggtgagttacatattcatagatctagaagagaagtaggcgcatcaatgacaaggtccattcaaactttagagatggagttagctcagatcatgaagggcccttacgaccattttatgcaaaaggaaatctatgagcaaccagaatctactttcaatactatgagaggtagaatcgactatgaaaataataaagtgatattgggtggtttaaaggcatggttaccagttgtcagaagagcacggagactgatcatgatcgcatgcggtacttcttatcattcatgtttggctactcgtgctatcttcgaagaattatcagatatcccagttagtgtggaattagcgtctgactttctggacagaaaatgccctgtcttcagagacgatgtatgcgtgtttgtttcacaaagtggtgaaactgcggataccatgctggctctaaattattgtttagaaagaggagccttaactgtcggaattgttaacagtgttggttcttctatctctcgtgtcacccactgtggtgttcatattaacgctggtcctgaaattggtgttgcctctacaaaagcttatacttcccagtatattgccttagtgatgtttgctctatcgctgtcagatgaccgtgtatcgaaaatagacagaagaattgaaatcattcaaggcttgaagttaatcccgggccaaattaagcaggtattaaagctggaaccaagaataaaaaagctctgtgcgactgaattaaaggatcaaaaatctctattgttattgggtagaggttaccaatttgctgctgctctggaaggtgctttgaagatcaaagaaatttcttatatgcattctgaaggtgttttggcaggtgagttgaagcacggtgtcttggccttggtggacgaaaacttgccaatcattgcttttggtaccagagactctctattccctaaagtagtttcctctattgagcaagttactgtaagaaagggccatccaattattatttgtaacgaaaatgatgaagtgtgggcgcaaaaatctaaatcaatcgacctgcaaaccttagaagttccacaaactgttgattgtttacaaggtctaattaatattattccattacaactaatgtcatattggttggctgttaataaagggattgatgttgattttccaagaaacttggctaaatctgttaccgtcgaataa

SEQ ID NO: 17 - DNA, 1788 bp, Chlorella virus
  reencoded nucleic acid sequence of glutamine-fructose-6-phosphate amidotransferase (GFA1) originating from Chlorella virus 1 (PBCV-1)
atgtgcgggatcttcggtgctgtgtcgaacaataatagcatagaagtttccatcaagggtatacagaagctagagtaccgcgggtacgattcgtgtggaatagcctatacagacggaggcgccattgaacggatcaggtcaatagatgggatcgacgacttaaggaagaaaacaataacagagtcttccccggtagctatcgctcattcaaggtggagtaccactgggattccaagtgttgtgaacgcccacccgcacatctctcggggcacgtctggatgcgagtcgcgcattgcagtcgtacacaatggcattattgaaaattaccagcagatccgcaagtacttgataaatcttgggtatactttcgattcacagaccgacacggaagtcattgctcatctaatagactcgcagtataacgggaacatcctgcacaccgtccaaatggcagttaagcacctcaaaggaagctacgccatagcagtcatgtgccataaggagtccgggaagatcgtggtggcaaaacagaagagtccgttggtattgggtataggaagcgacggagcttattatatcgcatcggacgtattggctcttcccactaacaaagtggtctacataagtgacggattctctgctgagttaagtcctggatcgatgactatctatgatctggatggcaataaggtagaatacgaggtggaagacgttgaaatggagcagacgtcgatgtctttagataactttgatcactacatgatcaaagagatcaatgaacagccgatatcaattctcaatactattaagaacaaaggattctatgcagagatatttggtgatctagcacatgaaatatttcagaagatcgacaatatattgattctagcctgcggaacttcgtatcatgcaggtctcgtcgggaagcaatggatagagaccatctcacgcatcccggtggatgttcatattgcaagcgagtatgagccgacaattccacgagccaatacacttgtgataacgattagtcaaagcggcgagactgcggatacaatcgcagcccttcaacgagcgcaaaacgcaggaatgatatacactttgtgtatctgcaacagccccaagtcaactctagtgcgcgagtcgataatgaaatacataaccaaatgcggttcagaagtttctgttgccagtactaaagcatttacgtcgcaacttgtggtgttgtatatgcttgcgaacgttcttgcgaacaaaactgatgatctgctaggggatcttcctcaagctatcgagagagtaatctgtcttactaatgacgaaatgaagcgttgggcggatgaaatttgtactgccaaaagtgccatcttcttagggagaggactgaacgcacctgtagcatttgagggcgcgctaaagttgaaggaaatctcatatatccatgccgagggtttcctcggtggagagttgaagcatgggccgctggctttgttggacgataagatccctgtaatcgtgactgttgctgaccacgcttatttggatcacattaaggctaacatcgacgaggtattggcacgaaacgttaccgtatacgcgatcgtcgatcagtatgttaatattgaaccacaggagcgactgcatgtagtaaaagtgccgtttgtgagtaaagagttttctcccatcatccacacaattccgatgcaattgttatcgtattatgtcgcgatcaagctgggcaagaacgttgacaagccacgtaacctggcgaaaagtgttacaacattctaa

SEQ ID NO: 18 - DNA, 1788 bp, Chlorella virus
  reencoded nucleic acid sequence of glutamine-fructose-6-phosphate amidotransferase (GFA1) originating from Chlorella virus 1 (PBCV-1)
atgtgtggcatctttggagcagtgtcaaacaacaactctatcgaggtgtcaatcaagggtattcagaagctagaatatcgtgggtatgattcgtgcggtattgcgtatacagatgggggtgcgattgagcgtatacgttctattgacggcattgacgatctgcgtaagaaaacaatcacagaatcatcaccagtggccattgctcactcgcggtggagcaccactggaattccatcagtggtgaacgcacatcctcatatttctcgcggaaccagtgggtgtgagtctcgtatcgcggtagtccacaacggtatcattgaaaactatcagcagatccgaaaatatctcatcaatcttggttatacgtttgatagtcaaacggacacagaggtcattgcgcatttgattgattctcagtacaatgggaatatcttgcacaccgtccaaatggctgtcaagcacctgaagggctcttatgccattgcagttatgtgtcataaagagtctggtaaaatagtcgtggcgaaacagaagtcacccctcgtacttggaatcggctcagatggtgcttactacatcgcttcggacgtgctggcgctgccgacaaataaagttgtttatatttcagacggtttctccgcagaactatctccagggagtatgaccatttacgatcttgatggaaataaagtagaatatgaagtagaggacgttgaaatggaacaaactagtatgtctctcgataactttgatcattacatgattaaggaaattaatgagcaaccaatcagtattctaaacactataaaaaataaagggttctatgcagaaatattcggtgatttggctcatgaaatcttccaaaaaatagacaacatcctgatactggcttgtggtacaagttatcacgccggtcttgtaggaaaacagtggatagagaccatctctagaatccccgtggatgttcacatcgcgagtgaatacgaacctactattccgagagcgaacacattggtaatcactatttcacagtcgggtgaaactgcggacacgatagcggctttgcaacgggcccaaaacgccgggatgatttatacattgtgtatttgcaattcaccaaagagcactcttgttcgtgagagcattatgaagtacatcacgaaatgtggttctgaggtgtcagtggcatcaacgaaggcgtttacttctcagctcgtagtactgtacatgctggcaaacgtattggcaaataaaaccgatgatttgctgggagacctcccacaggcaatagaacgggtaatttgtttgacaaatgacgaaatgaaacgatgggccgacgaaatttgcactgcgaaatccgcgatcttcctgggaagaggactaaacgcaccagttgcctttgagggagcgttgaagctcaaagaaatctcttacattcatgcagagggcttcctgggaggtgagttgaaacacggccccctcgcactccttgatgacaagattcctgttatcgtaaccgtagcagatcatgcttatttggaccatatcaaagcaaatatcgacgaagtgcttgcgaggaacgttacggtatacgccatagtagaccagtatgtgaacatcgagccccaggaacgccttcacgtcgtcaaggttccgtttgtatccaaagaattttctccgataattcacactatcccgatgcaactgctttcgtattacgtggcaattaagcttggaaagaacgttgacaaaccaaggaatcttgcaaaatccgtgactaccttttaa

SEQ ID NO: 19 - protein, 717 aa, Saccharomyces cerevisiae
  amino acid sequence of glutamine-fructose-6-phosphate amidotransferase (GFA1) originating from Saccharomyces cerevisiae
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 20 - protein, 595 aa, Chlorella virus
  amino acid sequence of glutamine-fructose-6-phosphate amidotransferase (GFA1) originating from Chlorella virus 1 (PBCV-1)
MCGIFGAVSNNNSIEVSIKGIQKLEYRGYDSCGIAYTDGGAIERIRSIDGIDDLRKKTITESSPVAIAHSRWSTTGIPSVVNAHPHISRGTSGCESRIAVVHNGIIENYQQIRKYLINLGYTFDSQTDTEVIAHLIDSQYNGNILHTVQMAVKHLKGSYAIAVMCHKESGKIVVAKQKSPLVLGIGSDGAYYIASDVLALPTNKVVYISDGFSAELSPGSMTIYDLDGNKVEYEVEDVEMEQTSMSLDNFDHYMIKEINEQPISILNTIKNKGFYAEIFGDLAHEIFQKIDNILILACGTSYHAGLVGKQWIETISRIPVDVHIASEYEPTIPRANTLVITISQSGETADTIAALQRAQNAGMIYTLCICNSPKSTLVRESIMKYITKCGSEVSVASTKAFTSQLVVLYMLANVLANKTDDLLGDLPQAIERVICLTNDEMKRWADEICTAKSAIFLGRGLNAPVAFEGALKLKEISYIHAEGFLGGELKHGPLALLDDKIPVIVTVADHAYLDHIKANIDEVLARNVTVYAIVDQYVNIEPQERLHVVKVPFVSKEFSPIIHTIPMQLLSYYVAIKLGKNVDKPRNLAKSVTTF

SEQ ID NO: 21 - DNA, 1434 bp, Saccharomyces cerevisiae
  nucleic acid sequence of UDP-N-acetylglucosamine pyrophosphorylase (QRI1) originating from Saccharomyces cerevisiae
atgactgacacaaaacagctattcattgaagccggacaaagtcaacttttccacaattgggaaagcttgtctcgcaaagaccaagaagaattgctttcaaacctggagcaaatatcttccaagaggtcccctgcaaaactactggaagactgtcaaaatgctattaaattctcactagctaactcttctaaggatactggcgtcgaaatttcaccattgccccctacttcgtacgagtcgcttattggcaacagtaagaaagaaaatgaatactggcgtttaggccttgaagctattggcaagggtgaagtcgcagtgattttaatggctggcggacaaggtacgcggttaggatcctctcaaccaaagggctgttacgacattggattgccttctaagaaatctctttttcaaattcaagctgaaaagttgatcaggttgcaagatatggtaaaggacaaaaaggtagaaattccttggtatattatgacatcaggccccactagagcggctactgaggcatactttcaagaacacaattattttggcttgaataaagaacaaattacgttcttcaaccagggaaccctgcctgcctttgatttaaccgggaagcatttcctaatgaaagacccagtaaacctatctcaatcaccagatggaaatggtggactctaccgtgccatcaaggaaaacaagttgaacgaagactttgataggagaggaatcaagcatgtttacatgtactgtgtcgataatgtcctatctaaaatcgcagaccctgtatttattggttttgccatcaagcatggcttcgaactggccaccaaagccgttagaaagagagatgcgcatgaatcagttgggttaattgctactaaaaacgagaaaccatgtgtcatagaatattctgaaatttccaatgaattggctgaagcaaaggataaagatggcttattaaaactacgcgcaggcaacattgtaaatcattattacctagtggatttactaaaacgtgatttggatcagtggtgtgagaatatgccatatcacattgcgaagaagaaaattccagcttatgatagtgttaccggcaagtacactaagcctaccgaaccaaacggtataaaattagagcaattcatatttgatgtctttgacactgtaccactgaacaagtttgggtgcttagaagtagatagatgcaaagaattttcacctttaaaaaacggtcctggttctaagaacgataatcctgagaccagcagactagcatatttgaaactaggaacctcgtggttggaagatgcaggcgctattgtaaaagatggggtactagtcgaagtttccagcaaattgagttatgcaggtgaaaatctatcccagttcaaaggtaaagtctttgacagaagtggtatagtattagaaaaataa

SEQ ID NO: 22 - protein, 477 aa, Saccharomyces cerevisiae
  amino acid sequence of UDP-N-acetylglucosamine pyrophosphorylase (QRI1) originating from Saccharomyces cerevisiae
MTDTKQLFIEAGQSQLFHNWESLSRKDQEELLSNLEQISSKRSPAKLLEDCQNAIKFSLANSSKDTGVEISPLPPTSYESLIGNSKKENEYWRLGLEAIGKGEVAVILMAGGQGTRLGSSQPKGCYDIGLPSKKSLFQIQAEKLIRLQDMVKDKKVEIPWYIMTSGPTRAATEAYFQEHNYFGLNKEQITFFNQGTLPAFDLTGKHFLMKDPVNLSQSPDGNGGLYRAIKENKLNEDFDRRGIKHVYMYCVDNVLSKIADPVFIGFAIKHGFELATKAVRKRDAHESVGLIATKNEKPCVIEYSEISNELAEAKDKDGLLKLRAGNIVNHYYLVDLLKRDLDQWCENMPYHIAKKKIPAYDSVTGKYTKPTEPNGIKLEQFIFDVFDTVPLNKFGCLEVDRCKEFSPLKNGPGSKNDNPETSRLAYLKLGTSWLEDAGAIVKDGVLVEVSSKLSYAGENLSQFKGKVFDRSGIVLEK

SEQ ID NO: 23 - DNA, 1713 bp, Saccharomyces cerevisiae
  nucleic acid sequence of Phosphoglucomutase-1 (PGM1) originating from Saccharomyces cerevisiae
atgtcacttctaatagattctgtaccaacagttgcttataaggaccaaaaaccgggtacttcaggtttacgtaagaagaccaaggttttcatggatgagcctcattatactgagaacttcattcaagcaacaatgcaatctatccctaatggctcagagggaaccactttagttgttggaggagatggtcgtttctacaacgatgttatcatgaacaagattgccgcagtaggtgctgcaaacggtgtcagaaagttagtcattggtcaaggcggtttactttcaacaccagctgcttctcatataattagaacatacgaggaaaagtgtaccggtggtggtatcatattaactgcctcacacaacccaggcggtccagagaatgatttaggtatcaagtataatttacctaatggtgggccagctccagagagtgtcactaacgctatctgggaagcgtctaaaaaattaactcactataaaattataaagaacttccccaagttgaatttgaacaagcttggtaaaaaccaaaaatatggcccattgttagtggacataattgatcctgccaaagcatacgttcaatttctgaaggaaatttttgattttgacttaattaaaagcttcttagcgaaacagcgcaaagacaaagggtggaagttgttgtttgactccttaaatggtattacaggaccatatggtaaggctatatttgttgatgaatttggtttaccggcagaggaagttcttcaaaattggcaccctttacctgatttcggcggtttacatcccgatccgaatctaacctatgcacgaactcttgttgacagggttgaccgcgaaaaaattgcctttggagcagcctccgatggtgatggtgataggaatatgatttacggttatggccctgctttcgtttcgccaggtgattctgttgccattattgccgaatatgcacccgaaattccatacttcgccaaacaaggtatttatggcttggcacgttcatttcctacatcctcagccattgatcgtgttgcagcaaaaaagggattaagatgttacgaagttccaaccggctggaaattcttctgtgccttatttgatgctaaaaagctatcaatctgtggtgaagaatccttcggtacaggttccaatcatatcagagaaaaggacggtctatgggccattattgcttggttaaatatcttggctatctaccataggcgtaaccctgaaaaggaagcttcgatcaaaactattcaggacgaattttggaacgagtatggccgtactttcttcacaagatacgattacgaacatatcgaatgcgagcaggccgaaaaagttgtagctcttttgagtgaatttgtatcaaggccaaacgtttgtggctcccacttcccagctgatgagtctttaaccgttatcgattgtggtgatttttcgtatagagatctagatggctccatctctgaaaatcaaggccttttcgtaaagttttcgaatgggactaaatttgttttgaggttatccggcacaggcagttctggtgcaacaataagattatacgtagaaaagtatactgataaaaaggagaactatggccaaacagctgacgtcttcttgaaacccgtcatcaactccattgtaaaattcttaagatttaaagaaattttaggaacagacgaaccaacagtccgcacatag

SEQ ID NO: 24 - protein, 570 aa, Saccharomyces cerevisiae
  amino acid sequence Phosphoglucomutase-1 (PGM1) originating from Saccharomyces cerevisiae
MSLLIDSVPTVAYKDQKPGTSGLRKKTKVFMDEPHYTENFIQATMQSIPNGSEGTTLVVGGDGRFYNDVIMNKIAAVGAANGVRKLVIGQGGLLSTPAASHIIRTYEEKCTGGGIILTASHNPGGPENDLGIKYNLPNGGPAPESVTNAIWEASKKLTHYKIIKNFPKLNLNKLGKNQKYGPLLVDIIDPAKAYVQFLKEIFDFDLIKSFLAKQRKDKGWKLLFDSLNGITGPYGKAIFVDEFGLPAEEVLQNWHPLPDFGGLHPDPNLTYARTLVDRVDREKIAFGAASDGDGDRNMIYGYGPAFVSPGDSVAIIAEYAPEIPYFAKQGIYGLARSFPTSSAIDRVAAKKGLRCYEVPTGWKFFCALFDAKKLSICGEESFGTGSNHIREKDGLWAIIAWLNILAIYHRRNPEKEASIKTIQDEFWNEYGRTFFTRYDYEHIECEQAEKVVALLSEFVSRPNVCGSHFPADESLTVIDCGDFSYRDLDGSISENQGLFVKFSNGTKFVLRLSGTGSSGATIRLYVEKYTDKKENYGQTADVFLKPVINSIVKFLRFKEILGTDEPTVRT

SEQ ID NO: 25 - DNA, 1500 bp, Saccharomyces cerevisiae
  nucleic acid sequence of UTP-glucose-1-phosphate uridylyltransferase (UGP1) originating from Saccharomyces cerevisiae
atgtccactaagaagcacaccaaaacacattccacttatgcattcgagagcaacacaaacagcgttgctgcctcacaaatgagaaacgccttaaacaagttggcggactctagtaaacttgacgatgctgctcgcgctaagtttgagaacgaactggattcgtttttcacgcttttcaggagatatttggtagagaagtcttctagaaccaccttggaatgggacaagatcaagtctcccaacccggatgaagtggttaagtatgaaattatttctcagcagcccgagaatgtctcaaacctttccaaattggctgttttgaagttgaacggtgggctgggtacctccatgggctgcgttggccctaaatctgttattgaagtgagagagggaaacacctttttggatttgtctgttcgtcaaattgaatacttgaacagacagtacgatagcgacgtgccattgttattgatgaattctttcaacactgacaaggatacggaacacttgattaagaagtattccgctaacagaatcagaatcagatctttcaatcaatccaggttcccaagagtctacaaggattctttattgcctgtccccaccgaatacgattctccactggatgcttggtatccaccaggtcacggtgatttgtttgaatctttacacgtatctggtgaactggatgccttaattgcccaaggaagagaaatattatttgtttctaacggtgacaacttgggtgctaccgtcgacttaaaaattttaaaccacatgatcgagactggtgccgaatatataatggaattgactgataagaccagagccgatgttaaaggtggtactttgatttcttacgatggtcaagtccgtttattggaagtcgcccaagttccaaaagaacacattgacgaattcaaaaatatcagaaagtttaccaacttcaacacgaataacttatggatcaatctgaaagcagtaaagaggttgatcgaatcgagcaatttggagatggaaatcattccaaaccaaaaaactataacaagagacggtcatgaaattaatgtcttacaattagaaaccgcttgtggtgctgctatcaggcattttgatggtgctcacggtgttgtcgttccaagatcaagattcttgcctgtcaagacctgttccgatttgttgctggttaaatcagatctattccgtctggaacacggttctttgaagttagacccatcccgttttggtccaaacccattaatcaagttgggctcgcatttcaaaaaggtttctggttttaacgcaagaatccctcacatcccaaaaatcgtcgagctagatcatttgaccatcactggtaacgtctttttaggtaaagatgtcactttgaggggtactgtcatcatcgtttgctccgacggtcataaaatcgatattccaaacggctccatattggaaaatgttgtcgttactggtaatttgcaaatcttggaacattga

SEQ ID NO: 26 - protein, 499 aa, Saccharomyces cerevisiae
  amino acid sequence of UTP--glucose-1-phosphate uridylyltransferase (UGP1) originating from Saccharomyces cerevisiae
MSTKKHTKTHSTYAFESNTNSVAASQMRNALNKLADSSKLDDAARAKFENELDSFFTLFRRYLVEKSSRTTLEWDKIKSPNPDEVVKYEIISQQPENVSNLSKLAVLKLNGGLGTSMGCVGPKSVIEVREGNTFLDLSVRQIEYLNRQYDSDVPLLLMNSFNTDKDTEHLIKKYSANRIRIRSFNQSRFPRVYKDSLLPVPTEYDSPLDAWYPPGHGDLFESLHVSGELDALIAQGREILFVSNGDNLGATVDLKILNHMIETGAEYIMELTDKTRADVKGGTLISYDGQVRLLEVAQVPKEHIDEFKNIRKFTNFNTNNLWINLKAVKRLIESSNLEMEIIPNQKTITRDGHEINVLQLETACGAAIRHFDGAHGVVVPRSRFLPVKTCSDLLLVKSDLFRLEHGSLKLDPSRFGPNPLIKLGSHFKKVSGFNARIPHIPKIVELDHLTITGNVFLGKDVTLRGTVIIVCSDGHKIDIPNGSILENVVVTGNLQILEH

SEQ ID NO: 27 - DNA, 480 bp, Saccharomyces cerevisiae
  nucleic acid sequence of Glucosamine 6-phosphate N-acetyltransferase (GNA1) originating from Saccharomyces cerevisiae
atgagcttacccgatggattttatataaggcgaatggaagagggggatttggaacaggtcactgagacgctaaaggttttgaccaccgtgggcactattacccccgaatccttcagcaaactcataaaatactggaatgaagccacagtatggaatgataacgaagataaaaaaataatgcaatataaccccatggtgattgtggacaagcgcaccgagacggttgccgctacggggaatatcatcatcgaaagaaagatcattcatgaactggggctatgtggccacatcgaggacattgcagtaaactccaagtatcagggccaaggtttgggcaagctcttgattgatcaattggtaactatcggctttgactacggttgttataagattattttagattgcgatgagaaaaatgtcaaattctatgaaaaatgtgggtttagcaacgcaggcgtggaaatgcaaattagaaaatag

SEQ ID NO: 28 - protein, 159 aa, Saccharomyces cerevisiae
  amino acid sequence of Glucosamine 6-phosphate N-acetyltransferase (GNA1) originating from Saccharomyces cerevisiae
MSLPDGFYIRRMEEGDLEQVTETLKVLTTVGTITPESFSKLIKYWNEATVWNDNEDKKIMQYNPMVIVDKRTETVAATGNIIIERKIIHELGLCGHIEDIAVNSKYQGQGLGKLLIDQLVTIGFDYGCYKIILDCDEKNVKFYEKCGFSNAGVEMQIRK

SEQ ID NO: 29 - DNA, 1674 bp, Saccharomyces cerevisiae
  nucleic acid sequence of phosphoacetylglucosamine mutase (PCM1) originating from Saccharomyces cerevisiae
atgaaggttgattacgagcaattgtgcaaactctacgatgacacgtgccgcacaaagaatgtgcagttcagttacggtacggccggattcagaacgctggccaagaatttggatacggtgatgttcagtactggtatactggcggttctcaggtcgctgaagcttcagggtcagtatgtgggggtgatgatcacggcgtcgcacaacccataccaggacaacggggtcaagatcgtggaaccagacggatcgatgcttttggccacatgggagccatatgccatgcagttggccaatgcggcctcttttgccactaattttgaagaatttcgtgttgagttggccaagctgattgaacacgaaaagattgatttgaatacaaccgtcgtgcctcacatcgtggttgggagagactctagggaaagtagtccatacttgctgcgctgcttgacttcctccatggccagcgtcttccacgcgcaagttttggacctaggctgtgtcactacgcctcaattgcattacattactgatttgtccaacaggcggaaactggaaggagacacagcgccagttgccacagaacaggactactattcgttctttataggagccttcaacgagctcttcgccacgtatcagctggagaagaggctgtctgtcccaaaattgttcatagacacagccaatggtatcggtggtccacagttgaaaaaactactggcctccgaagattgggacgtgccagcggagcaagttgaggtaatcaacgacaggtccgatgttccagaactgttgaattttgaatgcggtgcggattatgtgaagactaaccagagattacccaagggtctttctccatcctcgtttgattcgctatattgctcctttgatggtgacgcagacagggttgtgttctactatgtcgactcaggatcaaaatttcatttgttggatggtgacaaaatttccactttgtttgcaaagttcttgtctaaacaactagaattggcacacctagaacattctttgaagattggtgttgtgcaaactgcctatgcaaacggcagttccaccgcttacataaaaaatacgttgcactgtcccgtgtcttgcactaagacaggtgttaaacacttgcatcatgaagctgccactcagtacgatattggcatttatttcgaagcaaatggacatggtacgattatattcagcgaaaaatttcatcgaactatcaaatctgaattatccaagtccaagttaaatggtgatacgttagctttgagaactttgaagtgtttctctgaattgattaatcagaccgtgggagatgctatttcagacatgcttgctgtccttgctactttggcgattttgaaaatgtcgccaatggattgggatgaagagtatactgatttgcccaacaagctggttaagtgcatcgttcctgataggtcaattttccaaaccacggaccaggaaagaaaattgctcaatccagtggggttgcaagacaagatagatcttgtggtagccaagtatcccatgggaagaagctttgtcagagccagtggtacggaggatgcggtgagggtttatgcggaatgtaaggactcctctaagttaggtcaattttgtgacgaagtggtggagcacgttaaggcatctgcttga

SEQ ID NO: 30 - protein, 557 aa, Saccharomyces cerevisiae
  amino acid sequence of phosphoacetylglucosamine mutase (PCM1) originating from Saccharomyces cerevisiae
MKVDYEQLCKLYDDTCRTKNVQFSYGTAGFRTLAKNLDTVMFSTGILAVLRSLKLQGQYVGVMITASHNPYQDNGVKIVEPDGSMLLATWEPYAMQLANAASFATNFEEFRVELAKLIEHEKIDLNTTVVPHIVVGRDSRESSPYLLRCLTSSMASVFHAQVLDLGCVTTPQLHYITDLSNRRKLEGDTAPVATEQDYYSFFIGAFNELFATYQLEKRLSVPKLFIDTANGIGGPQLKKLLASEDWDVPAEQVEVINDRSDVPELLNFECGADYVKTNQRLPKGLSPSSFDSLYCSFDGDADRVVFYYVDSGSKFHLLDGDKISTLFAKFLSKQLELAHLEHSLKIGVVQTAYANGSSTAYIKNTLHCPVSCTKTGVKHLHHEAATQYDIGIYFEANGHGTIIFSEKFHRTIKSELSKSKLNGDTLALRTLKCFSELINQTVGDAISDMLAVLATLAILKMSPMDWDEEYTDLPNKLVKCIVPDRSIFQTTDQERKLLNPVGLQDKIDLVVAKYPMGRSFVRASGTEDAVRVYAECKDSSKLGQFCDEVVEHVKASA

SEQ ID NO: 31 - DNA, 574 bp, Artificial
  Synthetic nucleic acid sequence of promotor pTDH3
ctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 32 - DNA, 585 bp, Artificial
  Synthetic nucleic acid sequence of promotor pTDH3.Sk
caattcatcggcccttttagcggctacccgcgccatctaaatgatagggcgggtgacactatggtaaatcccataattaggtgtctgggtgagtggttctgatgccggcatccactaaatatattggagcccattttttacgcgggcttccagaaaaaaagagaatcccagcaccaaaaggtggttctcttcaccaaccatcagatcataggtccacaaccacacataacaggggcacaaaaaggcaaaaaacggacataacctcaatggagtgatgcaaattgactggagcaaaagctgacacaaggcattgattgacctacgcatgtatctgtattcttttcttacaccttctattaccttctaactctttgggttggaaaaaactgaaaaaaaaggttgggacctggttcccccaagttgtccccctacttggttattaaatatataaagacagcaagtgttgattataatcttgtaaatctatagttcttaatctatacttctatttatattttaaattagtctttttatttccaagtccccaagaacttagtttcgaataaacacacacaaataaacaca

SEQ ID NO: 33 - DNA, 700 bp, Artificial
  Synthetic nucleic acid sequence of promotor pTDH3-1.sba
gcagcgcttcttccgctctagtttttatagttattattactaccaccttaaaaatacgtaaatactcaaaatagtagtgatattcccaaccttattcatccaaggcacatcatcatcatcagccattcatctttcacctgccattagtaacccgtcttctcattgagcgggttacggcagccacaggccacattccgaatgtctgggtgagcggtcccttttccagcatccactaaatatctccgatcccgctttttaatctggcttcctgaaaaaaagagaatcccagcaccaaaaaatggctctcttcaccaaccatcagatcataggtcccattctcttaccgcaaccgtacagaacaggggaaaacgggtacaacctcaatggagtgatgcaaactgactggagcaaaaagctgacacaaggcaatcgacctacgtgtctgtctattttctcacaccttctattaccttctaactctctgggttggaaaaaactgaaaaaaaggttgagaccagtttccacaaatcatccccctgtttgattaataaatatataaagacgacaactatcgatcataaactcataaaactataactcctttacacttcttattttatagttattctattttaattcttattgattttaaaaccccaagaacttagtttcgaaaacacacacacacaaacaattaaaa

SEQ ID NO: 34 - DNA, 602 bp, Artificial
  Synthetic nucleic acid sequence of promotor pTDH3.Sar
gagctaaatatcagcccttcgggtcctgcctgctacccggtcctgttcgaataaaaacgcgggtaacacgacccagtaacacctgtcgttgggtgtctgggtcagaagttctgataccggcttccactaaatagattgggttccgctctttacgctggcttcctgaaaaaagagattccgggcaccaaaaaattggtctctttgccaaccatcagatcataggtccattctcttaccataaccacacaggataggggcaccacaggcgaaaatgggcacaaaatctcaatggagtgatgcaaattagctggaacaaaagctgacacaaggcaattaacctgcgcatgtatccatctccttttcttacaccttctcttacgttctaactgtttgggttggaaaaattaaaaaaaaaaggttgagaccagtttccccaaatcgtccccctacttgattcataaatatataaagacgacaactattgattataatcttgtaaatctataactctttactttctcctatttataatttaacttaatctttttagatttaaaaccccaagaacttagtttcgaacaaacacacacaaataaacaaaa

SEQ ID NO: 35 - DNA, 550 bp, Artificial
  Synthetic nucleic acid sequence of promotor pENO2
cgctcagcatctgcttcttcccaaagatgaacgcggcgttatgtcactaacgacgtgcaccaacttgcggaaagtggaatcccgttccaaaactggcatccactaattgatacatctacacaccgcacgccttttttctgaagcccactttcgtggactttgccatatgcaaaattcatgaagtgtgataccaagtcagcatacacctcactagggtagtttctttggttgtattgatcatttggttcatcgtggttcattaattttttttctccattgctttctggctttgatcttactatcatttggatttttgtcgaaggttgtagaattgtatgtgacaagtggcaccaagcatatataaaaaaaaaaagcattatcttcctaccagagttgattgttaaaaacgtatttatagcaaacgcaattgtaattaattcttattttgtatcttttcttcccttgtctcaatcttttatttttattttatttttcttttcttagtttctttcataacaccaagcaactaatactataacatacaataata

SEQ ID NO: 36 - DNA, 598 bp, Artificial
  Synthetic nucleic acid sequence of promotor pTEF3
ggctgataatagcgtataaacaatgcatactttgtacgttcaaaatacaatgcagtagatatatttatgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 37 - DNA, 383 bp, Artificial
  Synthetic nucleic acid sequence of promotor pTEF1
gtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctacgatacagttctcacatcacatccgaacataaacaacc

SEQ ID NO: 38 - DNA, 383 bp, Artificial; identical to SEQ ID NO: 37
  Synthetic nucleic acid sequence of promotor pTEF1.ago

SEQ ID NO: 39 - DNA, 498 bp, Artificial
  Synthetic nucleic acid sequence of promotor pTEF1.Sba
cgccaacaaaccttcgaacactttaattttcgaggaccgcagatcctcacatcacacccacacccaagactgcttcccccacacaccctgcatctgtacactttcttctgctctgtttttctctccggcgttctctcgggtcgcccgcatcgccgcgccggctggaaccgcccacgcaccgcatattgcaaatcgcctgccccctcttgctcctttttgagggcgcgccgttacccgcgcccagggtccggaaaaagaaacaaggctctaccgcgtttctttttccttgtcgaaaaaggcaaaaatgaaaatttttatcacgtttctttttttttgaaaaattttttttttggttttttttctttcgatggcctcccattgatatttaagttaataaatggttttcagttttcaagtttcagtttgtgttcttctttgctaactttcacttacacctcgaaagaaagtatagcaatctaatcttagttttaattacaa

SEQ ID NO: 40 - DNA, 700 bp, Artificial
  Synthetic nucleic acid sequence of promotor pPDC1
ttatttacctatctctaaacttcaacaccttatatcataactaatatttcttgagataagcacactgcacccataccttccttaaaaacgtagcttccagtttttggtggttccggcttccttcccgattccgcccgctaaacgcatatttttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatcaatcaaa

SEQ ID NO: 41 - DNA, 998 bp, Artificial
  Synthetic nucleic acid sequence of promotor pCCW12
aaccagggcaaagcaaaataaaagaaacttaatacgttatgccgtaatgaagggctaccaaaaacgataatctcaactgtaaacaggtacaatgcggacccttttgccacaaaacatacatcattcattgccggaaaaagaaagaagtgaagacagcagtgcagccagccatgttgcgccaatctaattatagatgctggtgccctgaggatgtatctggagccagccatggcatcatgcgctaccgccggatgtaaaatccgacacgcaaaagaaaaccttcgaggttgcgcacttcgcccacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaataaaagccaatcctcgaagcgtgggtggtaggccctggattatcccgtacaagtatttctcaggagtaaaaaaaccgtttgttttggaattccccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaata

SEQ ID NO: 42 - DNA, 976 bp, Artificial
  Synthetic nucleic acid sequence of promotor pCCW12.Sm
cgttgtgctgtagtgaaggaagactaaaaaggataatcacagttgtaaagaggtataatgcggacccttttgccacaaaacacacatctttcgtttccaaaataagaaagaagaaagcaaaaagattagcagccacgttgctgcgatctaattatagacggtggcgtcatcattctcacccaagattgtgtcttgaacctgccacgggtcctgcgttatcgccggatgtaaaactagacatgcaaaaaaaggaccttccaggtagcgtgctccacaccacccatgaccaccacagttagtccaaaagaggcagcaccacttcccgatgggggaattagattgctgccaccctcacctcactaatgctgcggtgtgcggatatgccctgctatatatagctccgcgtttttgaaccagcacagcgcgagcaccaaaaaggaaaatcgcataggcccagaactgatttcagcacgggctattggcgttgggtttccgttctgggaaaccttcgccgcgtccccctcacaaacctccgcacaagttcgagcaagcgggaaaaaacgaaaaacgccattaatactaaataaagcaaatcctcgaagcgtgggtggcaagcccctggatttttccgcacaagtactcttctcaggagtaaaaaaacccgtttgttttggaattccccatttcgcggccatctacgccggtatcttcgcaatatctatcagcgataactcagcaattttaatattcgtgttgcagtgctgcgatagcgggagtcttgtttgtaacataacggcagaaaggaatgagagaaaattttccattctttggcctccgttcaagtatataaagccggcatgcttggtaatctttctctctcttctgtattgtttctataatacttttatcttctaattattttctgaaaaaaccaagaaattaatcttctgtcattcgcttaaacactatatcaata

SEQ ID NO: 43 - DNA, 750 bp, Artificial
  Synthetic nucleic acid sequence of promotor pCCW12.Sk
tgtaaaatcctacacgcaaaaaacctcttgggttgcgcgttttccaccacccacgacccgcacaatcaatccaaaaggggcaacgccagttcccaatgcgggaattagctcctcacccccctcacccgctaacgctgcggtgtgcggacacgcagcagtatttatagatcctcgtgttggaaccagcccgcgtgagcaccaaattggaaagtcgcaatgggcccagaaccgctttcagtactgggccattggcgtctagtttccgttttgagcgtccttcgccgcgtctctctgtgaaatccccgcacaagtctgagcaggcaaaaaaaaaaaacgccaccaaaaataaataaagccaatcctcgaaggatgagtaggaaaggaagcccctggttttttcccgcacgaatatttttcaggagtgaaaaaatccgtttgttttggaattccccatttcgcgctcacctacgccggtatctttggaacaactatcagcgataactcagcaaaatttgcatatccgtgctgcaatagtgcgatagtgggattgggagtcttgttgcatcataacggcagaaaggaatgaataaaaattttccgttctttgtctccgttcaagtatataaagtcggcatgcttgattatcttttcttctcttctactacatttctatttcactttctactctattcttccctgaaaaacccaagaaaataatcttctgtcattcgcttaaacaatatatcaaaa

SEQ ID NO: 44 - DNA, 750 bp, Artificial
  Synthetic nucleic acid sequence of promotor pCCW12.Sba
accatgccacggtgctggccccacttccacccacgacccgcacggttggcccgaaaggggcaacaccagttcccaatacggaaattagtccgccaccaccctcacctcgctagagctgcggtgtgcgggcgtgcatcgctatttatagacatgcctgctggcgtcaccgcgcgcgagcaccaaacaggaaaatcgcactgggcccagaaccacgctatgcgctgggccgatggcgtccggtttccctttgggagccccctgccacgttcgcctaacaaatccccgcaccggcttgagaaaaaagcgaaaagcgaaaaaaaaaaatcaacgccaccaaaattaaaaaaaaagagccatcctcgaagggtgaatagtagcccctgacttttcccgcacagacagacacctttcaggagtgaacaaaaaagcagtttgttttggaattcccccatttcgcggtggcctgcgcaggtatctctgcgtcaactatcagcgataactcagcaaattttgcatattcgtgttgcgatactacgataatgggagtctgtcgcctaataacggcaacaaggaatgagagagaaaaattttcttcattctccagctcccgttcaagtatataaggtcggcatggtcgattgtctttccttctcttcagttacgtctctctatttacattattcttatttttatttaataaaaaccccaagaaattaatcttctgtcattcgcttaaacactatatcaaaa

SEQ ID NO: 45 - DNA, 750 bp, Artificial
  Synthetic nucleic acid sequence of promotor pCCW12.Sar
aaaaaacaaccttctgccaacctgcgtgcttctcaccacccatgacccacacaattgacccgaatggggcaactccagttcccaatacgggaattaactcgccaccatatttaccgcgttgaagctgtggtgtgcggacactccgtactatttatagacccacgcggtggaaccagcacgcgcgcgcactaaacaggaaaatcgcattgagtccagaaccgccaccagcacttggccattggcgtctaatttccgttttcggcgcccctcaccgcgtccttctaacaaagcgcgcacaagcttgagcaagtgaaaagaaaattaaaaataaaaaaccgccaccaaaacaaataaagcaatcttcgaagtgtgggttggtgggaagcccctggcttttcccgcaccagtcgttttcaggagtaaaaaaatacccgtttgttttggaattccccatttcgcggcgacctgcgccggtatctttgcaacaactatttgcgataactcagcaaaatttgcatattcgtgttgggatattgcgatagtgggagtcttgttgcataataacggtaaaaagaagtgaaggaaaaaaatttgcatcctttagtctcagttcaagtatataaagtcgggatattcaattatctttctttctcttgctcaaaggtttctatattttttttatagtatttcttttgttataaaataccaagaaattaatcttctgtcattcgcttaaatactacatcaata

SEQ ID NO: 46 - DNA, 500 bp, Artificial
  Synthetic nucleic acid sequence of promotor pNUP57
tcatctgcgcaatgactatcaagaccttctgcaagaatttcaaatctcactgaaaatcttgaccgaaaagtgtcttgaaaacccatcaagcctgcaaaacctatctttgacattagtctccattataaaaacggcatagttgggagaaaactttccatacttcaattgtggactgatataagtattttagttttgcccgcatgatcatcccacatggctacagcagttctctcataggaaaatagtacaatagctacgtgatataatctaaataattgttgccaatgtgtaattatatcattttgaacgtacgcgaaatggattattttcaaaaattttgtttcttgaaatgagtaaaagcaaaagtccaactctccaagtcgatgtaaacaactttttgccaaagggactgaaagactaaatcgaggattatcccgttcaaactattccagaaacgctcgttagtaacaaaagacataccttgttgaccaattgatcac

SEQ ID NO: 47 - DNA, 523 bp, Artificial
  Synthetic nucleic acid sequence of promotor pCCW10.ago
ggtaccacggcaacctcgttcgctgttcatccccttcgtcacacaggacgttggatgccgtaagcagcgttgcttttgatcctcaggatcggccgggtaacccgcggctgcttctattttagtattcatatctcaagcacatccattccggccgtttgggggcgccgccgcactcgtgtccattcctaccgtggcacttagggctatcctgtcggagcgccccgccgaccgccttatcggcaccaaaagtagaagccccggccccgcgtggctcagactcaccatcggtgctatttacttttcgatcagatcgcggcgcgcggtggccggcatttccggaagcggccacggagcagaggtggcgcattcgaatcgcatacgtcttcgccacgccggaaaaaaaattttcggctatataaggagaggcggccgtcttgctgcaggcagtttcactttctctaaaaccaaagaacatcgatttctttagtcactcgcttccttacaccgaactcgaggcggccgc

SEQ ID NO: 48 - DNA, 943 bp, Artificial
  Synthetic nucleic acid sequence of promotor pCWP2
ctagcctcccctttttattttgtgcggtcaccgcaagggacaaagcttttcttagaaaaccgtctgagaagcataacgtacgccatcccctagacatattaataatgctacagatactatgctgctcgtctttttttgacgacccttttattgcaatgtgcaactaatggcaaacaaccacatagtatcacagtattacattgcctccaccgatgcggatgttagggcgccaagtctgtcatgaagcatgttcctgtcataatcttgtatgcaaaataccgcgttctgcgccactgatatgctaggcagcagcaacctatgcagaagattgcttttcccacgcctgttttacgtctccagggcacttgaaacaatgcagcgatcgccgccacaacacgccaaagagaagcgaaagtgggcctgggcggcctcagtttcggcagaggtaaacaacacgaactgaactgccttagctccgaagggcaattccacaggcactccgcggggcccggccaaggcccaaaaggcgtggaatatgcgcgttttggggccataacacccagtaccacggccggaacgggccatataataagtttttcactctcaagaatggtaaacgtaaataggaacatcccactaccctagaaattgcggaaatttcgcgcttatcattagaaaatctggaaccgtcctttttcctctttcttgcatttccctttccgtattattgccattctttaactgcatttggggaaccgtagaccaaaagccaaacagagaaatgtaacgttctaaaaaaaaaacaacgaaaaaattgaaaaataagatacaataatcgtatataaatcaggcttcttgttcatcattttcaattctcttcttgccatcccttttcctatctttgttcttttcttctcataatcaagaataaataacttcatca

SEQ ID NO: 49 - DNA, 750 bp, Artificial
  Synthetic nucleic acid sequence of promotor pCCW120.Sca
tattttttttaaattaaagatgctgtgcaattaattgtccggagggacccttgcatactggccagtttgtacgggcgttacatagcgccgatcgacgaacagcttattaatagcaagatttgtttctcccatgagcaattaaccagtaattttcacagaaactagcacaaaaccaaccaaacagttaaaggcagtttcggagaataaacaacgcgctcactgagcttgctgttgccaaaaataacagtcgcgtgcgatggcgggcttggcattttcctcgaaggggaaacgccgccagggttcgctgcggtgtgtgggtggggcagcccccgggcgcccagacggtgccgcagggccttgagacagcccaaaatgccgcgaaaacgcaagggcgcggtgacctctttcctaatgcggaaagccccaccgccagcgacgtgacgcgttgtgcgggctcttctcgggacccggaaaaagaaatacgtacacaaacaccagcaaattttttcgcttgagaggctgcccgtgtttctgcgcaacgtgaaacacggtggacaagacaagagagattgacaagcgtcgttatcgtcccagtgaggtttcccaattatataaatacatccctgttggtggattcttaattacaacttccatctctctctctctcacttcttttgttcccttacatatccccaagaaatcaatcttctgccattcgctttaacaacaaacaaacttttaacttttaaa

SEQ ID NO: 50 - DNA, 535 bp, Artificial
  Synthetic nucleic acid sequence of promotor pRPLA1
tcaagttggatactgatctgatctctccgccctactaccagggaccctcatgattaccgctcgaatgcgacgtttcctgcctcataaaactggcttgaaaatatttattcgctgaacagtagcctagcttataaaaatttcatttaattaatgtaatatgaaaactcacatgccttctgtttctaaaattgtcacagcaagaaataacattaccatacgtgatcttattaaactctagtatcttgtctaatacttcatttaaaagaagccttaaccctgtagcctcatctatgtctgctacatatcgtgaggtacgaatatcgtaagatgataccacgcaactttgtaatgattttttttttttcattttttaaagaatgcctttacatggtatttgaaaaaaatatctttataaagtttgcgatctcttctgttctgaataatttttagtaaaagaaatcaaaagaataaagaaatagtccgctttgtccaatacaacagcttaaaccgattatctctaaaataacaagaagaa

SEQ ID NO: 51 - DNA, 505 bp, Artificial
  Synthetic nucleic acid sequence of promotor pCUP1
cggcaaacttcaacgatttctatgatgcattttataattagtaagccgatcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcacataaa

SEQ ID NO: 52 - DNA, 510 bp, Artificial
  Synthetic nucleic acid sequence of promotor pMET6
ccacaggaaatatttcacgtgacttacaaacagagtcgtacgtcaggaccggagtcaggtgaaaaaatgtgggccggtaaagggaaaaaaccagaaacgggactactatcgaactcgtttagtcgcgaacgtgcaaaaggccaatatttttcgctagagtcatcgcagtcatggcagctctttcgctctatctcccggtcgcaaaactgtggtagtcatagctcgttctgctcaattgagaactgtgaatgtgaatatggaacaaatgcgatagatgcactaatttaagggaagctagctagttttcccaactgcgaaagaaaaaaaggaaagaaaaaaaaattctatataagtgatagatatttccatctttactagcattagtttctcttttacgtattcaatatttttgttaaactcttcctttatcataaaaaagcaagcatctaagagcattgacaacactctaagaaacaaaataccaatataatttcaaagtacatatcaaaa

SEQ ID NO: 53 - DNA, 686 bp, Artificial
  Synthetic nucleic acid sequence of promotor pMET25
ttacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcattattttttgctttttctcttgaggtcacatgatcgcaaaatggcaaatggcacgtgaagctgtcgatattggggaactgtggtggttggcaaatgactaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataca

SEQ ID NO: 54 - DNA, 363 bp, Artificial
  Synthetic nucleic acid sequence of promotor pSAM1
gaaacggacgtaagacggaaatagaatttgaagataaagttatatatcactacacacgaatactttcttttttttttttcacaggaaaactgtggtggcgcccttgcctactagtgcatttcttttttcgggttcttgtctcgacgaaattttagcctcatcgtagtttttcactctggtatcgatgaaaaagggaagagtaaaaagttttccgtttagtacttaatgggattggtttgggacgtatatatcgactggtgttgtctgttattcatcgttgtttttcggttagcttcgaaaaaaaaatagagtaaaaaccaggaatttaccctaaaaacaagaaaaaataagataaacgaaaat

SEQ ID NO: 55 - DNA, 300 bp, Artificial
  Synthetic nucleic acid sequence of terminator tTPI1
gattaatataattatataaaaatattatcttcttttctttatatctagtgttatgtaaaataaattgatgactacggaaagcttttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattactttgtctctaaatccttgtaaaatgtgtacgatctctatatgggttactca

SEQ ID NO: 56 - DNA, 299 bp, Artificial
  Synthetic nucleic acid sequence of terminator tMET25
gtgtgcgtaatgagttgtaaaattatgtataaacctactttctctcacaagtactatacttttataaaacgaactttattgaaatgaatatcctttttttcccttgttacatgtcgtgactcgtactttgaacctaaattgttctaacatcaaagaacagtgttaattcgcagtcgagaagaaaaatatggtgaacaagactcatctacttcatgagactactttacgcctcctataaagctgtcacactggataaatttattgtaggaccaagttacaaaagaggatgatggaggttt

SEQ ID NO: 57 - DNA, 300 bp, Artificial
  Synthetic nucleic acid sequence of terminator tDIT1
taaagtaagagcgctacattggtctacctttttgttcttttacttaaacattagttagttcgttttctttttctcatttttttatgtttcccccccaaagttctgattttataatattttatttcacacaattccatttaacagagggggaatagattctttagcttagaaaattagtgatcaatatatatttgcctttcttttcatcttttcagtgatattaatggtttcgagacactgcaatggccctagttgtctaagaggatagatgttactgtcaaagatgatattttgaatttc

SEQ ID NO: 58 - DNA, 300 bp, Artificial
  Synthetic nucleic acid sequence of terminator tRPL3
gaagttttgttagaaaataaatcattttttaattgagcattcttattcctattttatttaaatagttttatgtattgttagctacatacaacagtttaaatcaaattttctttttcccaagtccaaaatggaggtttattttgatgacccgcatgcgattatgttttgaaagtataagactacatacatgtacatatatttaaacatgtaaacccgtccattatattgcttactttcttcttttttgccgttttgacttggacctctggtttgctatttccttacaatctttgctacaat

SEQ ID NO: 59 - DNA, 304 bp, Artificial
  Synthetic nucleic acid sequence of terminator tRPL3.sm
gaagtttttaaagcattttttagacacttctcatttttctaagtttttttaaaatagttttatgtatttactacgtatcacaatttgaaataattcatcttcccaaaaaactaagatttttatccttgtcacgatccgtaaccagtttataatattttagagcttatacacgtacgtatacacacgtgtcggtacatgagaattacgttcaaaattattcactttttttttctctgccgttttacttttgaactctgtctcgctatttccttacaatcttcgctacaataccacttgcccttgg

SEQ ID NO: 60 - DNA, 299 bp, Artificial
  Synthetic nucleic acid sequence of terminator tRPL3.sba
gaagttttctgacaaaaacataacgttttttccaatcatttcttatttttccggtttatttaaatagtttttatgtactattatacgtatgactatttaacttaaattcttcctcccaagaaatctcccaagtttttcattatcatggcatacaccactatcagttacaaaatggtagctcaaccatatatatatctctatatacacatataaatgcaaacaggtccaagtcaccgctcactgcagtttcttttgccgttttgacttcgatctctgcttggctattttctcacaatcct

SEQ ID NO: 61 - DNA, 300 bp, Artificial
  Synthetic nucleic acid sequence of terminator tRPL41B
gcggattgagagcaaatcgttaagttcaggtcaagtaaaaattgatttcgaaaactaatttctcttatacaatcctttgattggaccgtcatcctttcgaatataagattttgttaagaatattttagacagagatctactttatatttaatatctagatattacataatttcctctctaataaaatatcattaataaaataaaaatgaagcgatttgattttgtgttgtcaacttagtttgccgctatgcctcttgggtaatgctattattgaatcgaagggctttattatattaccct

SEQ ID NO: 62 - DNA, 300 bp, Artificial
  Synthetic nucleic acid sequence of terminator tRPL15A
gctggttgatggaaaatataattttattgggcaaacttttgtttatctgatgtgttttatactattatctttttaattaatgattctatatacaaacctgtatattttttctttaaccaatttttttttttatagacctagagctgtacttttattctgctatcaagcaaacccctaccccctcttctcaatcctcccctcaggcagaacttatctacctgtatcaaggagcggacgagggagtcctaattgttctacgtataccaatgctagcagcttacataggtggtggcactacca

SEQ ID NO: 63 - DNA, 300 bp, Artificial
  Synthetic nucleic acid sequence of terminator tRPL15A.sba
gctggctgatgaaaaatatagttctgttgggcaagcatttgtttacctagcatctcttttatactattattatctttatatttgatgattttatatacaagttgtataccttttctttaaccaatttttttttttctaatggtgcacctagaagtacattttttctcaccaatagatagtcaagatactcccagcctctatggcgttaccacggagccgacaagggaaagctgcttatcttacatatgcagatgccaaggcccgttacaggccgccttattgatgtttagaaatagcttc

SEQ ID NO: 64 - DNA, 300 bp, Artificial
  Synthetic nucleic acid sequence of terminator tIDP1
tcgaatttacgtagcccaatctaccacttttttttttcattttttaaagtgttatacttagttatgctctaggataatgaactacttttttttttttttttttactgttatcataaatatatataccttattgttgtttgcaaccgtcggttaattccttatcaaggttccccaagttcggatcattaccatcaatttccaacattttcatgagttcttcttcttcattaccgtgttttagggggctgttcgcacttctaatagggctatcaccaagctgttctaattcgtccaaaagtt

SEQ ID NO: 65 - DNA, 300 bp, Artificial
  Synthetic nucleic acid sequence of terminator tTEF1.sba
ggagattgataggacttttctagttgcatatcttttatttttaaatcttatctattagttaattttttgtaatttatccttatatatatagtttggttattctaaaacatcatttcagtatctaaaacctctcttattcattacctttttatttaatggtttttgctacaggcaaaaatttaatggtttttgctacaggcaaaaatcccgccgtggacttattccacgttaactcggttacagggtcatgaaccattttgtcaattatcgaaataacttcttcaaaagtccctcttactt

SEQ ID NO: 66 - DNA, 60 bp, Artificial
  Synthetic nucleic acid sequence of the secretion sequence
atgcaatttagcacagtcgcatcagtagccttcgttgccttggccaacttcgtggcagca

SEQ ID NO: 67 - protein, 20 aa, Artificial
  Synthetic amino acid sequence of the secretion sequence added in N-ter
MQFSTVASVAFVALANFVAA

SEQ ID NO: 68 - DNA, 213 bp, Artificial
  Synthetic nucleic acid sequence of the anchoring sequence
ggatccgccatttctcaaatcactgacggtcaaatccaagctactaccactgctaccaccgaagctaccaccactgctgccccatcttccaccgttgaaactgtttctccatccagcaccgaaactatctctcaacaaactgaaaatggtgctgctaaggccgctgtcggtatgggtgccggtgctctagctgctgctgctatgttgttataa

SEQ ID NO: 69 - protein, 68 aa, Artificial
  Synthetic amino acid sequence of the anchoring sequence added in C-ter
AISQITDGQIQATTTATTEATTTAAPSSTVETVSPSSTETISQQTENGAAKAAVGMGAGALAAAAMLL